(19) United States
(12) Patent Application Publication
(10) Pub. No.: US 2017/0218091 A1
(43) Pub. Date: Aug. 3, 2017

(54) MONOVALENT BINDING PROTEINS

(71) Applicant: AbbVie Inc., North Chicago, IL (US)

(72) Inventor: Dominic AMBROSI, Worcester, MA (US)

(21) Appl. No.: 15/323,472

(22) PCT Filed: Jul. 2, 2015
(86) PCT No.: PCT/US2015/039110

§ 371 Date: Jan. 3, 2017

Related U.S. Application Data
(60) Provisional application No. 62/020,804, filed on Jul. 3, 2014.

(51) Int. Cl. C07K 16/46; C07K 16/24; G01N 33/68; A61K 39/395; A61K 45/06

(52) U.S. Cl. CPC C07K 16/468; A61K 39/395; A61K 45/06; G01N 33/6869; C07K 16/244; C07K 16/241; G01N 33/6863; C07K 2317/51; C07K 2317/515; C07K 2317/31; C07K 2317/526; C07K 2317/53; C07K 2317/52; C07K 2317/35; C07K 2317/94; C07K 2317/76; G01N 2333/54; G01N 2333/525

(57) ABSTRACT

Disclosed herein are engineered monovalent binding proteins that bind to one or more antigens, as well as methods of making and using the binding proteins in the prevention, diagnosis, and/or treatment of disease.

### RELATED APPLICATIONS

The present application claims the benefit of priority to U.S. Provisional Patent Application No. 62/020,504, filed Jul. 3, 2014, the contents of which are hereby incorporated by reference herein in their entirety.

### FIELD OF THE DISCLOSURE

The present disclosure relates to novel monovalent binding protein formats having improved stability.

### BACKGROUND

Target-binding proteins that possess preferable pharmacodynamic and pharmacokinetic features have attracted increasing attention in the effort to develop biological therapeutic agents. Substantial work has been dedicated to the optimization of immunoglobulin amino acid sequences in order to obtain constructs having superior therapeutic effects. These modified immunoglobulins may have different structures and properties from those found in naturally existing immunoglobulins and may provide superior therapeutic effects.

An immunoglobulin is a useful platform for drug development because of its various desirable intrinsic properties. For example, immunoglobulins typically have high target specificity, superior biostability and bioavailability, less toxicity, and sufficient target binding affinity to maximize therapeutic effects. However, neutralizing certain targets such as cell-surface receptors with regular or bivalent immunoglobulins has been challenging due to unexpected triggering of certain signal transduction pathways. For example, a large number of CD40 antibodies stimulate, rather than B cell proliferation (Adams et al. (2005) J. Immunol. 174:542-550; Malmborg Hager et al. (2003) Scand. J. Immunol. 517:517-523). Targeting CD28 on the surface of T cells with antibody JJ316 and 5.11 was reported to elicit a super-agonistic effect, presumably by crosslinking neighboring CD28 homodimers to form a large scale lattice structure (Hunig et al. (2005) Immunol. Letters 100:21-28).

Monovalent antibodies do not typically exhibit the “cross-linking” effect seen for multivalent antibodies. Nevertheless, monovalent antibodies have not been regarded as desirable therapeutics because certain inherent features in their structure/architecture may limit their application. For example, a monovalent antibody in Fab form can exhibit inferior pharmacodynamics (e.g., it is unstable in vivo and rapidly cleared following administration). Furthermore, as compared with their multivalent counterparts, monovalent immunoglobulins generally have lower apparent binding affinity due to the absence of avidity binding effects.

Full length immunoglobulins have been the immunoglobulin of choice for many immunotherapeutics, which is likely due to their biostability in vivo. Nevertheless, monovalent immunoglobulins may be acceptable where biostability is not as critical a factor for therapeutic efficacy, as compared to other factors such as bioavailability that might be improved by a monovalent format. For example, due in part to superior tissue penetration as compared to full length antibodies, monovalent Fabs may be better vehicles for delivery of heterologous molecules such as toxins to target cells or tissues. See e.g., U.S. Pat. No. 5,169,939, incorporated herein by reference. Other examples where monovalent antibodies are being developed as therapeutics include settings where monovalency is critical for obtaining a therapeutic effect. For instance, monovalency may be preferred when bivalency of an antibody may induce a target cell to undergo antigenic modulation. Examples of monovalent antibodies are described in Cobbold and Waldmann (1984) Nature 308:460-462; EP Patent No. EP0131424; Glennie and Stevenson (1982) Nature 295:712-714; Nielsen and Routledge (2002) Blood 100:4067-4073; Stevenson et al. (1989) Anticancer Drug Des. 3(4):219-230; Routledge et al (1995) Transplant. 605347-853; Clark et al. (1989) l Eur. J. Immunol. 19:381-388; Bolt et al. (1993) Eur. J. Immunol. 23:403-411; Routledge et al. (1991) Eur. J. Immunol. 21:2717-2725; Staerz et al. (1985) Nature 314:628-631; and U.S. Pat. No. 5,968,509.

Notably, a monovalent antibody fragment may contain functional dimeric Fc sequences, which are included because their effector functions (e.g., complement-mediated lysis of T cells) are needed for therapeutic function. Further, antibodies that contain fully functional Fc regions have longer half-lives necessary for therapeutic activity. Due to the practical difficulties of obtaining such antibodies, while avoiding multivalent contaminants, there has been much reluctance to include an Fc region in monovalent antibodies where the Fc region is not necessary for therapeutic function. Existing antibody production technology does not provide an efficient method for obtaining large quantities of sufficiently purified heterodimers comprising a single antigen binding component (i.e., monovalency) and an Fc region.

Several approaches have been tested to increase the in vivo stability of immunoglobulin fragments, yet none has met with complete success. For example, a Fab fragment may be attached to stability moieties such as polyethylene glycol or other stabilizing molecules such as heterologous peptides. See e.g., Dennis et al. (2002) J. Biol. Chem. 277:35035-35043; PCT Publication No. WO/01145746, each incorporated herein by reference. An anti c-Met monovalent molecule MetMAb with a Fab-Fc/Fc structure is in clinical trials for non-small cell lung cancer. See PCT Publication No. WO2005063816, incorporated herein by reference. An Fc fragment has been connected to the C-terminus of a light chain, then coupled with a full heavy chain to achieve monovalent binding to antigen. See PCT Publication No. WO20070105199, incorporated herein by reference. Monovalent binding may also be achieved by replacing an IgG1 backbone with an IgG4 backbone. See PCT Publication No. WO2007059782, incorporated herein by reference. However, these showed weak CH3-mediated dimerization and exhibited other unfavorable properties.

In view of the above, there remains a significant need for improved monovalent binding protein formats (including antibody, dual variable domain, and other multispecific formats), as well as methods of producing and using monovalent binding proteins, for example as therapeutic agents. Identifying ways to construct monovalent constructs, including monovalent dual variable domain immunoglobulins, may lead to improvements in preventing, diagnosing, and/or treating disorders. Also, while a variety of structures have been provided in the art, with various advantages and disadvantages, the disclosed monovalent, multi- or mono-specific therapeutic binding proteins may offer further improvements over the existing constructs.

### SUMMARY

Disclosed herein are monovalent binding proteins capable of binding one or more antigens. The disclosed binding proteins are particularly advantageous in that they are highly stable and can be produced in an efficient manner utilizing standard transfection and purification procedures. In certain embodiments, the disclosure provides an “Ambromab” format for a monovalent, multi- or mono-specific therapeutic binding protein that utilizes knobs-into-holes mutations to combine two different heavy chain Fc regions. In the Ambromab format, a first heavy chain comprises a heavy chain Fc, a hinge region (e.g., all or part of an IgG hinge), and at least one heavy chain variable region, whilst a second chain comprises a heavy chain Fc, a modified hinge region, a human Cκ (hCκ) or Cλ (hCλ) light chain constant region and at least one light chain variable region. Ambromab molecules can be monovalent multi-specific or monovalent monospecific.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, VH2 is a second heavy chain variable domain, L1 is a linker, CH1, CH2 and CH3 are heavy chain constant domains 1, 2 and 3, respectively, X1 comprises a first immunoglobulin hinge region; and wherein the second polypeptide chain comprises VL1-L2-VL2-CL-X2-CH2-CH3,wherein VL1 is a first light chain variable region, L2 is a linker, VL2 is a second light chain variable region, CL is a light chain constant domain, CH2 and CH3 are heavy chain constant domains 2 and 3, respectively; and X2 comprises a second immunoglobulin hinge region; wherein the first and second polypeptide chains comprise a hetero-dimerization motif that facilitates the monovalent dimerization of the first and second polypeptide chains, wherein VH1 and VL1 form one functional binding site for antigen A (i.e., any first antigen target), and VH2 and VL2 form one functional binding site for antigen B (i.e., any second antigen target, which is chosen independently of the antigen A target). The constant domains can comprise wild-type sequences or can comprise variants modified to retain essential effector functions while also promoting formation of a monovalent construct. In one embodiment, X2 on the second polypeptide chain is a modified immunoglobulin hinge region.

In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains. Suitable leucine zipper domains are described in Kostelny et al. (1992) J. Immunol. 148:1547-1553, which is incorporated by reference in its entirety. In an embodiment, the hetero-dimerization motif is located in the CH3 domain of the first and second polypeptide chains.

In an embodiment, the modified second immunoglobulin hinge region comprises an amino acid deletion, insertion or substitution. In an embodiment, the modified second immunoglobulin hinge region comprises an altered cysteine residue. In an embodiment, the altered cysteine residue enhances the hetero-dimerization of the first and second polypeptide chains as compared to the hetero-dimerization of first and second polypeptide chains comprising an unmodified second immunoglobulin hinge region. In an embodiment, the altered cysteine is the N-terminal cysteine of the second immunoglobulin hinge region.

In an embodiment, at least one of the first or second immunoglobulin hinge regions is modified. In an embodiment, at least one of the first or second immunoglobulin hinge regions comprises at least 4 continuous amino acids from the amino acid sequence EPKSCDKTHTCPPC. In an embodiment, the first immunoglobulin hinge region comprises the amino acid sequence EPKSCDKTHT. In an embodiment, the modified second immunoglobulin hinge region comprises the amino acid sequence EPKSXDKTHT, wherein X denotes an altered cysteine. In an embodiment, the modified second immunoglobulin hinge region comprises the amino acid sequence EPKSXDKTHT, wherein X is any amino acid except cysteine. In an embodiment, the modified second immunoglobulin hinge region comprises the amino acid sequence EPKSXDKTHT, wherein X is alanine. In an embodiment, the DKTHT sequence of EPKSXDKTHT in X1 is replaced in the first immunoglobulin hinge region with the amino acid sequence VE. In an embodiment, the EPKSXDKTHT amino acid sequence within the modified second immunoglobulin hinge region is replaced with the amino acid sequence VE. In an embodiment, the EPKSXDKTHT amino acid sequence within the modified second immunoglobulin hinge region is replaced with the amino acid sequence VE. In an embodiment, the EPKSX sequence of EPKSXDKTHT in the modified second immunoglobulin hinge region is deleted. In an embodiment, the EPKSX sequence of EPKSXDKTHT in the modified second immunoglobulin hinge region is deleted.

In an embodiment, the first and second immunoglobulin hinge regions are IgG1 hinge regions or modified versions thereof that retain at least one function of a wild-type hinge region. In an embodiment, the light chain constant domain is a C_{κ} kappa constant domain. In an embodiment, the first and second polypeptide chains are covalently linked. In an embodiment, antigens A and B are the same antigen.

In various embodiments, a binding protein comprising a first and second polypeptide chain is disclosed, wherein the first polypeptide chain comprises VH1-CH1-X1-CH2-CH3, wherein VH1 is a first heavy chain variable domain, CH1, CH2 and CH3 are heavy chain constant domains 1, 2 and 3, respectively, and X1 comprises a first immunoglobulin hinge region; and wherein the second polypeptide chain comprises VL1-CL-X2-CH2-CH3, wherein VL1 is a first light chain variable region, CL is a light chain constant domain, CH2 and CH3 are heavy chain constant domains 2 and 3; and X2 comprises a second immunoglobulin hinge region, wherein the first and second polypeptide chains comprise a hetero-dimerization motif that facilitates the dimerization of the first and second polypeptide chains, and wherein VH1 and VL1 form a functional binding site for an antigen. The constant domains can comprise wild-type sequences or can comprise variants modified to retain essential effector functions while also promoting formation of a monovalent construct. In one embodiment, X2 on the second polypeptide chain is a modified immunoglobulin hinge region.

In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In an embodiment of the second aspect, the second immunoglobulin hinge region is modified to comprise an amino acid deletion, insertion or substitution. In an embodiment of the second aspect, the modified second immunoglobulin hinge region comprises an altered cysteine residue. In an embodiment, the altered cysteine residue enhances the hetero-dimerization of the first and second polypeptide chains as compared to the hetero-dimerization of first and second polypeptide chains comprising an unmodified second immunoglobulin hinge region. In an embodiment, the altered cysteine is the N-terminal cysteine of the second immunoglobulin hinge region. In an embodiment, at least one of the first or the second immunoglobulin hinge regions comprises at least 4 continuous amino acids from the amino acid sequence EPKSCDKTHTCPPC. In an embodiment, the first immunoglobulin hinge region comprises the amino acid sequence EPKSCDKTHT. In an embodiment, the modified second immunoglobulin hinge region comprises the amino acid sequence EPKSXDKTHT, wherein X denotes an altered cysteine. In an embodiment, the modified second immunoglobulin hinge region comprises the amino acid sequence EPKSXDKTHT, wherein X is any amino acid except cysteine. In an embodiment, the modified second immunoglobulin hinge region comprises the amino acid sequence EPKSXDKTHT, wherein X is alanine. In an embodiment, the DKTHT sequence of EPKSXDKTHT within the first immunoglobulin hinge region is replaced with the amino acid sequence VE. In an embodiment, the EPKSXDKTHT amino acid sequence within the modified second immunoglobulin hinge region is replaced with the amino acid sequence VE. In an embodiment, the EPKSXDKTHT amino acid sequence within the modified second immunoglobulin hinge region is replaced with the amino acid sequence VE. In an embodiment, the EPKSX sequence of EPKSXDKTHT within the modified second immunoglobulin hinge region is deleted. In an embodiment, the EPKSX sequence of EPKSXDKTHT within the modified second immunoglobulin hinge region is deleted.

In an embodiment, the first and second immunoglobulin hinge regions are IgG1 hinge regions or modified versions thereof that retain at least one function of a wild-type hinge region. In an embodiment of the second aspect, the light chain constant domain is a C_{κ} kappa constant domain. In an embodiment, the first and second polypeptide chains are covalently linked.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first IgG1 hinge region comprising the amino acid sequence EPKSCDKTHT, and wherein the second polypeptide chain comprises VL1-L2-VL2-CK-X2-CH2-CH3, wherein VL1 is a first light chain variable region, L2 is a linker. VL2 is a second light chain variable region. CK is a kappa light chain constant domain, X2 comprises a modified second IgG1 hinge region comprising the amino acid sequence EPKSXDKTHT, wherein X denotes a substitution of a cysteine residue with alanine, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In a various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first IgG1 hinge region comprising the amino acid sequence EPKSCDKTHT, and wherein the second polypeptide chain comprises VL1-L2-VL2-C_{κ}-X2-CH2-CH3, wherein VL1 is a first light chain variable region, L2 is a linker, VL2 is a second light chain variable region, C_{κ} is a kappa light chain constant domain, X2 comprises a modified second IgG1 hinge region, wherein EPKSC of the EPKSCDKTHT amino acid sequence is deleted, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first IgG1 hinge region comprising the amino acid sequence EPKSCDKTHT, and wherein the second polypeptide chain comprises VL1-L2-VL2-CK-X2-CH2-CH3, wherein VL1 is a first light chain variable region, L2 is a linker, VL2 is a second light chain variable region, C_{κ} is a kappa light chain constant domain, X2 comprises a second modified IgG1 hinge region wherein the EPKSXDKTHT amino acid sequence is replaced with the amino acid sequence VE, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first hinge region, wherein the DKTHT sequence of the EPKSXDKTHT amino acid sequence is replaced with the amino acid sequence VE and wherein the second polypeptide chain comprises VL1-L2-VL2-CK-X2-CH2-CH3, wherein VL1 is a first light chain variable region, L2 is a linker, VL2 is a second light chain variable region, C_{κ} is a kappa light chain constant domain, X2 comprises a modified second IgG1 hinge region comprising the amino acid sequence EPKSXDKTHT, wherein X denotes a substitution of a cysteine residue with alanine, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first IgG1 hinge region, wherein the DKTHT sequence of the EPKSXDKTHT amino acid sequence is replaced with the amino acid sequence VE and wherein the second polypeptide chain comprises VL1-L2-VL2-CK-X2-CH2-CH3, wherein VL1 is a first light chain variable region, L2 is a linker, VL2 is a second light chain variable region, C_{κ} is a kappa light chain constant domain, X2 comprises a modified second IgG1 hinge region, wherein EPKSC of the EPKSCDKTHT amino acid sequence is deleted, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In various embodiments, a binding protein comprising a first and second polypeptide chain is disclosed, wherein the first polypeptide chain comprises VH1-L1-VH2-CH1-X1-CH2-CH3, wherein VH1 is a first heavy chain variable domain, L1 is a linker, VH2 is a second heavy chain variable domain, CH1, CH2 and CH3 are heavy chain constant domains 1, 2 and 3, respectively, X1 comprises a first IgG1 hinge region, wherein the DKTHT sequence of EPKSXDKTHT in the X1 is replaced with the amino acid sequence VE and wherein the second polypeptide chain comprises VL1-L2-VL2-CK-X2-CH2-CH3, wherein VL1 is a first light chain variable region, L2 is a linker. VL2 is a second light chain variable region, C_{κ} is a kappa light chain constant domain, X2 comprises a second modified IgG1 hinge region wherein the EPKSXDKTHT amino acid sequence in X2 is replaced with the amino acid sequence VE, CH2 and CH3 are heavy chain constant domains 2 and 3, respectively; and wherein the first and second polypeptide chains comprise a hetero-dimerization motif that facilitates the dimerization of the first and second polypeptide chains, and wherein VH1 and VL1 form one functional binding site for antigen A, and VH2 and VL2 form one functional binding site for antigen B. In an embodiment, the hetero-dimerization motif is located in the CH3 domain of the first and second polypeptide chains. In an embodiment, the hetero-dimerization motif comprises knobs-into-holes mutations in the CH3 domains of the first and second polypeptide chains. In an embodiment, the hetero-dimerization motif comprises leucine zipper domains linked to the first and second polypeptide chains.

In an embodiment, the binding protein binds to a cytokine selected from TNFα, VEGF, or PDGF (although the binding protein can bind other antigens, including other cytokines in addition to the listed cytokines, in various embodiments). In an embodiment, the binding protein binds to a receptor, including, but not limited to ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C-Met; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mep-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLPSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDR1); CXCL13; CXCL14; CXCL16; (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DLL4, DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A.; FGF; FGF1 (aFGF); FGF10; FGF11; FGF12; FGF12B; FGF13; FGF14; FGF16; FGF17; FGF18; FGF19; FGF2 (bFGF); FGF 20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HLAC4; HDAC5; HDAC7A; HDAC9; HGF; HIF1A; HIP1; histamine and histamine receptors; HLA-A; HLA-DRA; HM^{74;} HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1R1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-1); LTB; LTB4R (GPR16); LTB4R2; LTBR; MACMARCKS; MAG or Omgp; MAP2K7 (c-Jun); MDK; MIB1; midkine; MIF; MIP-2; MK167 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1.; PF4 (CXCL4); PGE, PGE2, PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; TB4R2; T3X21; TCP10; TDGF1; TEK; TGFA; TGFB1; TGFB111; TGFB2; TGFB3; TGFB1; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; or ZFPM.

In various embodiments, the L1 and L2 linkers are independently either present or absent. In some embodiments, at least one linker between variable domains in a binding protein comprises AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 51; RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22). GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); GGGGSGGGGS (SEQ ID NO: 29); GGSGGGGSG (SEQ ID NO: 30); or G/S based sequences (e.g., G4S and (G4S repeats; SEQ ID NO: 31). In an embodiment, the linker is a cleavable linker. In an embodiment, the linker is cleavable by one or more enzyme or agent selected from the group consisting of a zinc-dependent endopeptidase, Matrix Metalloproteinase (MMP), a serralysin, an astacin, an adamalysin, MMP-1; MMP-2; MMP-3; MMP-7; MMP-8; MMP-9; MMP-10; MMP-11; MMP-12; MMP-13; MMP-14; MMP-15; MMP-16; MMP-17; MMP-18; MMP-19; MMP-20; MMP-21; MMP-22; MMP-23A; MMP-23B; MMP-24; MMP-25; MMP-26; MMP-27; MMP-28; a Disintegrin and Metalloproteinase (ADAM); ADAM17; ADAMTS1; ADAM1; ADAM10; ADAM8; ADAMTS4; ADAMTS13; ADAM12; ADAM15; ADAM9; ADAMTS5; ADAM33; ADAM11; ADAM2; ADAMTS2; ADAMTS9; ADAMTS3; ADAMTS7; ADAM22; ADAM28; ADAMTS12; ADAM19; ADAMTS8; ADAM29; ADAM23; ADAM3A; ADAM18; ADAMTS6; ADAM7; ADAMDES1; ADAM20; ADAM6; ADAM21; ADAM3B; ADAMTSL3; ADAMTSL4; ADAM30; ADAMTS20; ADAMTSL2; a Caspase; Caspases 1-12, Caspase 14; a Cathepsin; Cathepsin G; Cathepsin B; Cathepsin D; Cathepsin L1; Cathepsin C; Cathepsin K; Cathepsin S; Cathepsin H; Cathepsin A; Cathepsin E; Cathepsin L; Cathepsin Z; Cathepsin F; Cathepsin G-like 2; Cathepsin L-like 1; Cathepsin W; Cathepsin L-like 2; Cathepsin L-like 3; Cathepsin L-like 4; Cathepsin L-like 5; Cathepsin L-like 6; Cathepsin L-like 7; Cathepsin O; a Calpain; Calpain 3; Calpain 10; Calpain 1 (mu/l) large subunit; Calpain; small subunit 1; Calpain 2, (mu/l); large subunit; Calpain 9; Calpain 11; Calpain 5; Calpain 6; Calpain 13; Calpain 8; Calpain, small subunit 2; Calpain 15; Calpain 12; Calpain 7; and Calpain 8.

In an embodiment, the binding protein has an on rate constant (Kₒₙ) to one or more target of at least about 10² M⁻¹s⁻¹; at least about 10³ M⁻¹s⁻¹; at least about 10⁴ M⁻¹s⁻¹; at least about 10⁵ M⁻¹s⁻¹; or at least about 10⁵ M⁻¹s⁻¹, as measured by surface plasmon resonance. In an embodiment, the binding protein has an on rate constant (Kₒₙ) to one or more target from about 10² M⁻¹s⁻¹ to about 10³ M⁻¹s⁻¹; from about 10³ M⁻¹s⁻¹ to about 10⁴ M⁻¹s⁻¹; from about 10⁴ M⁻¹s⁻¹ to about 10⁵ M⁻¹s⁻¹; or from about 10⁵ M⁻¹s⁻¹ to about 10⁶ M⁻¹s⁻¹, as measured by surface plasmon resonance.

In an embodiment, the binding protein has an off rate constant (K_{off}) for one or more target of at most about 10³ s⁻¹; at most about 10⁻⁴s⁻¹; at most about 10⁻⁵ s⁻¹; or at most about 10⁻⁶ s⁻¹, as measured by surface plasmon resonance. In an embodiment, the binding protein has an off rate constant (K_{off}) to one or more target of about 10⁻³ s⁻¹ to about 10⁻⁴ s⁻¹; of about 10⁻⁴ s⁻¹ to about 10⁻⁵ s⁻¹, or of about 10⁻⁵ s⁻¹ to about 10⁻⁶ s⁻¹, as measured by surface plasmon resonance.

In an embodiment, the binding protein has a dissociation constant (K_{d}) to one or more target of at most about 10⁻⁷M; at most about 10⁻⁸ M; at most about 10⁻⁹ M; at most about 10⁻¹⁰M; at most about 10⁻¹¹M; at most about 10⁻¹²M; or at most 10⁻¹³ M. In an embodiment, the binding protein has a dissociation constant (K_{d}) to one or more target of about 10⁻¹⁰ M to about 10⁻¹⁰ M; of about 10⁻⁸ M to about 10⁻⁹ M; of about 10⁻⁹ M to about 10⁻¹⁰ M; of about 10⁻¹⁰ M to about 10⁻¹¹ M; of about 10⁻¹¹ M to about 10⁻¹² M; or of about 10⁻¹² to about 10⁻¹³ M,

In an embodiment, the binding protein is a conjugate further comprising an agent. In an embodiment, the agent is an immunoadhesion molecule, an imaging agent, a therapeutic agent, or a cytotoxic agent. In an embodiment, the imaging agent is a radiolabel, an enzyme, a fluorescent label, a luminescent label, a bioluminescent label, a magnetic label, or biotin. In an embodiment, the radiolabel is ³H, ¹⁴C, ³⁵S, ⁹⁰Y, ⁹⁹TC, ¹¹¹In, ¹²⁵I, ¹³¹I, ¹⁷⁷Lu, ¹⁶⁶Ho, or ¹⁵³Sm. In yet an embodiment, the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent.

In an embodiment, the binding protein is a crystallized binding protein and exists as a crystal. In an embodiment, the crystal is a carrier-free pharmaceutical controlled release crystal. In an embodiment, the crystallized binding protein has a greater half-life in vivo than the soluble counterpart of the binding protein. In an embodiment, the crystallized binding protein retains biological activity.

In an embodiment, the binding protein described herein is glycosylated. For example, the glycosylation pattern is a human glycosylation pattern.

In an embodiment, the binding protein comprises any of the paired VH and VL sequences shown tables 1 and 2 that together form an antigen binding site. The paired VH and VL can be in either the inner or outer domain. In an embodiment, the binding protein comprises the set of CDR sequences from an antigen binding domain shown in tables 1 and 2 (i.e., CDRs 1-3 from a VH sequence in table 1, and CDRs 1-3 from the paired VL sequence in table 2, with the CDRs arranged in the order shown in the tables). In an embodiment, the CDR regions are those identified by the Kabat numbering scheme.

An isolated nucleic acid or group of nucleic acids encoding any one of the binding proteins disclosed herein is also provided. A further embodiment provides a vector comprising the isolated nucleic acid(s) disclosed herein wherein the vector is pcDNA; pTT; pTT3 (pTT with additional multiple cloning site); pEFBOS; pBV; pJV; pcDNA3.1 TOPO; pEF6 TOPO; pBOS; pHybE; or pBJ.

In another aspect, a host cell is transformed with one or more of the vectors disclosed herein. In an embodiment, the host cell is a prokaryotic cell, for example, E. coli. In an embodiment, the host cell is a eukaryotic cell, for example, a protist cell, an animal cell, a plant cell, or a fungal cell. In an embodiment, the host cell is a mammalian cell including, but not limited to, 293E, CHO, COS, NS0, SP2, PER.C6, or a fungal cell, such as Saccharomyces cerevisiae, or an insect cell, such as Sf9. In an embodiment, two or more binding proteins, e.g., with different specificities, are produced in a single recombinant host cell. For example, the expression of a mixture of antibodies has been called Oligoclonics™ (Merus B. V., The Netherlands). See, e.g., U.S. Pat. Nos. 7,262,028 and 7,429,486. In an embodiment, a method of producing a binding protein disclosed herein comprising culturing any one of the host cells disclosed herein in a culture medium under conditions sufficient to produce the binding protein is provided.

An embodiment provides a composition for the release of a binding protein wherein the composition comprises a crystallized binding protein, an ingredient, and at least one polymeric carrier. In an embodiment, the polymeric carrier is poly (acrylic acid), a poly (cyanoacrylate), a poly (amino acid), a poly (anhydride), a poly (depsipeptide), a poly (ester), poly (lactic acid), poly (lactic-co-glycolic acid) or PLGA, poly (β-hydroxybunyate), poly (caprolactone), poly (dioxanone), poly (ethylene glycol), poly ((hydroxypropyl) methacrylamide, poly [(organo)phosphazene], a poly (ortho ester), poly (vinyl alcohol), poly (vinylpyrrolidone), a maleic anhydride-alkyl vinyl ether copolymer, a pluronic polyol, albumin, alginate, cellulose, a cellulose derivative, collagen, fibrin, gelatin, hyaluronic acid, an oligosaccharide, a glycaminoglycan, a sulfated polysaccharide, or blends and copolymers thereof. In an embodiment, the ingredient is albumin, sucrose, trehalose, lactitol, gelatin, hydroxypropyl-β-cyclodexhn, methoxypolyethylene glycol, or polyethylene glycol.

Another embodiment provides a method for treating a mammal comprising the step of administering to the mammal an effective amount of a composition disclosed herein.

A pharmaceutical composition is also provided, comprising a binding protein disclosed herein and a pharmaceutically acceptable carrier. In a further embodiment, the pharmaceutical composition comprises at least one additional therapeutic agent. For example, the additional agent may be a therapeutic agent for treating a disorder, an imaging agent, a cytotoxic agent, an angiogenesis inhibitor (including but not limited to an anti-V FGF antibody or a VEGF-trap), a kinase inhibitor (including but not limited to a KDR and a TIE-2 inhibitor), a co-stimulation molecule blocker (including but not limited to anti-B7.1, anti-B7.2, CTLA4-1g, anti-CD20), an adhesion molecule blocker (including but not limited to an anti-LFA-1 antibody, an anti-E/L selectin antibody, a small molecule inhibitor), an anti-cytokine antibody or functional fragment thereof (including, but not limited to, an anti-IL-18, an anti-TNF, and an anti-IL-6/cytokine receptor antibody), methotrexate, cyclosporin, rapamycin, FK506, a detectable label or reporter, a TNF antagonist, an anti-rheumatic, a muscle relaxant, a narcotic, a non-steroid anti-inflammatory drug (NSAID), an analgesic, an anesthetic, a sedative, a local anesthetic, a neuromuscular blocker, an antimicrobial, an anti-psoriatic, a cortieosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

Also disclosed, in various embodiments, is a method for treating a human subject suffering from a disorder in which the target, or targets, capable of being bound by a binding protein are detrimental, comprising administering to the human subject a binding protein disclosed herein such that the activity of the target, or targets, in the human subject is inhibited and one or more symptoms is alleviated or treatment is achieved is provided. The binding proteins provided herein can be used to treat humans suffering from autoimmune diseases such as, for example, those associated with inflammation. In an embodiment, the binding proteins provided herein or antigen-binding portions thereof, are used to treat asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, infectious diseases caused by HIV, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, ocular surface inflammatory diseases, or Lyme disease.

### BRIEF DESCRIPTION OF THE DRAWINGS

FIG. 1A shows a bispecific antigen binding protein having the Ambromab configuration.

FIG. 1B shows a mono-specific antigen binding protein having the Ambromab configuration.

FIG. 2A shows the hinge region of a normal heavy chain-light chain pair together with disulfide interaction.

FIG. 2B shows the hinge region of the EPKSC-EPKSA (DA4) Ambromab binding protein including possible disulfide interaction. The arrow points to the cysteine residue that is substituted with alanine in the EPKSC-EPKSA (DA4) Ambromab binding protein

FIG. 2C shows the hinge region of the VE-VE (DA9) Ambromab binding protein, including possible disulfide interaction.

FIG. 2D shows the configuration of 6 Ambromab binding proteins, including the amino acid sequences in the hinge region.

FIG. 2E shows the SEQ ID NOs that correspond with the amino acid sequences depicted in FIG. 2D.

FIG. 3A shows the SEC profile of the Ambromab antigen binding protein with the wild type hinge sequence after protein A purification.

FIG. 3B shows the SEC profile of EPKSC-EPKSA (DA4) Ambromab binding protein after protein A purification.

FIG. 4 shows the stoichiometry of the hinge mutant EPKSC-EPKSA huTNFα Ambromab binding protein via BIAcore analysis.

FIG. 5 shows the results of the L929 neutralization assay for a MAK195.1-containing EPKSC-EPKSA Ambromab binding protein.

FIG. 6 shows non-reducing SDS-PAGE electrophoresis of various monovalent-bispecific, D2E7-GS10-AB420 Ambromab hinge variants.

FIG. 7 shows the non-reduced mass spec profiles of D2E7-GS10-AB420 Ambromab hinge variants.

FIG. 8 shows the SEC data for various D2E7-GS10-AB420 Ambromab hinge variants.

FIG. 9 shows the L929 assay results of various D2E7-GS10-AB420 Ambromab hinge variants.

FIG. 10 shows the pH-sensitive D2E7SS-22 has equal or better potency against human TNF than the parental deimmunized D2E7SS.

FIG. 11 shows non-reduced mass spectroscopy data for DA6-9, and mAb control. The observed molecular weight matched the predicted molecular weight for all Ambromab hinge variants.

FIG. 12 shows the results of an L929 litunan recombinant TNFα (rhTNFα) neutralization assay using anti-TNFα Ambromab binding protein variants DA 4-8.

FIG. 13 shows a summary of the kinetic rate parameters for Ambromab binding protein variants DA 6-9.

FIG. 14 shows the On rate-Off rate map for Ambromab binding protein variants DA 6-9.

FIG. 15 shows the binding of the D2E7SS22-GS10-AB420 Ambromab to TNFα and internalization in dendritic cells.

FIG. 16 shows the different D2E7 monovalent molecules used for pharmacokinetics (PK) studies.

FIG. 17 shows the pharmacokinetics profile of anti-TNFα Ambromab molecules D2E7-GS10-AB420 VE-VE and D2E7SS22-GS10-AB420 VE-VE after 5 mg/kg IV dosing in CD-1 mice.

FIG. 18 shows the anti-TNFα D2E7-GS10-AB420 VE-VE molecule (PR-1603912) serum concentrations after 5 mg/kg IV dosing in CD-1 mice.

FIG. 19 shows the anti-TNFα D2E7-GS10-AB420 VE-VE. Molecule (PR-1603912) serum concentrations after 5 mg/kg IV Dosing in CD-1 mice.

FIG. 20 shows the anti-TNFα D2E7SS22-GS10-AB420 VE-VE molecule (PR-1603915) serum concentrations after 5 mg/kg PV dosing in CD-1 mice.

FIG. 21 shows a summary of the pharmacokinetics of anti-TNFα DVD-like Ambromab molecules DA4, DA5, DA6 and DA8 after 5 mg/kg IV dosing in CD-1 mice.

FIG. 22 shows the pharmacokinetics of anti-TNFα Ambromab molecule DA5 (PR-1614502) and serum concentrations in 5 CD-1 mice after 5 mg/kg IV dosing (W14-0386).

FIG. 23 shows the pharmacokinetics of anti-TNFα Ambromab molecule DA4 (PR-1614502) and serum concentrations in 5 CD-1 mice after 5 mg/kg IV dosing (W14-0386).

FIG. 24 shows the pharmacokinetics of anti-TNFα Ambromab molecule DA6 (PR-1614502) and serum concentrations in 5 CD-1 mice after 5 mg/kg IV dosing (W14-0386).

FIG. 25 shows the pharmacokinetics of anti-TNFα Ambromab molecule DA8 (PR-1614502) and serum concentrations in 5 CD-1 mice after 5 mg/kg IV dosing (W14-0386).

FIG. 26 shows a schematic of the DMPK bio-analysis—anti-TNF capture assay.

FIG. 27 shows a reducing SDS-PAGE gel of both DVD-Ig and mAb versions of 234, 235 QL mutant Ambromab hinge variants. Samples are as follows: 1) DVD EPKSC-EPKSA 2) DVD EPKSC-DKTHT 3) DVD VE-DKTHT 4) DVD VE-VE 5) mAb EPKSC-DKTHT 6) mAb EPKSC-VE 7) mAb VE-VE.

FIG. 28 shows the reducing mass spectrometry profile of D2E7-GS10-420 EPKSC-EPKSA 234 235 QL.

FIG. 29 shows the reducing mass spectrometry profile of D2E7-GS10-420 VE-VE 234 235 QL.

FIG. 30 shows the reducing mass spectrometry profile of D2E7-GS10-420 VE-DKTHT 234 235 QL.

FIG. 31 shows the reducing mass spectrometry profile of D2E7-GS10-420 EPKSC-DKTHT 234 235 QL.

FIG. 32 shows the TOSOH SEC profile of VE-DKTHT 234 235 QL prior to purification.

FIG. 33 shows the TOSOH SEC profile of VE-VE 234 235 QL prior to purification.

FIG. 34 shows the TOSOH SEC profile of EPKSC-DKTHT 234 235 QL prior to purification.

FIG. 35 shows the DVD-Ig versus Ambromab binding protein format.

### DETAILED DESCRIPTION

It is to be understood that this invention is not limited to particular methods and experimental conditions disclosed herein; as such methods and conditions may vary. It is also to be understood that the terminology used herein is for the purpose of describing particular embodiments only, and is not intended to be limiting, since the scope of the present invention will be limited only by the appended claims.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting. All ranges disclosed herein include the endpoints.

The terms “Ambromab binding protein,” “Ambromab immunoglobulin,” “Ambromab antibody,” or “Ambromab Ig” refer to a format of monovalent, multi- or mono-specific therapeutic antibody or immunoglobulin that utilizes mutations (e.g., knobs-into-holes mutations) to promote heterodimerization of two polypeptide chains, each having a heavy chain Fc domain (which can be the same or different Fc sequences on the two chains). A “monovalent” binding protein, such as the Ambromab, is a construct that has only one binding arm, e.g., one set of paired heavy and light chains that form half of the two arms present in a standard antibody format. The monovalent Ambromab binding protein format can comprise one or more antigen binding domains on the single binding arm (e.g., a monovalent construct capable of binding 1, 2, 3, 4, 5, or more different antigens or epitopes on the same antigen).

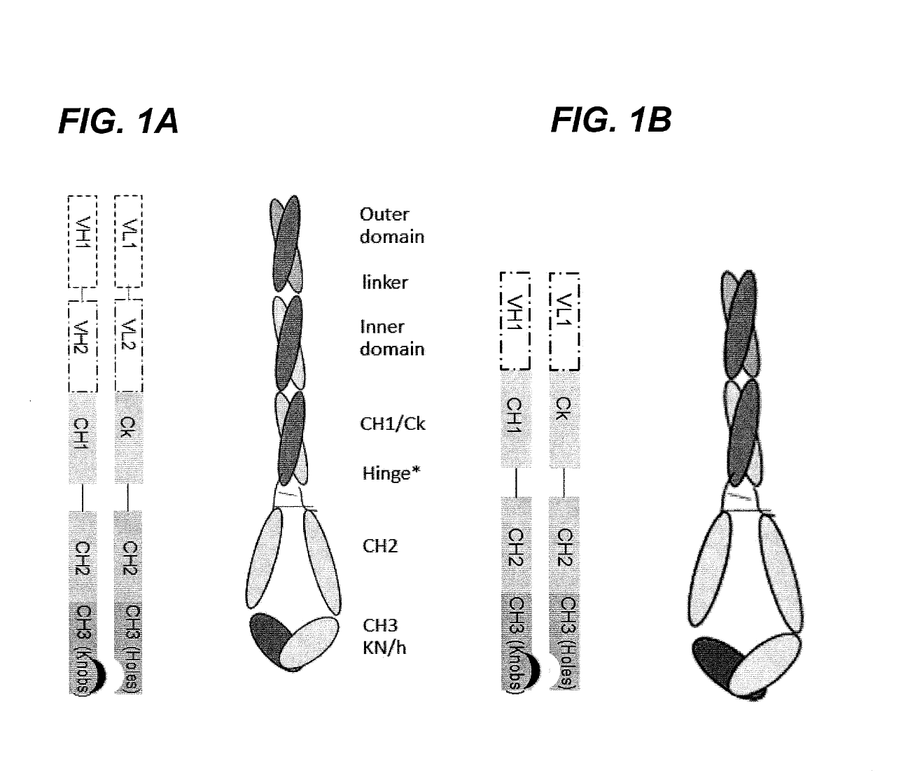

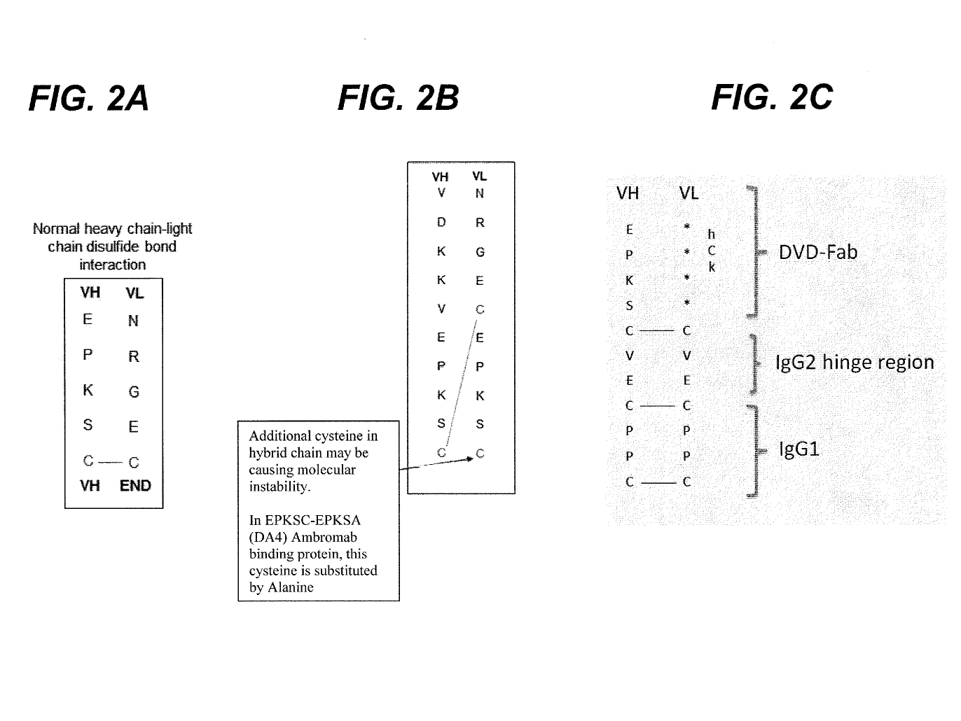

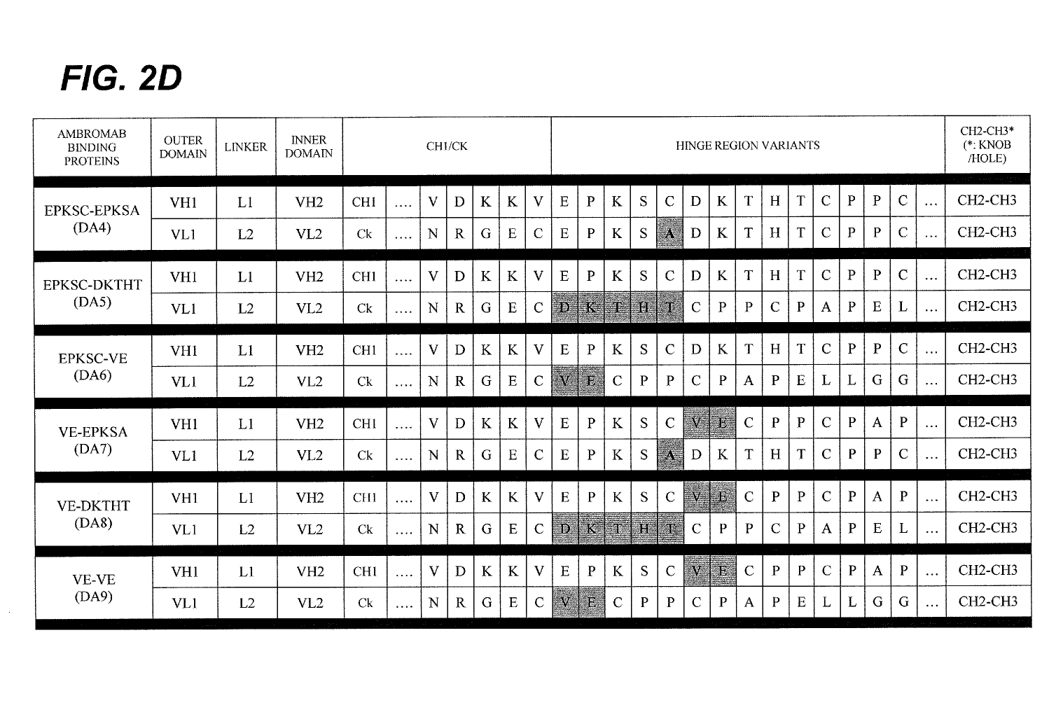

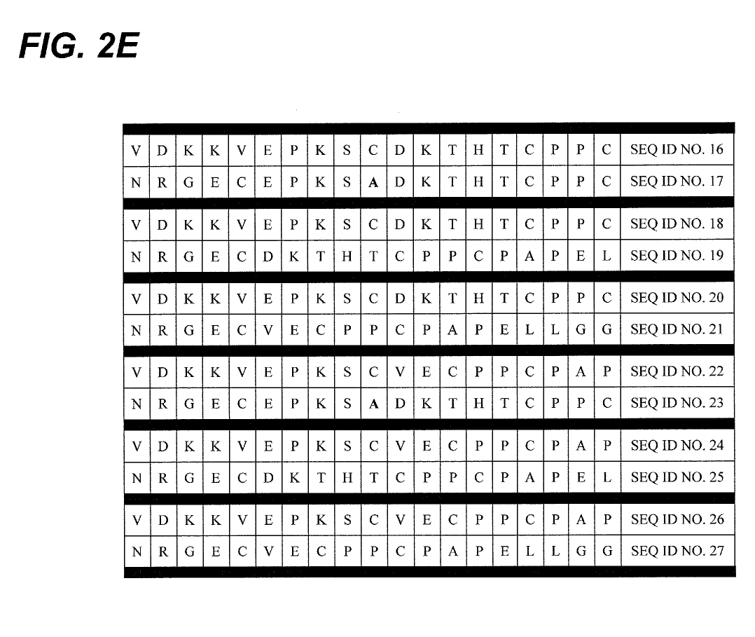

In various embodiments, the Ambromab format comprises one heavy chain that contains a heavy chain Fc, an immunoglobulin hinge (which may comprise a wild-type hinge sequence or a modified variant of a wild-type hinge sequence), a CH1 domain and at least one variable heavy (VH) chain domain. The other heavy chain contains a heavy chain Fc, an immunoglobulin hinge (which may comprise a wild-type hinge sequence or a modified variant of a wild-type hinge sequence), a hCκ or hCλ, and at least one variable light (VL) chain domain (See, for example, FIGS. 1 and 2). In an embodiment, the Ambromab binding protein comprises one heavy chain that contains a heavy chain Fc, an immunoglobulin hinge, a CH1 domain, a second variable heavy (VH2) chain domain, an optional linker, and a first variable heavy (VH1) chain domain, while the other heavy chain contains a heavy chain Fc, a modified immunoglobulin hinge, a hCκ or hCλ, a second variable light (VL2) chain domain, an optional linker, and a first variable light (VL1) chain domain. In an embodiment, the heavy chains comprise knobs-into-holes mutations in the CH3 domains of the first and second polypeptide chains. In some embodiments, the heavy chain and/or light chain variable regions can be derived from a CDR grafted, anti-idiotypic, humanized or parent antibody. Each paired VH/VL variable domain is able to bind to an antigen/ligand. In an embodiment, each VH/VL paired variable domain binds different antigens/ligands or epitopes. In an embodiment, each VH/VL paired variable domain of a bispecific Ambromab binds the same antigen/ligand or epitope. In an embodiment, a bispecific, monovalent Ambromab binding protein has two variable domains with identical specificity and identical variable domain sequences. In an embodiment, a bispecific, monovalent Ambromab binding protein has two variable domains with different specificity and different variable domain sequences. In an embodiment, the Ambromab binding protein may be mono-specific, i.e., capable of binding one antigen, or multi-specific, i.e., capable of binding two or more antigens or epitopes.

The term, “heterodimerization domain” refers to a domain that facilitates the non-covalent association between polypeptide chains. The region of the two polypeptide chains where the two interact, and the structure of that interaction, is the heterodimerization motif. For instance, the heterodimerization region encompasses those amino acids on one polypeptide chain that are within 5 angstroms of an amino acid on the other chain when the two chains are heterodimerized. The specific interaction between those amino acids, e.g., their ionic charge, side chain, and other interactions, define the heterodimerization motif. In certain embodiments, knobs-into-holes mutations may be introduced into these Fc regions to achieve heterodimerization of the Fc regions. See Atwell et al. (1997) J. Mol. Biol. 270:26-35 and U.S. Pat. No. 8,216,805, which are incorporated herein in their entirety. In certain embodiments, the heterodimerization domain may comprise a leucine zipper. See, e.g., U.S. Pat. No. 5,932,446, incorporated in its entirety. Additional dimerization domains are also disclosed in U.S. Pat. No. 5,910,573, incorporated in its entirety.

The term “knobs-into holes mutations” refers to mutations, including those in the CH3 domain of an Fc region, that facilitate heterodimerization of the first and second polypeptide chains in an Ambromab construct. Exemplary mutations useful for this heterodimerization are described in Ridgway et al. (1996) Protein Engin. 9(7):617-21, Atwell et al. (1997) J. Mol. Biol. 270:26-35, and PCT Publication No. WO2014/106015, which are each incorporated by reference herein in their entirety. For instance, electrostatic or hydrophobic interactions can be altered to create knobs and corresponding holes in the two polypeptide chains. For instance, a “protuberance” comprising one or more amino acid modifications may be added to one chain to increase the bulk (e.g., the total volume) taken up by the amino acids. For instance, smaller amino acids can be modified or replaced by those having larger side chains which projects from the interface of the first polypeptide chain (heavy or light chain) and can therefore be positioned in a related cavity in the adjacent second polypeptide chain (light or heavy) so as to stabilize the heterodimer, and thereby favor heterodimer formation over homodimer formation. The protuberance may exist in the original interface or may be introduced synthetically (e.g., by altering one or more nucleic acid encoding the amino acid(s) at the interface). In some embodiments, a protuberance is introduced by modifying the nucleic acid encoding at least one “original” amino acid residue in the interface of the first polypeptide with a nucleic acid encoding at least one “engineered” amino acid residue which has a larger side chain volume than the original amino acid residue. It will be appreciated that there can be more than one original and corresponding engineered residue. The upper limit for the number of original residues which are replaced is the total number of residues in the interface of the first polypeptide.

In addition to a knob (protuberance) added to one chain, a “cavity” (hole) may be added to the second chain, comprising to at least one amino acid side chain which is recessed from the interface of the first or second polypeptide chain (heavy or light chain) and therefore accommodates a corresponding protuberance on the adjacent second polypeptide chain (light or heavy). The cavity may exist in the original interface or may be introduced synthetically (e.g., by altering one or more nucleic acid encoding the amino acid(s) at the interface). In some embodiments, a protuberance is introduced by modifying the nucleic acid encoding at least one “original” amino acid residue in the interface of the first polypeptide with a nucleic acid encoding at least one “engineered” amino acid residue which has a smaller side chain volume than the original amino acid residue. It will be appreciated that there can be more than one original and corresponding engineered residue. The upper limit for the number of original residues which are replaced is the total number of residues in the interface of the first polypeptide.

The term “ligand” refers to any substance capable of binding to, or of being bound by, another substance. Similarly, the term “antigen” refers to any substance to which an antibody may be generated. Although “antigen” is commonly used in reference to a substrate for an antibody, and “ligand” is often used when referring to receptor binding substrates, these terms are not distinguishing one from the other, and encompass a wide range of overlapping chemical entities. For the avoidance of doubt, antigen and ligand are used interchangeably throughout herein. Antigens/ligands may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. In an embodiment of a full-length antibody, each heavy chain is comprised of a heavy chain variable region (VH) and a heavy chain constant region (CH). The heavy chain variable region (domain) is also designated as VH in this disclosure. The CH is comprised of three domains, CH1, CH2 and CH3. Each light chain is comprised of a light chain variable region (VL) and a light chain constant region (CL). The CL is comprised of a single CL domain. The light chain variable region (domain) is also designated as VL in this disclosure. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass. CDR regions can be identified using standard methods, e.g., those of Kabat et al.

The term “CDR-grafted” binding protein refers to an antibody or other binding protein format that comprises heavy and light chain variable region sequences in which the sequences of one or more of the CDR regions of VH and/or VL are replaced with CDR sequences of another binding protein. For example, the two binding proteins can be from different species, such as constructs having murine heavy and light chain variable regions in which one or more of the murine CDRs has been replaced with human CDR sequences.

The term “humanized” binding protein refers to an antibody or other binding protein from a non-human species that has been altered to be more “human-like”, i.e., more similar to human germline sequences. One type of humanized construct is a CDR-grafted antibody or other binding protein, in which non-human CDR sequences are introduced into human VH and VL sequences to replace the corresponding human CDR sequences. A humanized binding protein also encompasses a binding protein or a variant, derivative, analog or fragment thereof that comprises framework region (FR) sequences having substantially (e.g., at least 80%, at least 85%, at least 90%, at least 95%, at least 98% or at least 99% identity to) the amino acid sequence of a human binding protein and at least one CDR having substantially the amino acid sequence of a non-human binding protein. A humanized binding protein may comprise substantially all of at least one, and typically two, variable domains (Fab, Fab′, F(ab′) 2, FabC, Fv) in which the sequence of all or substantially all of the CDR regions correspond to those of a non-human immunoglobulin (e.g., a donor antibody) and the sequence of all or substantially all of the FR regions are those of a human immunoglobulin. The humanized binding protein may also include the human CH1, hinge, CH2, CH3, and/or CH4 regions. In an embodiment, a humanized binding protein also comprises at least a portion of a human immunoglobulin Fc region. In some embodiments, a humanized binding protein only comprises a humanized light chain (i.e., also containing a non-humanized heavy chain). In some embodiments, a humanized antibody only comprises a humanized heavy chain. In some embodiments, a humanized binding protein only comprises a humanized variable domain of a light chain and/or humanized variable domain of a heavy chain. In some embodiments, a humanized binding protein comprises a humanized light chain as well as at least the variable domain of a heavy chain. In some embodiments, a humanized binding protein comprises a humanized heavy chain as well as at least the variable domain of a light chain.

In an embodiment, the C terminal-most amino acid of the light chain variable domain on the first polypeptide is fused to a Cκ light chain constant domain that is linked via a modified hinge region to heavy chain CH2-CH3 constant domains (see FIGS. 2A and 2B). In an embodiment, the C terminal-most amino acid of the heavy chain variable domain on the second polypeptide is fused to a heavy chain CH1 constant domain that is linked via a modified hinge region to heavy chain CH2-CH3 constant domains (see FIGS. 2A and 2B). In some embodiments, the heavy chain CH3 constant domains of the first and second polypeptide chains comprise mutations, e.g., knobs-into-holes mutations, that facilitate proper pairing between the two polypeptide chains (see FIG. 2A).

In an embodiment, each binding site in an Ambromab binding protein comprises a heavy chain variable domain and a light chain variable domain with a total of 6 CDRs involved in antigen binding per antigen binding site. In a specific embodiment of the present invention, at least one binding site comprises a receptor or ligand-binding fragment thereof, capable of binding one or more receptor ligands.

The term “anti-idiotypic” refers to an antibody or other binding protein raised against the amino acid sequence of the antigen combining site of another binding protein. Anti-idiotypic binding proteins may be administered to enhance an immune response against an antigen.

The terms “parent binding protein,” “parent antibody,” or “parent receptor” refer to a pre-existing, or previously isolated binding protein, antibody, or receptor from which a functional binding domain is utilized in a novel binding protein construct.

The term “linker” is used to denote an amino acid residue or a polypeptide comprising two or more amino acid residues joined by peptide bonds and are used to link one or more antigen binding portions. Such linker polypeptides are well known in the art. See, e.g., Holliger et al. (1993) Proc. Natl. Acad. Sci. USA 90:6444-6448; Poljak et al. (1994) Structure 2:1121-1123).

The term “biological activity” refers to any one or more biological properties of a molecule (whether present naturally as found in vivo, or provided or enabled by recombinant means). Biological properties include, but are not limited to, binding a receptor or receptor ligand, inducing cell proliferation, inhibiting cell growth, inducing other cytokines, inducing apoptosis, and enzymatic activity.

The term “neutralizing” refers to counteracting the biological activity of an antigen/ligand when a binding protein specifically binds to the antigen ligand. In an embodiment, the neutralizing binding protein binds to an antigen/ligand (e.g., a cytokine) and reduces its biological activity by at least about 20%, about 40%, about 50%, about 60%, about 70%, about 80%, about 85%, about 90%, about 95% or about 100%, or any percentage in between.

In one embodiment, a TNFα neutralizing antibody or binding protein, or an antibody or binding protein that neutralized hTNFα activity, refers to a construct whose binding to hTNFα results in inhibition of a biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see Example 4). Preferably, the ability of an antibody or other binding protein to neutralize hTNFα activity is assessed by inhibition of hTNFα-induced cytotoxicity of L929 cells. As an additional or alternative parameter of hTNFα activity, the ability of an antibody or other binding protein to inhibit hTNFα-induced expression of ELAM-1 on HUVEC, as a measure of hTNFα-induced cellular activation, can be assessed.

The term “specificity” refers to the ability of a binding protein to selectively bind an antigen/ligand.

The term “affinity” refers to the strength of the interaction between a binding protein and an antigen/ligand, and is determined by the sequence of the binding domain(s) of the binding protein as well as by the nature of the antigen/ligand, such as its size, shape, and/or charge. Binding proteins may be selected for affinities that provide desired therapeutic end-points while minimizing negative side-effects. Affinity may be measured using methods known to one skilled in the art. See, e.g., U.S. Pat. No. 7,612,181.

The term “potency” refers to the ability of a binding protein to achieve a desired effect, and is a measurement of its therapeutic efficacy. Potency may be assessed using methods known to one skilled in the art. See, e.g., U.S. Pat. No. 7,612,181.

The term “cross-reactivity” refers to the ability of a binding protein to bind a target other than that against which it was raised. Generally, a binding protein will bind its target tissue(s)/antigen(s) with an appropriately high affinity, but will display an appropriately low affinity for non-target normal tissues. Individual binding proteins are generally selected to meet two criteria: (1) tissue staining appropriate for the known expression of the antibody target; and (2) similar staining pattern between human and toxic species (mouse and cynomolgus monkey) tissues from the same organ. These and other methods of assessing cross-reactivity are known to one skilled in the art. See, e.g., U.S. Pat. No. 7,612,181.

The term “biological function.” refers the specific in vitro or in vivo actions of a binding protein. Binding proteins may target several classes of antigens/ligands and achieve desired therapeutic outcomes through multiple mechanisms of action. Binding proteins may target soluble proteins, cell surface antigens, as well as extracellular S protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. The in vitro assays and in vivo models used to assess biological function are known to one skilled in the art. See, e.g., U.S. Pat. No. 7,612,181.

The term “stable” refers to a binding protein that retains its physical stability, chemical stability and/or biological activity upon storage. A multivalent binding protein that is stable in vitro at various temperatures for an extended period of time is desirable. Methods of stabilizing binding proteins and assessing their stability at various temperatures are known to one skilled in the art.

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine techniques such as HPLC and other methods known in the art.

Binding proteins may be produced using a variety of host cells or may be produced in vitro, and the relative yield per effort determines the “production efficiency.” Factors influencing production efficiency include, but are not limited to, host cell type (prokaryotic or eukaryotic), choice of expression vector, choice of nucleotide sequence, and methods employed. The materials and methods used in binding protein production, as well as the measurement of production efficiency, are known to one skilled in the art.

The term “immunogenicity” refers to the ability of a substance to induce an immune response. Administration of a therapeutic binding protein may result in a certain incidence of an immune response. Potential elements that might induce immunogenicity in a multivalent format may be analyzed during selection of the parental binding proteins, and steps to reduce such risk can be taken to optimize the parental binding proteins prior to incorporating their sequences into a multivalent binding protein format. Methods of reducing the immunogenicity of antibodies and binding proteins are known to one skilled in the art. See, e.g., U.S. Pat. No. 7,612,181.

The terms “label” and “detectable label” mean a moiety attached to a member of a specific binding pair, such as an antibody, its analyte or an Ambromab binding protein to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; pre-determined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein, such as an antibody or Ambromab, that is chemically linked to a second chemical moiety, such as a therapeutic or cytotoxic agent. The term “agent” includes a chemical compound, a mixture of chemical compounds, a biological macromolecule, or an extract made from biological materials. In an embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The terms “crystal” and “crystallized” refer to a binding protein (e.g., an antibody or Ambromab), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege and Ducruix (1999; 2^{nd} ed.) Crystallization of Nucleic Acids and Proteins, A Practical Approach, pp. 20 1-16, Oxford University Press, N.Y., N.Y.

The term “vector” refers to a nucleic acid molecule capable of transporting another nucleic acid to which it has been linked. One type of vector is a “plasmid,” which refers to a circular double stranded. DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (See U.S. Pat. No. 8,455,219) were used for cloning.

The term “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In an embodiment, host cells include but are not limited to the prokaryotic cell line E. coli; mammalian cell lines CHO, HEK293, COS, NS0, SP2 and PER.C6; the insect cell line Sf9; and the fungal cell Saccharomyces cerevisiae.

The term “transfection” refers to a variety of techniques commonly used for the introduction of exogenous nucleic acid (e.g., DNA) into a host cell, e.g., electroporation, calcium-phosphate precipitation, DEAE-dextran transfection and the like.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “biological sample” refers to a quantity of a substance from a living thing or formerly living thing. Such substances include, but are not limited to, blood, plasma, serum, urine, amniotic fluid, synovial fluid, endothelial cells, leukocytes, monocytes, bone marrow, lymph nodes, spleen, and other cells, organs, and tissues.

The term “component” refers to an element of a composition, in relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include a polypeptide or other analyte as above, that is immobilized on a solid support, such as by binding to an anti-analyte (e.g., anti-polypeptide) antibody. Some components can be in solution or lyophilized for reconstitution for use in an assay.

The term “control” refers to a composition known to not contain analyte (“negative control”) or to contain analyte (“positive control”). A positive control can comprise a known concentration of analyte. A positive control can be used to establish assay performance characteristics and is a useful indicator of the integrity of reagents analytes).

The term “specific binding partner” refers to a member of a specific binding pair. A specific binding pair comprises two different molecules that specifically bind to each other through chemical or physical means. Therefore, in addition to antigen and antibody specific binding, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof; whether isolated or recombinantly produced.

The term “Fc region” refers to the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art. See, e.g., U.S. Pat. Nos. 5,648,260 and 5,624,821. The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The term “antigen-binding portion” refers to one or more fragments of a binding protein (preferably, an antibody, Ambromab, or a receptor) that retain the ability to specifically bind to an antigen. The antigen-binding portion of a binding protein can be performed by fragments of a full-length antibody, as well as bispecific, dual specific, or multi-specific formats; specifically binding to two or more different antigens. Examples of binding fragments encompassed within the term “antigen-binding portion” of an binding protein include (i) an Fab fragment, a monovalent fragment consisting of the VL, VH, CL and CH1 domains; (ii) an F(ab′)2 fragment; (iii) an Fd fragment consisting of the VH and CH1 domains; (iv) an Fv fragment consisting of the VL and VH domains of a single arm of an antibody, (v) a dAb fragment, which comprises a single variable domain; and (vi) an isolated complementarity determining region (CDR). Furthermore, although the two domains of the Fv fragment, VL and VH, encoded by separate genes, they can be joined, using recombinant methods, by a synthetic linker that enables them to be made as a single protein chain in which the VL and VH regions pair to form monovalent molecules (known as single chain FV (scFv). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH-CH1-VH-CH1) which, together with complementary light chain polypeptides, form a pair of antigen binding regions.

The term “multi-specific binding protein” refers to a binding protein capable of binding two or more related or unrelated targets. In an embodiment, a monovalent binding protein may be multispecific in that it possesses one binding domain for each of the different target antigens.

The term “immunoglobulin hinge region” refers to polypeptide sequence comprising at least two consecutive amino acids taken from the sequence of a heavy chain molecule that joins the CH1 domain to the CH2 domain, e.g., in an IgG immunoglobulin. Hinge regions can be subdivided into three distinct domains: upper, middle, and lower hinge domains (Roux et al. (1998) J. Immunol. 161: 4083). In an embodiment, the “immunoglobulin hinge region” comprises the amino acid sequence EPKSCDKTHT.

The terms “Kabat numbering”, “Kabat definitions” and “Kabat labeling” refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “CDR” refers to a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” refers to a region of an antigen that is bound by a binding protein, e.g., a polypeptide and/or other determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (one prevents the binding or modulating effect of the other). In addition, structural definitions of epitopes (overlapping, similar, identical) are informative; and functional definitions encompass structural (binding) and functional (modulation, competition) parameters. Different regions of proteins may perform different functions. For example specific regions of a cytokine interact with its cytokine receptor to bring about receptor activation whereas other regions of the protein may be required for stabilizing the cytokine. To abrogate the negative effects of cytokine signaling, the cytokine may be targeted with a binding protein that binds specifically to the receptor interacting region(s), thereby preventing the binding of its receptor. Alternatively, a binding protein may target the regions responsible for cytokine stabilization, thereby designating the protein for degradation. The methods of visualizing and modeling epitope recognition are known to one skilled in the art. See, e.g., U.S. Pat. No. 7,612,181.

The term “pharmacokinetics” refers to the process by which a drug is absorbed, distributed, metabolized, and excreted by an organism. To generate a multivalent binding protein molecule with a desired pharmacokinetic profile, parent binding proteins with similarly desired pharmacokinetic profiles are selected. The PK profiles of the selected parental binding proteins can be easily determined in rodents using methods known to one skilled in the art. See, e.g., U.S. Pat. No. 7,612,181.

The term “bioavailability” refers to the amount of active drug that reaches its target following administration. Bioavailability is function of several of the previously described properties, including stability, solubility immunogenicity and pharmacokinetics, and can be assessed using methods known to one skilled in the art. See, e.g., U.S. Pat. No. 7,612,181.

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jönsson et al. (1993) Ann. Biol. Clin. 51:19-26. The term “Kon” means the on rate constant for association of a binding protein (e.g., an antibody or Ambromab-Ig) to the antigen to form the, e.g., Ambromab-Ig/antigen complex. The term “Kon” also means “association rate constant”, or “ka”, as is used interchangeably herein. This value indicating the binding rate of a binding protein to its target antigen or the rate of complex formation between a binding protein, e.g., an antibody, and antigen also is shown by the equation below:

Antibody(“Ab”)+Antigen (“Ag”)→Ab−Ag

The term “K_{off}” means the off rate constant for dissociation, or “dissociation rate constant”, of a binding protein (e.g., an antibody or Ambromab-Ig) from the, e.g., Ambromab-Ig/antigen complex as is known in the art. This value indicates the dissociation rate of a binding protein, e.g., an antibody, from its target antigen or separation of Ab−Ag complex over time into free antibody and antigen as shown by the equation below:

Ab+Ag←Ab−Ag

The terms “Kd” and “equilibrium dissociation constant” means the value obtained in a titration measurement at equilibrium, or by dividing the dissociation rate constant (Koff) by the association rate constant (Kon). The association rate constant, the dissociation rate constant and the equilibrium dissociation constant, are used to represent the binding affinity of a binding protein (e.g., an antibody or Ambromab Ig) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The term “variant” means a polypeptide that differs from a given polypeptide in amino acid sequence by the addition (e.g., insertion), deletion, or conservative substitution of amino acids, but that retains the biological activity of the given polypeptide (e.g., a variant IL-17 antibody can compete with anti-IL-17 antibody for binding to IL-17). A conservative substitution of an amino acid, i.e., replacing an amino acid with a different amino acid or amino acids of similar properties (e.g., hydrophilicity and degree and distribution of charged regions) is recognized in the art as typically involving a minor change. These minor changes can be identified, in part, by considering the hydropathic index of amino acids, as understood in the art. See, e.g., Kyte et al. (1982) J. Mol. Biol. 157: 105-132. The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. See, e.g., U.S. Pat. No. 4,554,101. Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptide or fragment thereof that has been differentially processed, such as by proteolysis, phosphorylation, or other post-translational modification, yet retains its biological activity or antigen reactivity, e.g., the ability to bind to IL-17. The term “variant” encompasses fragments of a variant unless otherwise defined. A variant may be 99%, 98%, 97%, 96%, 95%, 94%, 93%, 92%, 91%, 90%, 89%, 88%, 87%, 86%,85%, 84%, 83%, 82%, 81%, 80%, 79%, 78%, 77%, 76%, or 75% identical to the wild type sequence.

Disclosed herein, in various embodiments, are Ambromab binding proteins and methods of making the same. The binding protein can be generated using various techniques. Exemplary expression vectors, host cells and methods of generating the binding proteins are provided in this disclosure.

The antigen-binding variable domains of the binding proteins of this disclosure can be obtained from parent binding proteins, including polyclonal Abs, monoclonal Abs, and/or receptors capable of binding antigens of interest. These parent binding proteins may be naturally occurring or may be generated by recombinant technology. The person of ordinary skill in the art is familiar with many methods for producing antibodies and/or isolated receptors, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanized antibodies. See, e.g., U.S. Pat. No. 7,612,181. Variable domains may also be prepared using CDR grafting and/or affinity maturation techniques. The binding variable domains of the binding proteins can also be obtained from isolated receptor molecules obtained by extraction procedures known in the art (e.g., using solvents, detergents, and/or affinity purifications), or determined by biophysical methods known in the art (e.g., X-ray crystallography, NMR, interferometry, and/or computer modeling).

An embodiment is provided comprising selecting parent binding proteins with at least one or more properties desired in the binding protein molecule. In an embodiment, the desired property is one or more of those used to characterize antibody parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding. See, e.g., U.S. Pat. No. 7,612,181.

The binding proteins disclosed herein may also be designed such that one or more of the antigen binding domains are rendered non-functional. The variable domains may be obtained using recombinant DNA techniques from parent binding proteins generated by any one of the methods described herein. In an embodiment, a variable domain is a murine heavy or light chain variable domain. In an embodiment, a variable domain is a CDR grafted or a humanized variable heavy or light chain domain. In an embodiment, a variable domain is a human heavy or light chain variable domain.

A linker sequence may be present or absent on either or both of the first and second polypeptide chains, and if present, may comprise a single amino acid or a polypeptide sequence. In an embodiment, the choice of linker sequences is based on crystal structure analysis of several Fab molecules. There is a natural flexible linkage between the variable domain and the CH1/CL constant domain in Fab or antibody molecular structure. This natural linkage may contain approximately 10-12 amino acid residues, contributed by 4-6 residues from the C-terminus of a V domain and 4-6 residues from the N-terminus of a CL/CH1 domain. The binding proteins may be generated using N-terminal 5-6 amino acid residues, or 11-12 amino acid residues, of CL or CH1 as a linker in the light chain and heavy chains, respectively. The N-terminal residues of CL or CH1 domains, particularly the first 5-6 amino acid residues, can adopt a loop conformation without strong secondary structures, and therefore can act as flexible linkers between the two variable domains. The N-terminal residues of CL or domains are natural extension of the variable domains, as they are part of the Ig sequences, and therefore their use may minimize to a large extent any immunogenicity potentially arising from the linkers and junctions.

Other linker sequences may include any sequence of any length of a CL/CH1 domain but not all residues of a CL/CH1 domain; for example the first 5-12 amino acid residues of a CL/CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); G/S based sequences (e.g., G4S repeats); hinge region-derived sequences; and other natural sequences from other proteins.

In an embodiment, one or more constant domains are linked to the variable domains using recombinant DNA techniques. In an embodiment, a sequence comprising one or more heavy chain variable domains is linked to a heavy chain constant domain and a sequence comprising one or more light chain variable domains is linked to a light chain constant domain. In an embodiment, the constant domains are human heavy chain constant domains and human light chain constant domains, respectively. In an embodiment, the heavy chain is further linked to an Fc region. The Fc region may be a native sequence Fc region or a variant Fc region. In an embodiment, the Fc region is a human Fc region. In an embodiment, the Fc region includes be region from IgG1, IgG2, IgG3, IgG4, IgA, IgM, IgE, or IgD.

Detailed description of specific binding proteins capable of binding specific targets, and methods of making the same, is provided in the Examples section below.

In one embodiment, at least 50%, at least 75%, at least 90%, at least 95%, at least 99%, or 100% of the assembled immunoglobulin molecules expressed in a host cell are the desired Ambromab binding proteins, and therefore possess enhanced commercial utility.

In an embodiment, the first and second polypeptide chains ofAmbromab binding proteins are expressed in multiple cells, or in a single cell, where the desired Ambromab is at least 50%, at least 75%, at least 90%, at least 95%, at least 99%, or 100% of the assembled immunoglobulin molecules expressed in the host cell(s).

In an embodiment, the binding proteins provided herein are capable of neutralizing the activity of their antigen targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In an embodiment, a method for reducing antigen activity in a subject suffering from a disease or disorder in which the antigen activity is detrimental is provided. A binding protein provided herein can be administered to a human subject for therapeutic purposes.

A disorder in which antigen activity is detrimental refers to diseases and other disorders in which the presence of the antigen in a subject has been shown to be or is suspected of being either responsible for the pathophysiology of the disorder or a factor that contributes to a worsening of the disorder. Accordingly, a disorder in which antigen activity is detrimental is a disorder in which reduction or other alteration of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins.

Additionally, the binding proteins provided herein can be employed for tissue-specific delivery (target a tissue marker and a disease mediator for enhanced local PK thus higher efficacy and/or lower toxicity), including intracellular delivery (targeting an internalizing receptor and an intracellular molecule), delivering to inside brain (targeting transferrin receptor and a CNS disease mediator for crossing the blood-brain barrier). The binding proteins can also serve as a carrier protein to deliver an antigen to a specific location via binding to a non-neutralizing epitope of that antigen and also to increase the half-life of the antigen. Furthermore, the binding proteins can be designed to either be physically linked to medical devices implanted into patients or target these medical devices (See Burke et al. (2006) Advanced Drug Deliv. Rev. 58(3): 437-446; Hildebrand et al. (2006) Surface and Coatings Technol. 200(22-23): 6318-6324; Drug/device combinations for local drug therapies and infection prophylaxis, Wu (2006) Biomaterials 27(11Y⁻2450-2467: Mediation of the cytokine network in the implantation of orthopedic devices, Marques (2005) Biodegradable Systems in Tissue Engineer. Regen. Med. 377-397). Directing appropriate types of cell to the site of medical implant may promote healing and restoring normal tissue function. Alternatively, inhibition of mediators (including but not limited to cytokines), released upon device implantation by a receptor antibody fusion protein coupled to or target to a device is also provided.

Binding protein molecules provided herein are useful as therapeutic molecules to treat various diseases, e.g., wherein the targets that are recognized by the binding proteins are detrimental. Such binding proteins may bind one or more targets involved in a specific disease. In an embodiment, the disorder or condition to be treated comprises the symptoms caused by viral infection in a human which is caused by, for example, HIV, the human rhinovirus, an enterovirus, a coronavirus, a herpes virus, an influenza virus, a parainfluenza virus, a respiratory syncytial virus or an adenovirus.

The binding proteins provided herein can be used to treat neurological disorders. In an embodiment, the binding proteins provided herein, or antigen-binding portions thereof, are used to treat neurodegenerative diseases and conditions involving neuronal regeneration and spinal cord injury.

In an embodiment, diseases that can be treated or diagnosed with the compositions and methods disclosed herein include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

Another embodiment provides for the use of the binding protein in the treatment of a disease or disorder, or the preparation of a medicament for use in the treatment of the disorder. In various embodiments, the disease or disorder is rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, Addison's disease, sporadic, polyglandular deficiency type I and polyglandular deficiency type II, Schmidt's syndrome, adult (acute) respiratory distress syndrome, alopecia, alopecia areata, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-L,K,M antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropenia, renal disease NOS, glomerulonephritides, microscopic vasculitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, drug-induced hepatitis, non-alcoholic steatohepatitis, allergy and asthma, group B streptococci (GBS) infection, mental disorders, depression, schizophrenia, Th2 Type and Th1 Type mediated diseases, acute and chronic pain, different forms of pain, cancers, lung cancer, breast cancer, stomach cancer, bladder cancer, colon cancer, pancreatic cancer, ovarian cancer, prostate cancer, rectal cancer, hematopoietic malignancies, leukemia, lymphoma, Abetalipoprotemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetes mellitus, diabetic aterioselerotic disease, diffuse Lewy body disease, dilated congestive cardiomyopathy, disorders of the basal ganglia, Down's syndrome in middle age, drug-induced movement disorders induced by drugs which block CNS dopamine receptors, drug sensitivity, eczema, encephalomyelitis, endocarditis, endocrinopathy, epiglottitis, Epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis A, His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic: neuritis, isehemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignant lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple mycloma, multiple systems degenerations (Mencel Dejerine-Thomas Shi-Drager and Machado-Joseph), mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-Hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranucleo palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, syncope, syphilis of the cardiovascular system, systemic anaphalaxis, systemic inflammatory response syndrome, systemic onset juvenile rheumatoid arthritis, T-cell or FAB ALL telangiectasia, thromboangitis obliterans, thrombocytopenia, toxicity, transplants, trauma/hemorrhage, type III hypersensitivity reactions, type IV hypersensitivity, unstable angina, uremia, urosepsis, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute isehemia, adult Still's disease, anaphylaxis, anti-phospholipid antibody syndrome, aplastic anemia, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, childhood onset psychiatric disorder, dacryocystitis, dermatomyositis, diabetic retinopathy, disk herniation, disk prolaps, drug induced immune hemolytic anemia, endometriosis, endophthalmitis, episcleritis, erythema multiforme, erythema multiforme major, gestational pemphigoid, Guillain-Barré syndrome (GBS), Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, pauciarticular JRA, peripheral artery occlusive disease (PAOD), peripheral vascular disease (PVD), peripheral artery, disease (PAD), phlebitis, polyarteritis nodosa (or periarteritis nodosa), polychondritis, poliosis, polyarticular JRA, polyendocrine deficiency syndrome, polymyositis, polymyalgia rheumatica (PMR), primary Parkinsonism, prostatitis, pure red cell aplasia, primary adrenal insufficiency, recurrent neuromyelitis optica, restenosis, rheumatic heart disease, sapho (synovitis, acne, pustulosis, hyperostosis, and osteitis), secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), temporal arteritis, toxoplasmic retinitis, toxic epidermal necrolysis, transverse myelitis, TRAPS (tumor necrosis factor receptor, type I allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, or wound healing.

In an embodiment, the binding proteins, or antigen-binding portions thereof, are used to treat cancer or are used in the prevention of cancer, or the inhibition of metastases from the tumors, either when used alone or in combination with radiotherapy and/or chemotherapeutic agents.

Also disclosed herein are pharmaceutical compositions comprising an Ambromab binding protein. In another aspect, methods of treating a patient suffering from a disorder comprise the step of administering any one of the binding proteins disclosed herein, or a pharmaceutical composition comprising the binding protein, before, concurrently, and/or after the administration of a second agent. In an embodiment, the second agent is one or more of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an. anti-IL-1β mAbs, an anti-IL-6 or IL-6 receptor mAb, a growth factor, an elastase inhibitor, a pyridinyl-imidazole compound, an antibody or agonist of TNF, LT, IL-1, IL-2, IL-6, IL-7, IL-8, IL-12, IL-13, IL-15, IL-16, IL-18, IL-23 EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1RI, sIL-1RII, sIL-6R, an antiinflammatory cytokine, IL-4, IL-10, IL11, IL-13, and TGFβ. In a particular embodiment, the pharmaceutical compositions disclosed herein are administered to a patient by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracie, intrauterine, intravesical, bolus, vaginal, rectal, buccal, sublingual, intranasal, or transdermal administration.

Without limiting the disclosure, further information on certain disease conditions is provided.

1) Human Autohnmune and Inflammatory Response

Various cytokines and chemokines have been implicated in general autoimmune and inflammatory responses, including, for example, asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, occular surface inflammatory diseases, or Lyme disease.

In certain embodiments, the binding proteins, or antigen-binding portions thereof, provided herein can be used to treat neurological disorders. In certain embodiments, the binding proteins or antigen-binding portions thereof, provided herein are used to treat neurodegenerative diseases, and conditions involving neuronal regeneration and spinal cord injury.

2) Asthma

Allergic asthma is characterized by the presence of eosinophilia, goblet cell metaplasia, epithelial cell alterations, airway hyperreactivity (AHR), and Th2 and Th1 cytokine expression, as well as elevated serum IgE levels. Corticosteroids are the most important anti-inflammatory treatment for asthma today, however their mechanism of action is non-specific and safety concerns exist, especially in the juvenile patient population. The development of more specific and targeted therapies is therefore warranted.

Various cytokines have been implicated as having a pivotal role in causing pathological responses associated with asthma. The development of mAb against these cytokines as well as Ambromab constructs may prove effective in preventing and/or treating asthma.

Animal models such as an OVA-induced asthma mouse model, where both inflammation and AHR can be assessed, are known in the art and may be used to determine the ability of various binding protein molecules to treat asthma. Animal models for studying asthma are disclosed in Coffman, et al. (2005) J. Exp. Med. 201(12):1875-1879; Lloyd et al. (2001) Adv. Immunol. 77: 263-295; Boyce et al. (2005) J. Exp. Med. 201(12):1869-1873; and Snibson et al. (2005) J. Brit. Soc. Allergy Clin. Immunol. 35(2):146-52. In addition to routine safety assessments of these target pairs specific tests for the degree of immunosuppression may be warranted and helpful in selecting the best target pairs (See Luster et al. (1994) Toxicol. 92(1-3):229-43; Descotes et al. (1992) Dev. Biol. Standard. 77:99-102; Hart et al. (2001) J. Allergy Clin. Immunol. 108(2):250-257).

In various embodiments, the binding proteins disclosed herein can be administered to treat these disorders.

3) Rheumatoid. Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines, chemokines, and growth factors are expressed in diseased joints. Recent studies indicate that the involvement of T cells in RA is mediated to a significant extent by certain cytokines. Beneficial effects of blocking these cytokines were also observed various animal models of the disease (for a review see Witowski et al. (2004) Cell. Mol. Life Sci. 61: 567-579). Whether a binding protein molecule will be useful for the treatment of rheumatoid arthritis can be assessed using pre-clinical animal RA models such as the collagen-induced arthritis mouse model. Other useful models are also well known in the art (See Brand (2005) Comp. Med. 55(2):114-22). Based on the cross-reactivity of the parental antibodies for human and mouse orthologues (e.g., reactivity for human and mouse TNF, human and mouse IL-15, etc.) validation studies in the mouse CIA model may be conducted with “matched surrogate antibody” derived binding protein molecules; briefly, a binding protein based on two (or more) mouse target specific antibodies may be matched to the extent possible to the characteristics of the parental human or humanized antibodies used for human binding protein construction (e.g., similar affinity, similar neutralization potency, similar half-life, etc.).

In various embodiments, the binding proteins disclosed herein can be administered to treat RA.

4) Systemic Lupus Erythematosus (SLE)

The immunopathogenic hallmark of SLE is the polyclonal B cell activation, which leads to hyperglobulinemia, autoantibody production and immune complex formation. Significant increased levels of certain cytokines have been detected in patients with systemic lupus erythematosus (Morimoto et al. (2001) Autoimmunity, 34(1):19-25; Wong et al. (2008) Clin Immunol. 127(3):385-93). Increased cytokine production has been shown in patients with SLE as well as in animals with lupus-like diseases. Animal models have demonstrated that blockade of these cytokines may decrease lupus manifestations (for a review see Nalbandian et al. (2009) 157(2): 209-215). Based on the cross-reactivity of the parental antibodies for human and mouse orthologues (e.g, reactivity for human and mouse CD20, human and mouse interferon alpha, etc.) validation studies in a mouse lupus model may be conducted with “matched surrogate antibody” derived binding protein molecules. Briefly, a binding protein based two (or more) mouse target specific antibodies may be matched to the extent possible to the characteristics of the parental human or humanized antibodies used for human binding protein construction (e.g., similar affinity, similar neutralization potency, similar half-life. etc.).

In various embodiments, the binding proteins disclosed herein can be administered to treat SLE.

5) Multiple Sclerosis

Multiple sclerosis (MS) is a complex human autoimmune-type disease with a predominantly unknown etiology. Immunologic destruction of myelin basic protein (MBP) throughout the nervous system is the major pathology of multiple sclerosis. Of major consideration are immunological mechanisms that contribute to the development of autoimmunity. In particular, antigen expression, cytokine and leukocyte interactions, and regulatory T-cells, which help balance/modulate other T-cells such as Th1 and Th2 cells, are important areas for therapeutic target identification. In MS, increased expression of certain cytokine has been detected both in brain lesions and in mononuclear cells isolated from blood and cerebrospinal fluid. Cells producing these cytokines are highly enriched in active MS lesions, suggesting that neutralization of this cytokine has the potential of being beneficial (for a review see Witowski et al. (2004) Cell. Mol. Life Sci. 61: 567-579).

Several animal models for assessing the usefulness of the binding proteins to treat MS are known in the art (See Steinman et al, (2005) Trends Immunol. 25(11):565-71; Lublin et al. (1985) Springer Semin. Immunopathol.8(3):197-208; Genain et al. (1997) J. Mol. Med. 75(3):187-97; Tuohy et al. (1999) J. Exp. Med. 189(7):1033-42; Owens et al. (1995) Neurol. Clin. 13(1):51-73; and Hart et al. (2005) J. Immunol, 175(7):4761-8). Based on the cross-reactivity of the parental antibodies for human and animal species orthologues validation studies in the mouse EAE model may be conducted with “matched surrogate antibody” derived binding protein molecules. Briefly, a binding protein based on two (or more) mouse target specific antibodies may be matched to the extent possible to the characteristics of the parental human or humanized antibodies used for human binding protein construction (e.g., similar affinity, similar neutralization potency, similar half-life, etc.). The same concept applies to animal models in other non-rodent species, where a “matched surrogate antibody” derived binding protein would be selected for the anticipated pharmacology and possibly safety studies. In addition to routine safety assessments of these target pairs specific tests for the degree of immunosuppression may be warranted and helpful in selecting the best target pairs (See Luster et al. (1994) Toxicol. 92(1-3): 229-43; Descotes et al. (1992) Devel. Biol. Standard. 77: 99-102; Jones (2000) IDrugs 3(4):442-6).

In various embodiments, the binding proteins disclosed herein can be administered to treat these MS.

6) Sepsis

Overwhelming inflammatory and immune responses are essential features of septic shock and play a central part in the pathogenesis of tissue damage, multiple organ failure, and death induced by sepsis. Cytokines have been shown to be mediators of septic shock. These cytokines have a direct toxic effect on tissues; they also activate phospholipase A2. These and other effects lead to increased concentrations of platelet-activating factor, promotion of nitric oxide synthase activity, promotion of tissue infiltration by neutrophils, and promotion of neutrophil activity. The levels of certain cytokines and clinical prognosis of sepsis have been shown to be negatively correlated. Neutralization of antibody or Ambromab constructs against these cytokines may significantly improve the survival rate of patients with sepsis (See Flierl et al. (2008) FASEB J. 22: 2198-2205).

One embodiment pertains to Ambromab constructs capable of binding one or more targets involved in sepsis, such as, for example cytokines, as well as methods of administering such constructs to treat sepsis. The efficacy of such binding proteins for treating sepsis can be assessed in preclinical animal models known in the art (See Buras et al. (2005) Nat. Rev. Drug Discov. 4(10):854-65 and Calandra et al. (2000) Nat. Med. 6(2):164-70).

7) Neurodegenerative Diseases

Neurodegenerative diseases are either chronic in which case they are usually age-dependent or acute (e.g., stroke, traumatic brain injury, spinal cord injury, etc.). They are characterized by progressive loss of neuronal functions (e.g., neuronal cell death, axon loss, neuritic dystrophy, demyelination), loss of mobility and loss of memory). These chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Specific therapies targeting more than one disease mediator may provide even better therapeutic efficacy for chronic neurodegenerative diseases than observed with targeting a single disease mechanism (See Deane et al (2003) Nature Med. 9:907-13; and Masliah et al. (2005) Neuron. 46:857).

In some embodiments, the binding protein molecules provided herein can bind one or more targets involved in chronic neurodegenerative diseases such as Alzheimer's disease and may be administered to treat such a disease. The efficacy of binding protein molecules can be validated in pre-clinical animal models such as the transgenic mice that over-express amyloid precursor protein or RAGE and develop Alzheimer's disease-like symptoms. In addition, binding protein molecules can be constructed and tested for efficacy in the animal models and the best therapeutic binding protein can be selected for testing in human patients. Binding protein molecules can also be employed for treatment of other neurodegenerative diseases such as Parkinson's disease.

8) Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries and the primary injury is usually followed by secondary injury mechanisms (inflammatory mediators e.g., cytokines and chemokines) that worsen the initial injury and result in significant enlargement of the lesion area, sometimes more than 10 fold. Certain cytokine is a mediator of secondary degeneration, which contributes to neuroinflammation and hinders functional recovery.

The efficacy of binding protein molecules can be validated in pre-clinical animal models of spinal cord injury. In addition, these binding protein molecules can be constructed and tested for efficacy in the animal models and the best therapeutic binding protein can be selected for testing in human patients. In general, antibodies do not cross the blood brain barrier (BBB) in an efficient and relevant manner. However, in certain neurologic diseases, e.g., stroke, traumatic brain injury, multiple sclerosis, etc., the BBB may be compromised and allows for increased penetration of binding proteins and antibodies into the brain. In other neurological conditions, where BBB leakage is not occurring, one may employ the targeting of endogenous transport systems, including carrier-mediated transporters such as glucose and amino acid carriers and receptor-mediated transcytosis-mediating cell structures/receptors at the vascular endothelium of the BBB, thus enabling trans-BBB transport of the binding protein. Structures at the BBB enabling such transport include but are not limited to the insulin receptor, transferrin receptor, LRP and RAGE. In addition, strategies enable the use of binding proteins also as shuttles to transport potential drugs into the CNS including low molecular weight drugs, nanoparticles and nucleic acids (Coloma et al. (2000) Pharm Res. 17(3):266-74; Boado et al. (2007) Bioconjug. Chem. 18(2):447-55).

In various embodiments, the binding proteins disclosed herein can be administered to treat these disorders.

9) Oncological Disorders

Monoclonal antibody therapy has emerged as an important therapeutic modality for cancer (von Mehren et al. (2003) Annu. Rev. Med. 54:343-69). Certain cytokines have been suggested to support tumor growth, probably by stimulating angiogenesis or by modulating anti-tumor immunity and tumor growth. Studies indicate that some cytokines may be central to the novel immunoregulatory pathway in which NKT cells suppress tumor immunosurveillance. (For a review see Kolls et al. (2003) Am. J. Respir. Cell Mol. Biol. 28:9-11, and Terabe et al. (,2004) Cancer Immunol Immunother. 53(2):79-85.)

In an embodiment, diseases that can be treated or diagnosed with the compositions and methods provided herein include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarciriorna and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

In an embodiment, the binding proteins provided herein, are capable of binding to one or more antigen associated with cancer. In an embodiment, the binding proteins are administered to treat cancer or in the prevention of metastases from the tumors described herein either when used alone or in combination with radiotherapy and/or other chemotherapeutic agents.

10) Gene Therapy

In a specific embodiment, nucleic acid sequences encoding a binding protein provided herein or another prophylactic or therapeutic agent provided herein are administered to treat, prevent, manage, or ameliorate a disorder or one or more symptoms thereof by way of gene therapy. Gene therapy refers to therapy performed by the administration to a subject of an expressed or expressible nucleic acid. In this embodiment, the nucleic acids produce their encoded binding protein and/or encoded prophylactic or therapeutic agent and thereby mediates a prophylactic or therapeutic effect.

Any of the methods for gene therapy available in the art can be used in the methods provided herein. For general reviews of the methods of gene therapy, see Goldspiel et al. (1993) Clin. Pharmacy 12:488-505; Wu and Wu (1991) Biotherapy 3:87-95; Tolstoshev (1993) Ann. Rev. Pharniacol. Toxicol. 32:573-596; Mulligan (1993) Science 260:926-932; Morgan and Anderson (1993) Ann. Rev. Biochem. 62:191-217; and May (1993) TIBTECH 11(5):155-215. Methods commonly known in the art of recombinant DNA technology which can be used are described in Ausuhel et al. (eds.), Current Protocols in Molecular Biology, John Wiley &Sons, N.Y. (1993); and Kriegler, Gene Transfer and Expression, A Laboratory Manual, Stockton Press, N.Y. (1990). Detailed descriptions of various methods of gene therapy are disclosed in U.S. Patent Publication No. 2005/0042664.

Pharmaceutical Compositions

In various embodiments, pharmaceutical compositions are disclosed comprising one or more of the binding proteins disclosed herein, either alone or in combination with other prophylactic agents, therapeutic agents, and/or pharmaceutically acceptable carriers. The pharmaceutical compositions comprising binding proteins provided herein may be used for, but are not limited to, diagnosing, detecting, or monitoring a disorder, in preventing, treating, managing, or ameliorating a disorder or one or more symptoms thereof, and/or in research. Formulations of pharmaceutical compositions containing one or more of the disclosed binding proteins, either alone or in combination with prophylactic agents, therapeutic agents, and/or pharmaceutically acceptable carriers, are known to one skilled in the art. See, e.g., U.S. Pat. No. 7,612,181.

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). Various formulations of pharmaceutical compositions for specific routes of administration, and the materials and techniques necessary for the various methods of administration, are available.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a pre-determined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. it is to be noted that dosage values may vary with the type and severity of the condition to be alleviated. It is to be further understood that for any particular subject, specific: dosage regimens may be adjusted over time according to the individual need and the professional judgment of the person administering or supervising the administration of the compositions, and that dosage ranges set forth herein are exemplary only and are not intended to limit the scope or practice of the claimed composition.

Combination Therapy

A binding protein provided herein also can also be administered with one or more additional therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein, such as an oncologic agent to complement treatment of a cancer using a binding protein. The combination can also include more than one additional agent, e.g., two, three, or more additional agents.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

Combinations to treat autoimmune and inflammatory diseases include non-steroidal anti-inflammatory drug(s), also referred to as NSAIDS, which include drugs like ibuprofen. Other combinations are corticosteroids including prednisolone; the well-known side-effects of steroid use may be reduced or even eliminated by tapering the steroid dose required when treating patients in combination with the binding proteins provided herein. Non-limiting examples of therapeutic agents the rheumatoid arthritis which can be administered in combination with a binding protein disclosed herein include but are not limited to one or more of the following: cytokine suppressive anti-inflammatory drug(s) (CSAIDs); antibodies to or antagonists of other human cytokines or growth factors, for example, TNF, LT, IL-1, IL-2, IL-3, IL-5, IL-6, IL-7, IL-8, IL-15, IL-16, IL-18, IL-21, IL-23, interferons, EMAP-II, GMM-CSF, FGF, and PDGF. Binding proteins provided herein, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, and CTLA or their ligands, including CD154 (gp39 or CD40L).

Combinations of therapeutic agents may interfere at different points in the autoimmune and subsequent inflammatory cascades. Examples include a binding protein disclosed herein and a TNF antagonist like a chimeric, humanized or human TNF antibody, Adalimumab, (PCT Publication No. WO 97/29131), CA2 (Remicade™), CDP 571, a soluble p55 or p75 TNF receptor, or derivative thereof (p75′TNFR1gG (Enbrel™) or p55TNFR1 gG (Lenercept)), a TNFα converting enzyme (TACE) inhibitor; or an IL-1 inhibitor (an Interleukin-1-converting enzyme inhibitor, IL-IRA, etc.). Other combinations include a binding protein disclosed herein and Interleukin 11. Yet another combination include key players of the autoimmune response which may act parallel to, dependent on or in concert with IL-12 function; especially relevant are IL-18 antagonists including an IL-18 antibody, a soluble IL-18 receptor, or an IL-18 binding protein. It has been shown that IL-12 and IL-18 have overlapping but distinct functions and a combination of antagonists to both may be most effective. Yet another combination is a binding protein disclosed herein and a non-depleting anti-CD4 inhibitor. Yet other combinations include a binding protein disclosed herein and an antagonist of the co-stimulatory pathway CD80 (B7.1) or CD86 (B7.2) including an antibody, a soluble receptor, or an antagonistic ligand.

The binding proteins provided herein may also be combined with an agent, such as methotrexate, 6-MP, azathioprine sulphasalazine, mesalazine, olsalazine chloroquinine/hydroxychloroquine, pencillamine, aurothiomalate (nitrarmiscular and oral), azathioprine, cochicine, a corticosteroid (oral, inhaled and local injection), a beta-2 adrenoreceptor agonist (salbutamol, terbutaline, salmeteral), a xanthine (theophylline, aminophylline), cromoglycate, nedocromil, ketotifen, ipratropium, oxitropium, cyclosporin, FK506, rapamycin, mycophenolate mofetil, leflunomide, an NSAID, for example, ibuprofen, a corticosteroid such as prednisolone, a phosphodiesterase inhibitor, an adenosine agonist, an antithrombotic agent, a complement inhibitor, an adrenergic agent, an agent which interferes with signalling by proinflammatory cytokines such as TNF-α or IL-1 (e.g., IRAK, NIK, IKK p38 or a MAP kinase inhibitor), an IL-1β converting enzyme inhibitor, a TNFα converting enzyme (TACE) inhibitor, a T-cell signaling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivative thereof (e.g., a soluble p55 or p7 S TNF receptor or the derivative p75TNFRIgG (Enbrel™) or p55TNFRIgG (Lenercept), sIL-1R1, SIL-1RII, sIL-6R), an antiinflammatory cytokine (e.g., IL-4, IL-10, IL-11, IL-13 and TGFβ)), celecoxib, folic acid, hydroxychloroquine sulfate, rofecoxib, etanercept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/falmidoxine, acetaminophen, alendronate sodium, prednisolone, morphine sulfate, lidocaine hydrochloride, indomethacin, glucosamine sulfichondroitin, amitriptyline hcl, sulfadiazine, oxycodone hcl/acetaminophen, olopatadine hcl, misoprostol, naproxen sodium, omeprazole, cyclophosphamide, rituxirnab, IL-1 TRAP, MRA, CTLA4-IG, IL-18 BP, anti-IL-18, Anti-IL15, BIRB-796, SCIO-169, VX-702, AMG-548, VX-740, Roflumilast, IC-485, CDC-801, or Mesopram. Combinations include methotrexate car leflunomide and in moderate or severe rheumatoid arthritis cases, cyclosporine.

In one embodiment, the binding protein, or antigen-binding portion thereof, is administered in combination with one of the following agents for the treatment of rheumatoid arthritis: a small molecule inhibitor of KDR, a small molecule inhibitor of Tie-2; methotrexate; prednisone; eelecoxib; folic acid; hydroxychloroquine sulfate; rofecoxib; etariereept; infliximab; leflunomide; naproxen; valdecoxib; sulfasalazine; methylprednisolone; ibuprofen; meloxicam; methylprednisolone acetate; gold sodium thiornalate; aspirin; azathioprine; triamcinolone acetonide; propxyohene napsylate/apap; folate; nabumetone; diclofenac; piroxicam; etodoiac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1, I TRAP; MRA; CTLA4-IG; IL-18 RP; IL-12/23; anti-IL 18; anti-IL 15; BIRB-796; SCIO-469; VX-702; AMG-548; VX 740; Roflumilast; IC-485; CDC-801; and mesopram.

Non-limiting examples of therapeutic agents for inflammatory bowel disease with which a binding protein provided herein can be combined include the following: budenoside; epidermal growth factor; a corticosteroid; cyclosporin, sulfasalazine; aminosalicylates; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1β mAb; an anti-IL-6 mAb; a growth factor; an elastase inhibitor; a midinyl-imidazole compound; an antibody to or antagonist of other human cytokines or growth factors, for example, TNF, LT, IL-1, IL-2, IL-6, IL-7, IL-8, IL15, IL-16, IL-17, IL-18, EMAP-II, GM-CSF, FGF, or PDGF. In some embodiments, binding proteins disclosed herein can be combined with an antibody to a cell surface molecule such as CD2, CD3, CD4, CD8, CD25, CD28, CD30, CD40, CD45, CD69, CD90 or their ligands. The binding protein may also be combined with an agent, such as methotrexate, cyclosporin, FK506, rapamycin, mycophenolate mofetil, leflunomide, an NSAID, for example, ibuprofen, a corticosteroid such as prednisolone, a phosphodiesterase inhibitor, an adenosine agonist, an antithrombotic agent, a complement inhibitor, an adrenergic agent, an agent which interferes with signalling by proinflammatory cytokines such as TNFα, or IL-1 an IRAK, NIK, IKK, p38 or MAP kinase inhibitor), an IL-1β converting enzyme inhibitor, a TNFα converting enzyme inhibitor, a T-cell signalling inhibitor such as a kinase inhibitor, a metalloproteinase inhibitor, sulfasalazine, azathioprine, a 6-mercaptopurine, rrn angiotensin converting enzyme inhibitor, a soluble cytokine receptor or derivative thereof (e.g., a soluble p55 or p75 TNF receptor, sIL-1RI, sIL-1RII, sIL-6R) or an antiinflammatory cytokine (e.g., IL-4, IL-10, IL-11, IL-13 or TGFβ) or a BCL-2 inhibitor.

Examples of therapeutic agents for Crohn's disease in which a binding protein can be combined include the following: a TNF antagonist, for example, an anti-TNF antibody, Adalimumab (PCT Publication No. WO 97/29131; HUMIRA), CA2 (REMICADE), CDP 571, a TNFR-Ig construct, (p75TNFRIgG (ENBREL) or a p55TNFRIgG (LENERCEPT)) inhibitor or a PDE4 inhibitor. In some embodiments, the binding proteins disclosed herein can be combined with a corticosteroid, for example, budenoside and dexamethasone. Binding proteins provided herein or antigen binding portions thereof, may also be combined with an agent such as, sulfas alanine, 5-aminosalicylic acid and olsalazine, or an agent that interferes with the synthesis or action of a proinflammatory cytokine such as IL-1, for example, an IL-1β converting enzyme inhibitor or IL-1ra. The binding proteins disclosed herein or antigen binding portion thereof may also be used with a T cell signaling inhibitor, for example, a tyrosine kinase inhibitor or an 6-mercaptopurine, Binding proteins provided herein, or antigen binding portions thereof, may be combined with IL-11. Binding proteins provided herein, or antigen binding portions thereof, may be combined in a pharmaceutical composition also comprising mesalamine, prednisone, azathioprine, mercaptopurine, methylprednisolone sodium succinate, diphenoxylatelatrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumab or interferon-gamma, or any combination thereof

Non-limiting examples of therapeutic agents for multiple sclerosis with which binding proteins provided herein can be combined include the following: a corticosteroid, prednisolone; methylprednisolone; azathioprine; cyclophosphamide; cyclosporine; methotrexate; 4-aminopyridine; tizanidine; interferon-β1a (AVONEX, Biogen); interferon-β1b (BETASERON; Chiron/Berlex); interferon a-n-3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries. Inc.); hyperbaric oxygen; intravenous in immunoglobulin; clabribine; an antibody to or antagonist of other human cytokines or growth factors and their receptors, for example, TNF, LT, IL-1, IL-2, IL-6, IL-7, IL-8, IL-23, IL-1.5, IL-16, EMAP-II, GM-CSF, FGF, or PDGF. Binding proteins provided herein can be combined with an antibody to a cell surface molecule such as CD2, CD3, CD4, CD8. CD19, CD20, CD25, CD28, CD30, CD40, CD45, CD69, CD80, CD86, CD90 or their ligands. Binding proteins provided herein, may also be combined with an agent, such as methotrexate, cyclosporine, FK506, rapamycin, rnycopheriolate mofetil, leflunomide, an NSAID, for example, ibuprofen, a corticosteroid such as prednisolone, a phosphodiesterase inhibitor, an adensosine agonist, an antithrombotic agent, a complement inhibitor, an adrenergic agent, an agent which interferes with signalling by a proinflammatory cytokine such as TNFα or IL-1 (e.g., IRAK, NIK, IKK, p38 or a MAP kinase inhibitor), an IL-1β converting enzyme inhibitor, a TACE inhibitor, a T-cell signaling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivatives thereof (e.g., a soluble p55 or p75 TNF receptor, sIL-1RI, sIL-1RII, sIL-6R), an antiinflammatory cytokine (e.g., IL-4, IL-10, 11,-13 or TGFβ) or a BCL-2 inhibitor.

Examples of therapeutic agents for multiple sclerosis with which binding proteins provided herein can be combined include interferon-β, for example, IFNβ1a and IFNβ1b; copaxone, corticosteroids, caspase inhibitors, for example inhibitors of caspase-1, IL-1 TNF inhibitors, and antibodies to CD40 ligand and CD80.

Non-limiting examples of therapeutic agents for asthma with which binding proteins provided herein can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, metaproterenol sulfate.

Non-limiting examples of therapeutic agents for COPD with which binding proteins provided herein can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/elavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast, or Roflumilast.

Non-limiting examples of therapeutic agents for psoriasis with which binding proteins provided herein can be combined include the following: small molecule inhibitor of KDR, small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/enroll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, or sulfasalazine.

Examples of therapeutic agents for SLE (Lupus) with which binding proteins provided herein can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. Binding proteins provided herein may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. Binding proteins provided herein may also be used with T cell signaling inhibitors, for example, tyrosine kinase inhibitors; or molecules that target T cell activation molecules, for example. CTLA-4-IgG or anti-B7 family antibodies, anti-PD-1 family antibodies. Binding proteins provided herein, can be combined with IL-11 or anti-cytokine antibodies, for example, fonotolizumab (anti-IFNgamma antibody), or anti-receptor receptor antibodies, for example, anti-IL-6 receptor antibody and antibodies to B-cell surface molecules. Binding proteins provided herein or antigen binding portion thereof may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, Adalimumab (PCT Publication No. WO 97/29131; HUMIRA), CA2 (REMICADE), CDP 571, TNFR-Ig constructs, (p75TNFRIgG (ENBREL) and p55TNFRIgG (LENERCEPT)), or BCL2 inhibitors, because BCL-2 overexpression an transgenic mice has been demonstrated to cause a lupus like phenotype.

The pharmaceutical compositions provided herein may include a “therapeutically effective amount” or a “prophylactically effective amount” of a binding protein provided herein. A “therapeutically effective amount” refers to an amount effective, at dosages and for periods of time necessary, to achieve the desired therapeutic result. A therapeutically effective amount of the binding protein may be determined by a person skilled in the art and may vary according to factors such as the disease state, age, sex, and weight of the individual, and the ability of the binding protein to elicit a desired response in the individual. A therapeutically effective amount is also one in which any toxic or detrimental effects of the binding protein, or antigen binding portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Diagnostics

The disclosure herein also provides diagnostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual.

Anti-idiotype antibodies to the binding proteins disclosed herein are also provided. An anti-idiotype antibody includes as its antigen any protein or peptide-containing molecule that comprises at least a portion of an immunoglobulin molecule such as, but not limited to, at least one complementarily determining region (CDR) of a heavy or light chain or a ligand binding portion thereof, a heavy chain or light chain variable region, a heavy chain or light chain constant region, a framework region, or any portion thereof, that can be incorporated into a binding protein provided herein.

A method of determining the presence, amount or concentration of the target antigen, or fragment thereof, in a test sample is provided. The method comprises assaying the test sample for the antigen, or fragment thereof, by an immunoassay. The immunoassay (i) employs at least one binding protein and at least one detectable label and (ii) comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or fragment thereof, in a control or a calibrator. The calibrator is optionally part of a series of calibrators in which each of the calibrators differs from the other calibrators in the series by the concentration of the antigen, or fragment thereof. The method may comprise (i) contacting the test sample with at least one capture agent, which binds to an epitope on the antigen, or fragment thereof, so as to form a capture agent/antigen, or fragment thereof, complex, (ii) contacting the capture agent/antigen, or fragment thereof, complex with at least one detection agent, which comprises a detectable label and binds to an epitope on the antigen, or fragment thereof, that is not bound by the capture agent, to form a capture agent/antigen, or fragment thereof/detection agent complex, and (iii) determining the presence, amount or concentration of the antigen, or fragment thereof, in the test sample based on the signal generated by the detectable label in the capture agent/antigen, or fragment thereof/detection agent complex formed in (ii), wherein at least one capture agent and/or at least one detection agent is the at least one binding protein.

Alternatively, the method may include (i) contacting the test sample with at least one capture agent, which binds to an epitope on the antigen, or fragment thereof, so as to form a capture agent/antigen, or fragment thereof, complex, and simultaneously or sequentially, in either order, contacting the test sample with detectably labeled antigen, or fragment thereof, which can compete with any antigen, or fragment thereof, in the test sample for binding, to the at least one capture agent, wherein any antigen, or fragment thereof, present in the test sample and the detectably labeled antigen compete with each other to form a capture agent/antigen, or fragment thereof, complex and a capture agent/detectably labeled antigen, or fragment thereof, complex, respectively, and (ii) determining the presence, amount or concentration of the antigen, or fragment thereof, in the test sample based on the signal generated by the detectable label in the capture agent/detectably labeled antigen, or fragment thereof, complex formed in (ii), wherein at least one capture agent is the at least one binding protein and wherein the signal generated by the detectable label in the capture agent/detectably labeled antigen, or fragment thereof, complex is inversely proportional to the amount or concentration of antigen, or fragment thereof, in the test sample.

The test sample may be from a patient, in which case the method may further include diagnosing, prognosticating, or assessing the efficacy of therapeutic/prophylactic treatment of the patient. If the method include assessing the efficacy of therapeutic/prophylactic treatment of the patient, the method optionally further comprises modifying the therapeutic/prophylactic treatment of the patient as needed to improve efficacy. The method may be adapted for use in an automated system or a semi-automated system. Accordingly, the methods described herein also can be used to determine whether or not a subject has or is at risk of developing a given disease, disorder or condition. Specifically, such a method may include the steps of: (a) determining the concentration or amount in a test sample from a subject of analyte, or fragment thereof, (e.g., using the methods described herein, or methods known in the art); and (b) comparing the concentration or amount of analyte, or fragment thereof, determined in step (a) with a pre-determined level, wherein, if the concentration or amount of analyte determined in step (a) is favorable with respect to a pre-determined level, then the subject is determined not to have or be at risk for a given disease, disorder or condition. However, if the concentration or amount of analyte determined in step (a) is unfavorable with respect to the pre-determined level, then the subject is determined to have or be at risk for a given disease, disorder or condition.

Additionally, provided herein is method of monitoring the progression of disease in a subject. Optimally the method may include the steps of: (a) determining the concentration or amount in a test sample from a subject of analyte; (b) determining the concentration or amount in a later test sample from the subject of analyte; and (c) comparing the concentration or amount of analyte as determined in step (b) with the concentration or amount of analyte determined in step (a), wherein if the concentration or amount determined in step (b) is unchanged or is unfavorable when compared to the concentration or amount of analyte determined in step (a), then the disease in the subject is determined to have continued, progressed or worsened. By comparison, if the concentration or amount of analyte as determined in step (b) is favorable when compared to the concentration or amount of analyte as determined in step (a), then the disease in the subject is determined to have discontinued, regressed or improved.

Optionally, the method further comprises comparing the concentration or amount of analyte as determined in step (b), for example, with a pre-determined level, Further, optionally the method comprises treating the subject with one or more pharmaceutical compositions for a period of time if the comparison shows that the concentration or amount of analyte as determined in step (b), for example, is unfavorably altered with respect to the pre-determined level.

1) Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELIS), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure. See, e.g., U.S. Pat. No. 7,612,181.

2) Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or quality control reagents, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art. See, e.g., U.S. Pat. No. 7,612,181.

3) Adaptation of Kit and Method

The kit (or components thereof), well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other suitable platforms available from Abbott Laboratories include, but are not limited to, AxSYM®, IMx® (See, for example, U.S. Pat. No. 5,294,404, PRISM®, EIA (head), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081, 7,419,821, 7,612,181, 7,682,833; and 7,723,099, and U.S. Published Patent Application No. 2004/0018577.

It will be readily apparent to those skilled in the art that other suitable modifications and adaptations of the methods described herein may be obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

The present invention is thither illustrated by the following examples which should not be construed as further limiting.

### Example 1

### Design and Construction of Ambromab Constructs

An Ambromab is a format of monovalent, multi or mono-specific therapeutic binding proteins that utilizes knobs-into-holes mutations to combine two different heavy chain Fcs. In certain embodiments, one heavy chain contains a heavy chain Fc, hinge, and variable heavy region and the other chain contains a heavy chain Fc, modified hinge and hCκ (or hCλ) and light chain variable region (FIG. 1).

Molecules can be monovalent multispecific (FIG. 1A) or monovalent monospecific (FIG. 1B) and can be produced in an efficient manner utilizing standard transfection and purification procedures.

The bispecific molecule is approximately 120 KDa in size, utilizes a heavy and light chain variable region and contains an intact Fc. The format may be useful in implications where a monovalent therapeutic will confer advantages over a bispecific molecule, such as in minimizing immune complex formation, minimizing receptor crosslinking on cell surface targets, minimizing or eliminating avidity effects when this effect is detrimental, conferring antagonistic versus agonistic responses, as well as many other indications where monovalent properties will confer positive effects on the biology of specific targets.

Ambromab format utilizes 2 constructs, versus 3 or 4 for other monovalent molecules, is easily purified on protein A and SEC columns, and can be synthesized by mammalian culture systems. It couples a heavy and light chain with an intact Fc by combining one light chain with one Fc. In addition this hybrid chain utilizes a modified hinge region to ensure that the naturally occurring heavy chain and light chain disulfide bond is not perturbed. Hinge modifications ensure a smooth transition between the 2 chains that allowed for the proper pairing of this chain to a heavy chain. Knobs-into-holes mutations are incorporated into the 2 chains to ensure that heavy chain homodimers are not formed. This coupled with naturally occurring CH1-Ck and VH-VL interactions drive the proper formation of this molecule during its synthesis.

Construction; of Ambromab Molecules

The heavy chain containing a heavy chain Fc, hinge, and variable heavy chain and the other chain containing a heavy chain Fc, modified hinge and hCκ (or hCλ) and light chain variable region were cloned into eukaryotic expression vectors using standard recombinant techniques.

The D2E7 VH outer domain, VH linker and IL-17 (AB420) inner VH domain cDNA sequences were PCR amplified with platinum PCR. SuperMix High Fidelity (Invitrogen, Carlsbad, Calif.) and cloned into an expression vector comprising a CH1 heavy chain constant domain fused in frame to a first modified IgG1 hinge regions fused in frame to human IgG1 CH2 and CH3, where the CH3 domain contains a knobs-into-holes mutation (Atwell et al. (1997) J. Mol, Biol. 270:26-35).

The D2E7 VL outer domain, VL linker and IL-17 (AB420) inner VL domain cDNA sequences were PCR amplified with platinum PCR SuperMix High Fidelity (Invitrogen, Carlsbad, Calif.) and cloned into a second expression vector comprising a Cκ light chain constant domain fused in frame to a second modified IgG1 hinge regions fused in frame to human IgG1 CH2 and CH3, where the CH3 domain contains a knobs-into-holes mutation (Atwell et al. (1997) J. Mol. Hied. 270:26-35).

After preparation, the plasmid sequences were confirmed by the dideoxy chain termination method using an ABI 3730XL Genetic Analyzer (Applied Biosystems, Foster City, Calif.).

The amino acid sequences of the various domains and linkers are shown in Tables 1-7 below. The amino acid sequence and nomenclature of the different Ambromab binding proteins is shown in FIG. 2D. FIGS. 2A, 2B and 2C depict possible disulfide interaction between the VH and VL polypeptide chains of the Ambromab binding proteins having a EPKSC-EPKSA (FIG. 2B) or VE-VE (FIG. 2C) hinge region.

TABLE 1
Amino acid sequences of various VH outer and inner domains

| | SEQ | |
|---|---|---|
| VH outer | ID | |
| domains | NO | Sequence |
| MAK195.1 | [SEQ | EVQLVESGGGLVQPGGSLRLSCAASGFTFSNYGVTWVRQAPGKGLEWVS |
| outer | ID 1] | MIWADGSTHYASSVKGRFTISRDNSKNTLYLQMNSLRAEDTAVYYCARE |
| domain | | WQHGPVAYWGQGTLVTYVSS |
| D2E7 | [SEQ | EVQLVESGGGLVQPGRSLRLSCAASGFTFDDYAMHWVRQAPGKGLEWV |
| outer | ID 2] | SAITWNSGHIDYADSVEGRFTISRDNAKNSLYLQMNSLRAEDIAVYYCA |
| domain | | KVSYLSTASSLDYWGQGTIVTVSS |
| D2E7SS22 | [SEQ | EVQLVESGGGLVQPGRSLRLSCAASGFTFDHYAIVIKWVRQAPGKGLEW |
| outer | ID 3] | VSAITWNSGHIDYADSVEGRFTISRDNAKNSLYLQKNSLRAEDTAVYYC |
| domain | | AKVSYLSTASSLDYWGQGTIVTVSS |
| VH linker | [SEQ | GGGGSGGGGS |
| | ID 4] | |
| AB420 | [SEQ | EVQLVQSGAEVKKPGSSVKVSCKASGGSFGGYGIGVWRQAPGQGLEWM |
| inner | ID 5] | GGITPFFGFADYAQKFQGRVTITADESTTTAYMELSGLTSDDTAVYYCAR |
| domain | | DPNEFWGGYYSTHDFDSWGOGTTVTYSS |

TABLE 2
Amino acid sequences of various VL outer and inner domains

| | SEQ. | |
|---|---|---|
| VL outer | ID | |
| domains | No. | Sequence |
| MAK195.1 | [SEQ | DIQMTQSPSSLSASVGDRVTITCRASQLVSSAVAWVQQKPGKAPKLLIYW |
| outer | ID 6] | ASARHTGVPSRFSGSGSGTDFTLTISSLQPEDFATYYCQQHYKTPFTFGQG |
| domain | | TKLEIKR |
| D2E7 | [SEQ | DIQMTQSPSSLSASVGDRVTITCRASQGIRNYLAWYQQKPGKAPKLLIYA |
| outer | ID 7] | ASTLQSGVPSRESGSGSGTDFTLTISSLQPEDVATYYCQRYNRAPYTFGQG |
| domain | | TKVEIKR |
| D2E7SS22 | [SEQ | DIQMTQSPSSLSASVGDRVTITCRASHSIRNYLSWYQQKPGKAPKLLIYAA |
| outer | ID 8] | STLQSGVPSRFSGSGSGTDFTTISSLQPEDVATYYCQRYNRAPYTFGQGTK |
| domain | | VEIKR |
| VL linker | [SEQ | GGSGGGGSG |
| | ID 9] | |
| AB420 | [SEQ | EIVLTQSPDFQSVTPKEKVTITCRASQDIGSELHWYQQKPDQPPKLLIKYA |
| inner | ID 10] | SHSTSGVPSRFSGSGSGTDFTLTINGLEAEDAGTYYCHQTDSLPYTFGPGT |
| domain | | KVDIKR |

TABLE 3
Amino add sequences of VH hinge regions

| VH hinge | SEQ. ID No. | |
|---|---|---|
| 1 | [SEQ. ID 11] | EPKSCDKTHTCPPC |
| 2 | [SEQ. ID 12] | VECPPC |

TABLE 4
Amino acid sequences of VL hinge regions

| VL hinge | | |
|---|---|---|
| 1 | [SEQ. ID 13] | EPKSADKTHTCPPC |
| 2 | [SEQ. ID 14] | DKTHTCPPC |
| 3 | [SEQ. ID 15] | VECPPC |

TABLE 5
Hinge combinations
Hinge Combinations
1 + 1
1 + 2
1 + 3
2 + 1
2 + 2
2 + 3

TABLE 6
VH domain Fc profiles

| Inner-linker-outer VH | Fc |
|---|---|
| MAK195.1-GS10-AB420 | IgG1 wild type knob-Fc |
| D2E7-GS10-AB4Z0 | IgG1 234 235, QL knob-Fc |
| D2E7SS22-GS10-AB420 | IgG1 234, 235 knob-Fc |

TABLE 7
VL domain Fc profiles

| Inner-linker-outer VL | Fc |
|---|---|
| MAK195.1-GS10-AB420 | IgG1 lwildtype hole-Fc |
| D2E7-GS10-AB420 | IgG1 234 235, QL hole-Fc |
| D2E7SS22-GS10-AB420 | IgG1 234, 235 hole-Fc |

### Example 2

### Transient Expression of Ambromab Molecules in 293 Cells

The bispecific D2E7-GS10-AB420 Ambromab VH plasmid was paired with the corresponding VL, plasmid and transfected into human embryonic kidney 293-6E cells (American Type Culture Collection, Manassas, Va.) with polyethylenimine (Sigma, St. Louis, Mo.). The cell culture media was harvested six to seven days-post transfection and the antibodies were purified using protein G chromatography (Invitrogen, Carlsbad, Calif.) according to the manufacturer's instructions. Most Ambromab binding proteins were expressed well in 293 cells as compared to the expression level of regular antibodies, indicating that these antibodies can be expressed efficiently in mammalian cells. Total yields from 500 ml of supernatant are shown in Table 8 below. Total yields from “gene to protein” are shown in Table 9.

TABLE 8
Total yield of various monvalent-bispecific D2E7-
GS10-IL-17(AB420) Ambromab hinge variants.

| | Total yield from 500 mls | |
|---|---|---|
| Hinge variant | 293 6e supernatant | |
| (VH-VL) | (total mgs; mg/ml) | |
| D2E7-GS10-IL-17(AB420) EPKSC-EPKSA | 0.75 | mg |
| D2E7-GS10-IL-17(AB420) EPKSC-DKTHT | 4.5 | mg |
| D2E7-GS10-IL-17(AB420) VE-DKTHT | 5.22 | mg |
| D2E7-GS10-IL-17(AB420) VE-VE | 10.5 | mg |

TABLE 9
Results of a L929 Human recombinant TNFα (rhTNFα) neutralization
assay using anti-TNFα Ambromab binding protein variants DA 4-8.

| Hinge variant | Total yield from “gene to protein” | |
|---|---|---|
| (VH-VL) | (total mgs; mg/ml) | |
| EPKSC-EPKSA (DA4) | 10.4 | mg |
| EPKSC-DKTHT (DA5) | 15 | mg |
| EPKSC-VE (DA6) | 10.2 | mg |
| VE-EPKSA (DA7) | 9.48 | mg |
| VE-DKTHT (DA8) | 11.1 | mg |
| VE-DKTHT (DA8) | 8.95 | mg |

### Example 3

### Characterization of Ambromab Molecules with SDS-PAGE and Size Exclusion Chromatography (SEC)

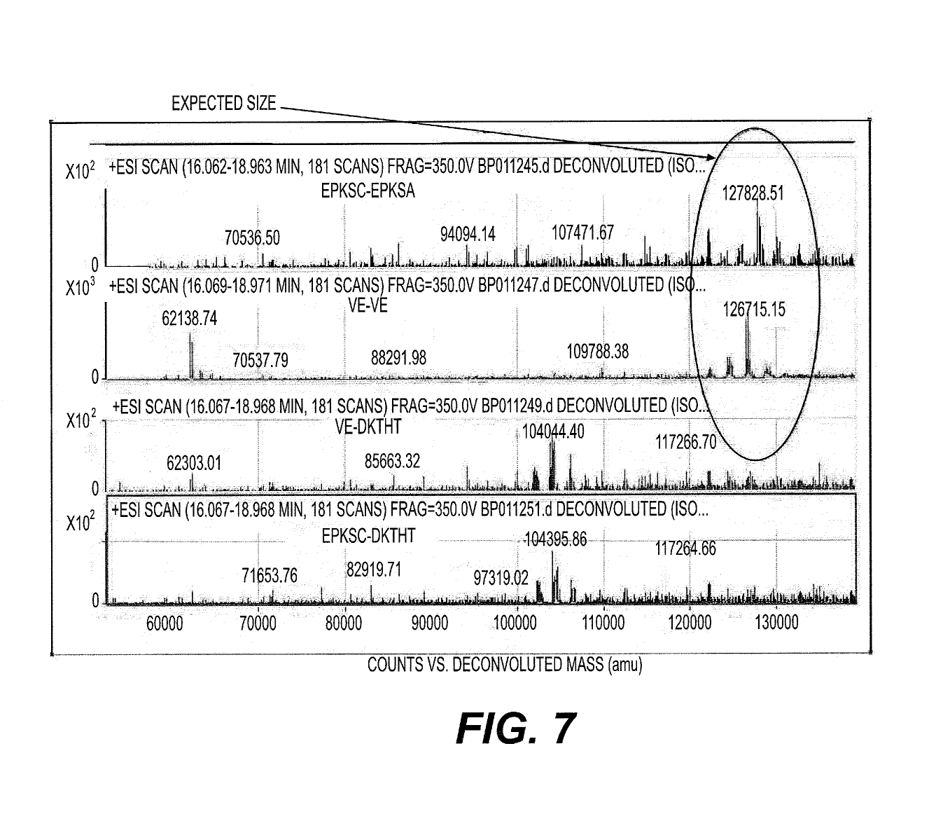

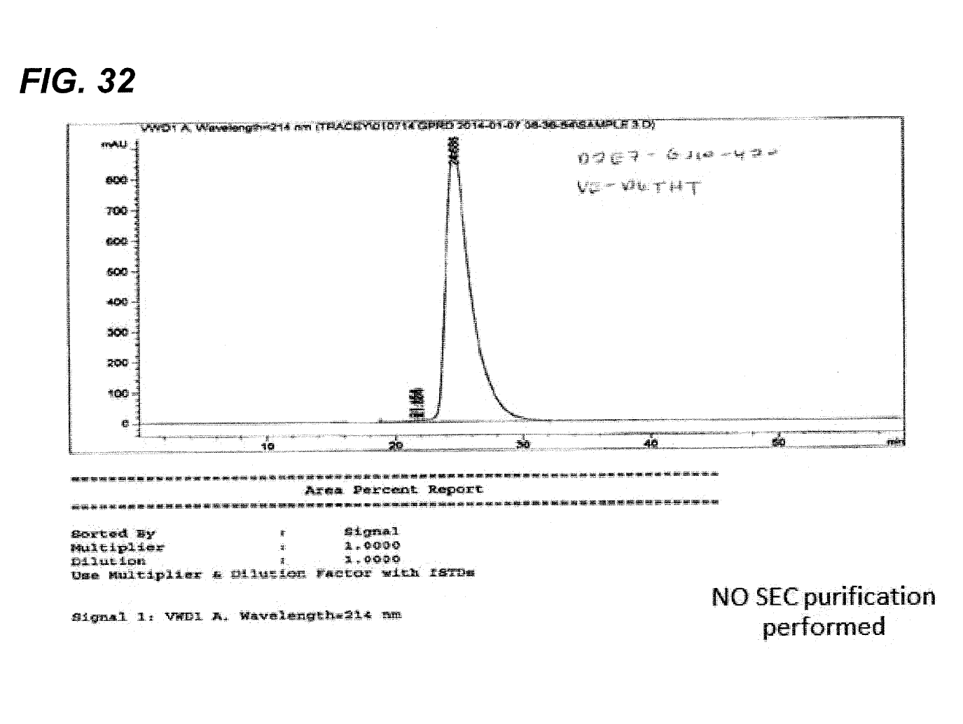

The EPKSC-EPKSA, EPKSC-DKTHT, VE-DKTHT and VE-VE D2E7-GS10-AB420 bispecific Ambromab hinge variants were analyzed by SDS-PAGE under non-reducing conditions. Under non-reducing conditions, each of the protein samples showed a single band (see FIG. 7). SDS-PAGE of different Ambromab variants under reducing conditions is shown in FIG. 32.

SEC analysis was performed to confirm the size of the Ambromab EPKSC-EPKSA D2E7-GS10-AB420 molecule and the Ambromab EPKSC-EPKSC D2E7-GS10-AB420 molecule with an unmodified hinge region. For the analysis, the Ambromab EPKSC-EPKSA protein, in phosphate buffered saline (PBS), was applied to a Superdex 200, 300×10 mm column (GE Healthcare, Piscataway, N.J.). An HPLC instrument, Model 10A (Shimadzu, Columbia, Md.) was used for SEC. All proteins were detected using UV light at 280 nm and 214 nm. The elution was isocratic at a flow rate of 0.5 mL/minute.

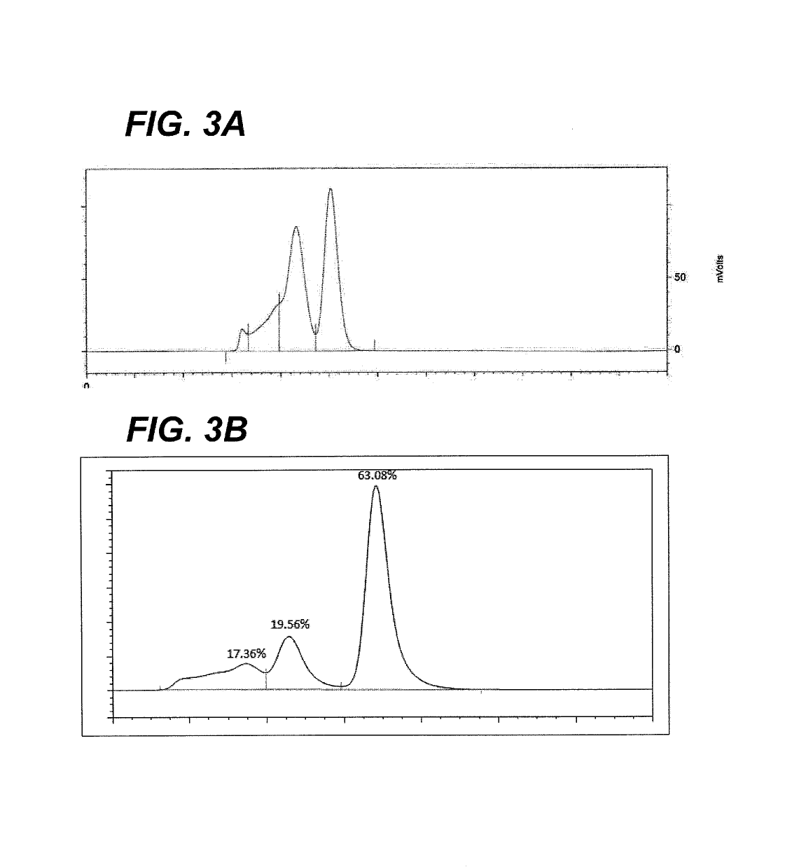

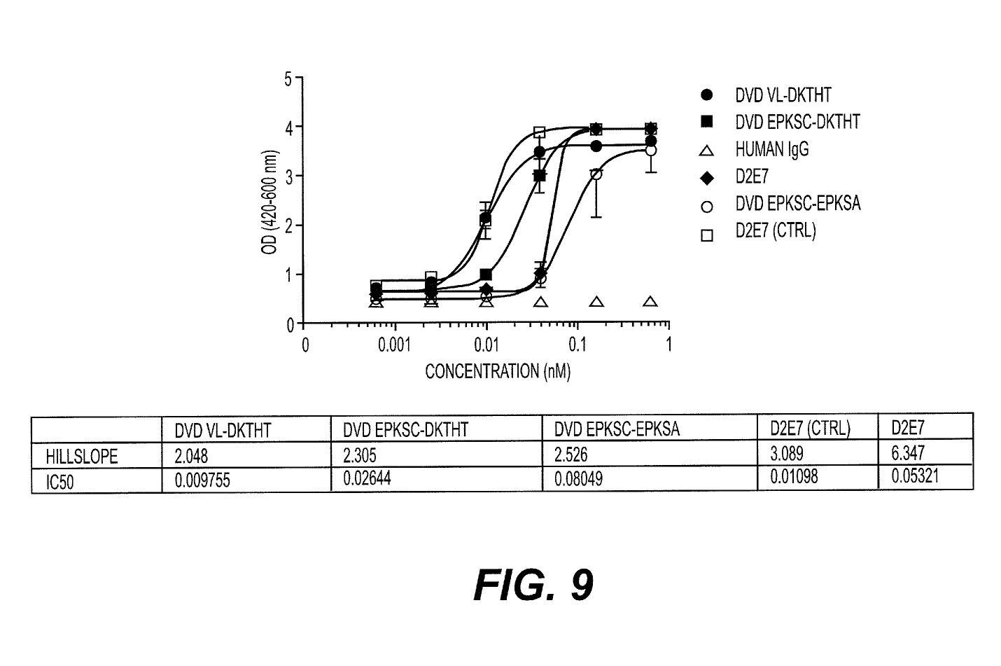

The SEC data in FIG. 3 demonstrated that the Ambromab EPKSC-EPKSC D2E7-GS10-AB420 molecule with an unmodified hinge regions was inherently unstable. Only about 40% of the protein was in monomeric form (see FIG. 3A). In contrast, the SEC profile of EPKSA hybrid chain hinge mutant after protein A purification (see FIG. 3B) improved the percent monomer by 20%. The SEC purified sample was stable under standard conditions. A comparison of the SEC profiles for the EPKSC-EPKSA, EPKSC-DKTHT, VE-DKTHT and VE-VE D2E7-GS10-AB420 bispecific Ambromab molecules can be found in FIG. 9.

The TOSOH SEC profile of VE-DKTHT 234 235 QL prior to purification is shown in FIG. 37. The TOSOH SEC profile of VE-VE 234 235 QL prior to purification is shown in FIG. 38. The TOSOH SEC profile of EPKSC-DKTHT 234 235 QL prior to purification is shown in FIG. 39.

### Example 4

### Biacore Analysis

The BIACORE assay (Biacore, Inc, Piscataway, N.J.) determines the affinity of antibodies or Ambromab binding molecules with kinetic measurements of on-rate and off-rate constants. Binding of antibodies or Ambromab binding molecules to a target antigen (for example, a purified recombinant target antigen) is determined by surface plasmon resonance-based measurements with a Biacore® 1000 or 3000 instrument (Biacore® AB, Uppsala, Sweden) using running HBS-EP (₁₀ mM HEPES [pH 7.4], 150 mM NaCl, 3 mM EDTA, and (1.005% surfactant P20) at 25° C. All chemicals are obtained from Biacore® AB (Uppsala, Sweden) or otherwise from a different source as described in the text. For example, approximately 5000 RU of goat anti-mouse IgG, (Fcγ), fragment specific polyclonal antibody (Pierce Biotechnology Inc, Rockford, Ill.) diluted in 10 mM sodium acetate (pH 4.5) is directly immobilized across a CM5 research grade biosensor chip using a standard amine coupling kit according to manufacturer's instructions and procedures at 25 μg/ml. Unreacted moieties on the biosensor surface are blocked with ethanolamine. Modified carboxymethyl dextran surface in flowcell 2 and 4 is used as a reaction surface. Unmodified carboxymethyl dextran without goat anti-mouse IgG in flow cell 1 and 3 is used as the reference surface. For kinetic analysis, rate equations derived from the 1:1 Langmuir binding model are fitted simultaneously to association and dissociation phases of all eight injections (using global fit analysis) with the use of Bioevaluation 4.0.1 software. Purified antibodies or Ambromab EPKSC-EPKSA D2E7-GS10-AB420 are diluted in HEPES-buffered saline for capture across goat anti-mouse IgG specific reaction surfaces. Antibodies or the Ambromab binding molecule to be captured as a ligand (25 μg/ml) are injected over reaction matrices at a flow rate of 5 μl/minute. The association and dissociation rate constants, kon (M−1 s−1) and koff (s−1) are determined under a continuous flow rate of 25 μl/minute. Rate constants are derived by making kinetic binding measurements at different antigen concentrations ranging from 10-200 nM. The equilibrium dissociation constant (M) of the reaction between antibodies or Ambromab binding molecule and the target antigen is then calculated from the kinetic rate constants by the following formula: KD=koff/kon. Binding is recorded as a function of time and kinetic rate constants are calculated.

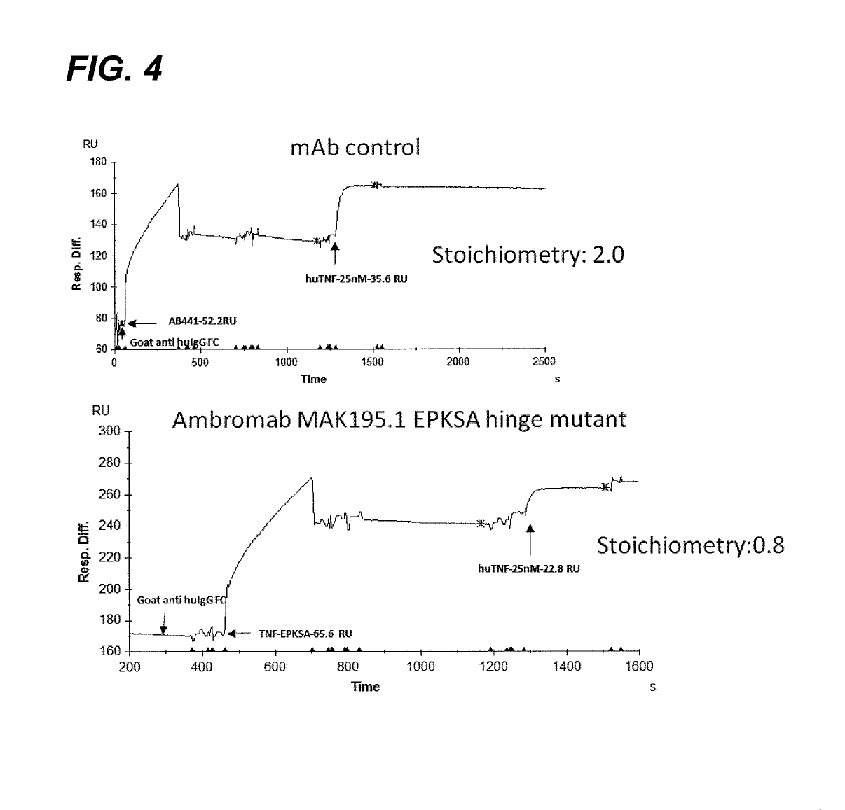

The stoichiometry of the Ambromab EPKSC-EPKSA D2E7-GS10-AB420 with huTNFα is shown in FIG. 4.

### Example 5

### Neutralization of TNFα-Induced Cytotoxicity in L929 Cells

Human recombinant TNFα (rhTNFα) causes cell cytotoxicity to murine L929 cells after an incubation period of 18-24 hours. Human anti-hTNFα antibodies were evaluated in L929 assays by co-incubation of anti-TNFαantibodies (D2E7) or the Ambromab EPKSC-EPKSA D2E7-GS10-AB420 with rhTNFα and the cells as follows.

A 96-well microtiter plate containing 100 μl of anti-hTNFα Abs was serially diluted 1/3 down the plate in duplicates using RPMI medium containing 10% fetal bovine serum (FBS). Fifty microliters of rhTNFα was added for a final concentration of 500 pg/ml in each sample well. The plates were then incubated for 30 minutes at room temperature. Next, 50 μl of TNFα-sensitive L929 mouse fibroblasts cells were added for a final concentration of 5×10⁴ cells per well, including 1 μg/ml Actinomyrin-D. Controls included medium plus cells and rhINFα plus cells. These controls, and a TNFα standard curve, ranging from 2 ng/ml to 8.2 pg/ml, were used to determine the quality of the assay and provide a window of neutralization. The plates were then incubated overnight (18-24 hours) at 37° C. in 5% CO2.

One hundred microliters of medium was removed loin each well and 50 μl of 5 mg/ml 3,(4,4-dimethylthiazol-2-yl)2,5-diphenyl-tetrazolium bromide (MTT; commercially available from Sigma Chemical Co., St. Louis, Mo.) in PBS was added. The plates were then incubated for 4 hours at 37° C. Fifty microliters of 20% sodium dodecyl sulfate (SDS) was then added to each well and the plates were incubated overnight at 37° C. The optical density at 420/600 nm was measured, curves were plotted for each sample and IC50s were determined by standard methods,

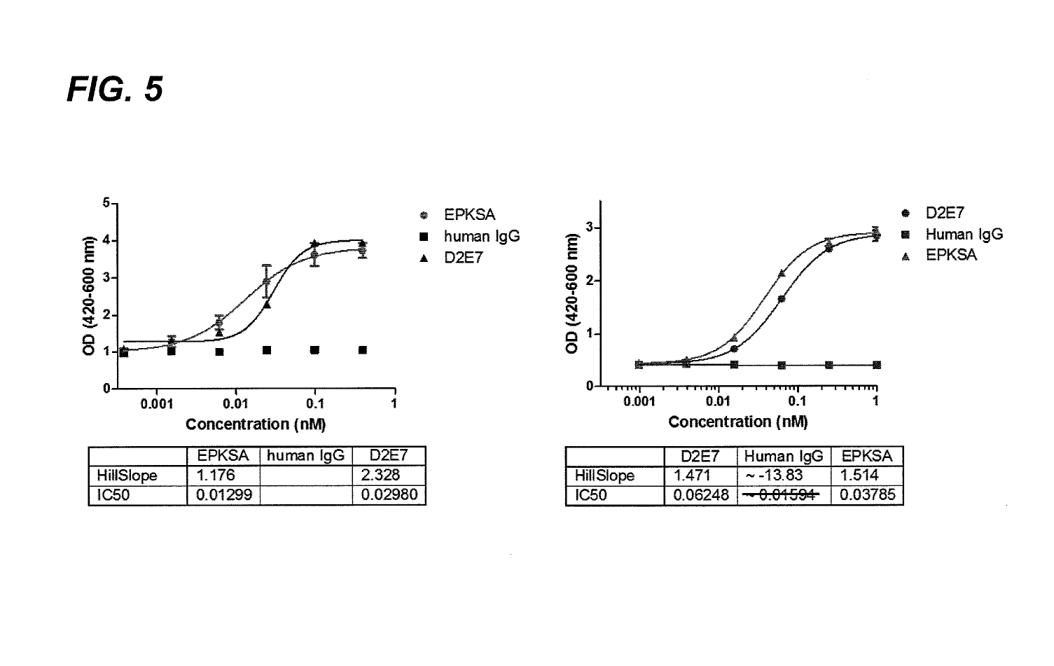

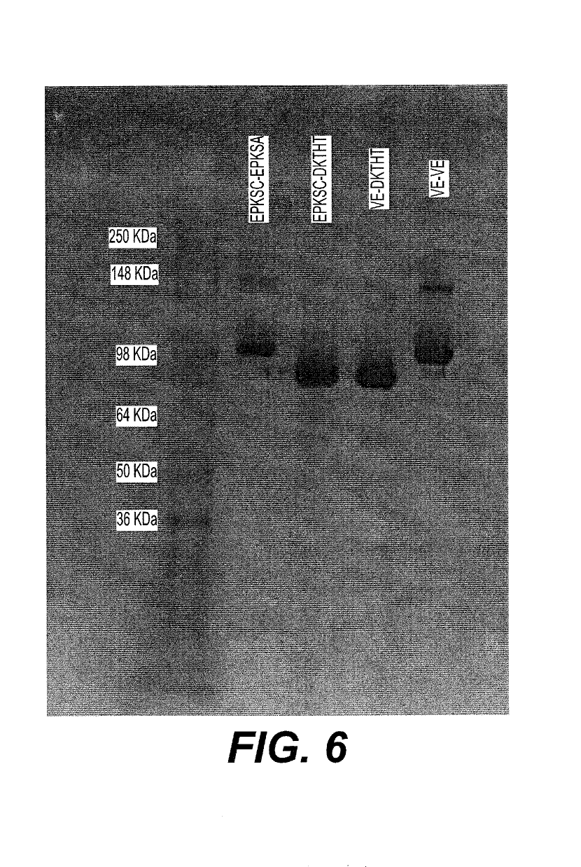

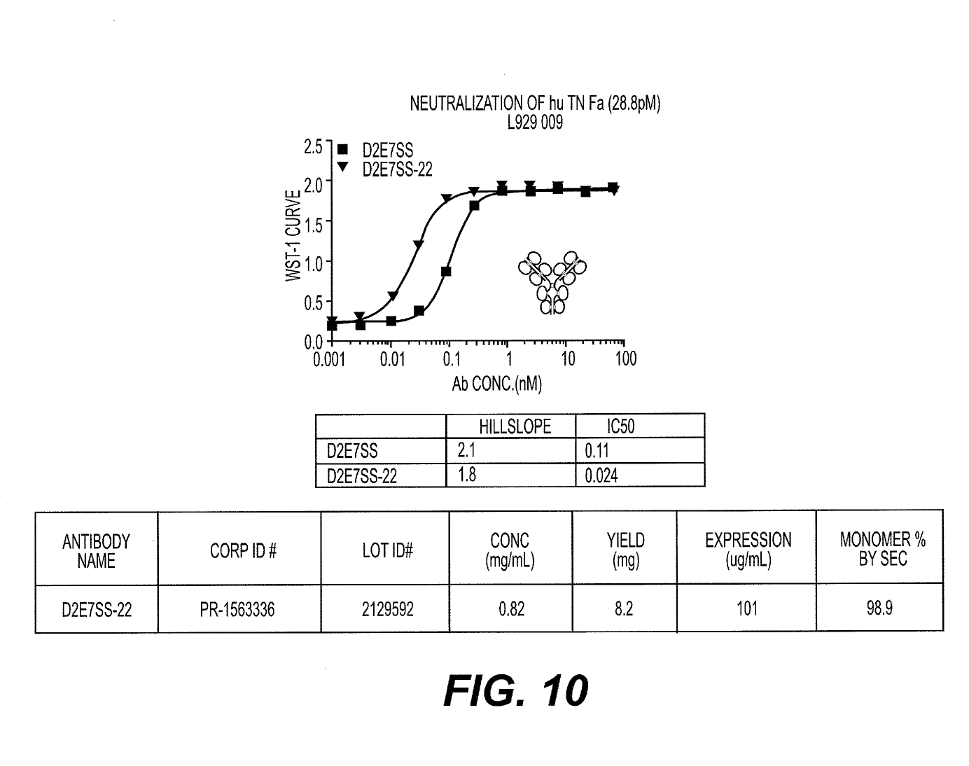

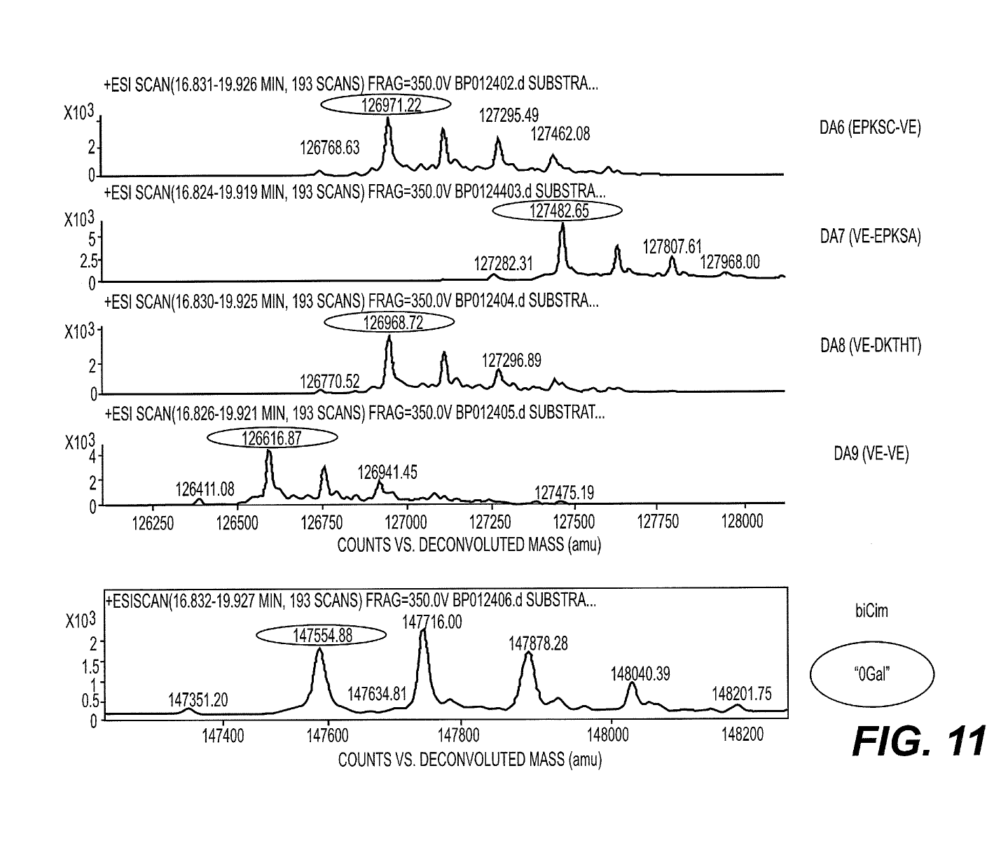

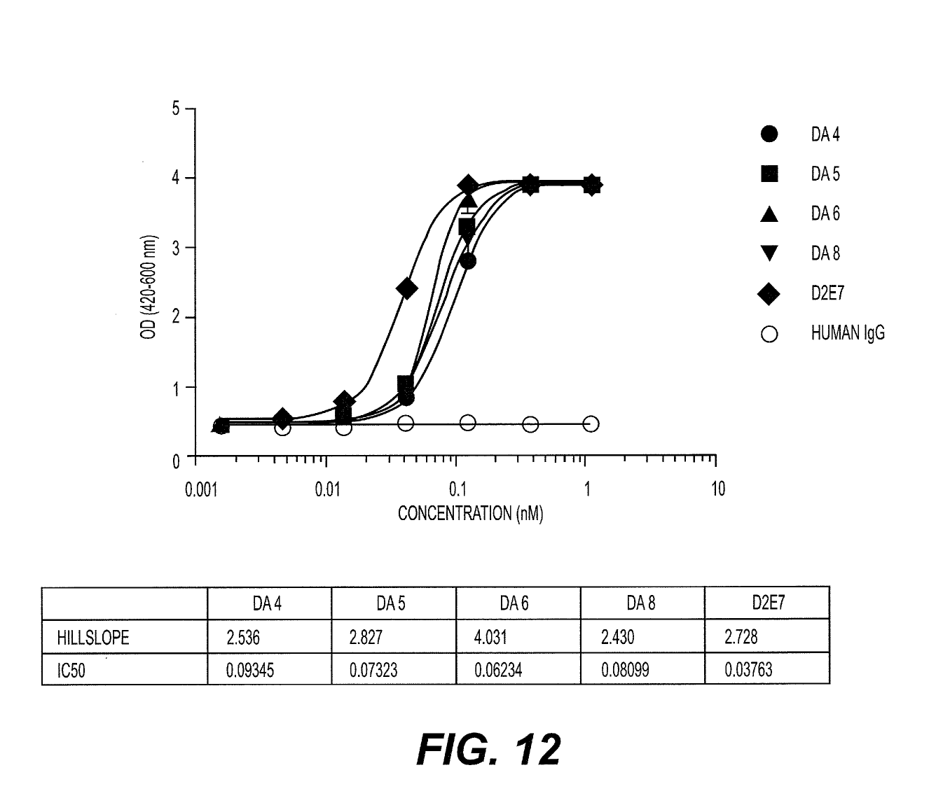

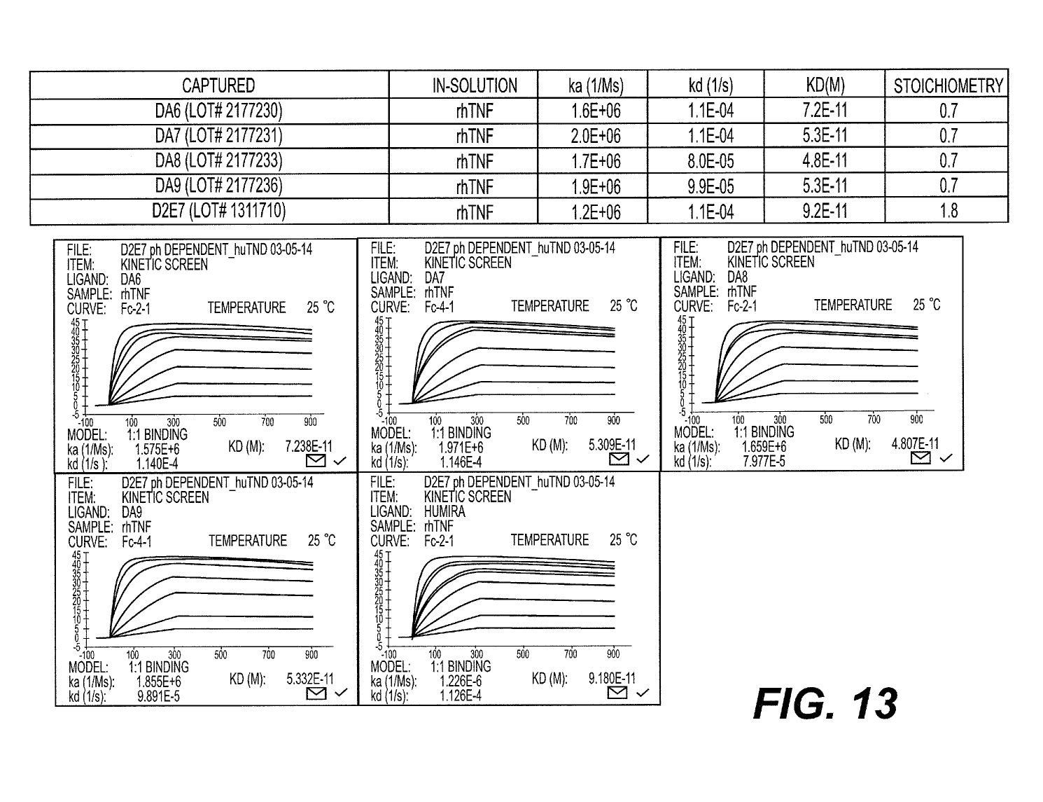

Representative results for Ambromab EPKSC-EPKSA D2E7-GS10-AB420, as compared to anti-TNFα antibodies (D2E7), are shown in FIG. 5. A comparison of EPKSC-EPKSA, EPKSC-DKTHT, VE-DKTHT and VE-VE D2E7-GS10-AB420 bispecific Ambromab molecules with respect to anti-TNFα antibodies (D2E7) is shown in FIGS. 10-11. A comparison of EPKSC-EPKSA, EPKSC-DKTHT, VE-DKTHT and VE-VE D2E7SS22-GS10-AB420 bispecific Ambromab molecules with respect to anti-TNFα antibodies (D2E7) is shown in FIG. 15.

### Example 6

### Mass Spectrometry Analysis of Ambromab Molecules

To measure the intact molecular weight of the EPKSC-EPKSA, EPKSC-DKTHT, VE-DKTHT and VE-VE D2E7-GS10-AB420 bispecific Ambromab molecules, 2 μL of an Ambromab molecule (0.8 μg/μL) was injected onto a Poroshell 300 SB-C3 column (1.0×75 mm, 5 μm, Agilent Technologies Inc., Pala Alto, Calif.). The LC/MS analysis was performed on an Agilent HP1200 Capillary HPLC connected to a mass spectrometer Agilent 6224 TOF LC/MS system (Agilent Technologies Inc., Pala Alto, Calif.). Buffer A was 0.1% formic acid in water, and buffer B was 0.1% formic acid in acetonitrile. The flow rate was 50 μL/minute. The separation gradient was held at 5% B for the first 5 minutes, increased to 95% B in 0.5 minute and was held at 95% B for the next 9.5 minutes before changed to 5% B in 0.5 minute and was held at 5% B for another 4.5 minutes. The mass spectrometer was operated at 5 (volts spray voltage and scan range was from 600 to 3200 mass to charge ratio.

To measure the molecular weight (MW) of light and heavy chains of a protein sample, 10 μl of protein sample (0.8 μg/μl) was reduced by 0.2 μL 1 M DTT solution at 37° C. for 30 minutes. A Poroshell 300SB-C3 column, 1.0×75 mm, 5 μm (Agilent Technologies Inc., Pala Alto, Calif.) was used to separate the light chain and heavy chain. The LC/MS analysis was performed on an Agilent HP1200 Capillary HPLC connected to a mass spectrometer Agilent 6224 TOF LC/MS system (Agilent Technologies Inc., Pala Alto, Calif.). Buffer A was 0.1% formic acid in water, and buffer B was 0.1% formic acid in acetonitrile. The flow rate was 50 μl/minute, and the sample injection volume was 2 μL. The column temperature was set at 60° C. The separation gradient started at 5% B. Increased to 35% in 5 minutes, then increased to 65% B in 15 minutes, increased to 95% B in 1 minute and held at 95% for 4 minutes, and decreased to 5% B in 1 minute and held at 5% B for 5 minutes. The mass spectrometer was operated at 5 kvolts spray voltage and scan range was from 600 to 3200 mass to charge ratio.

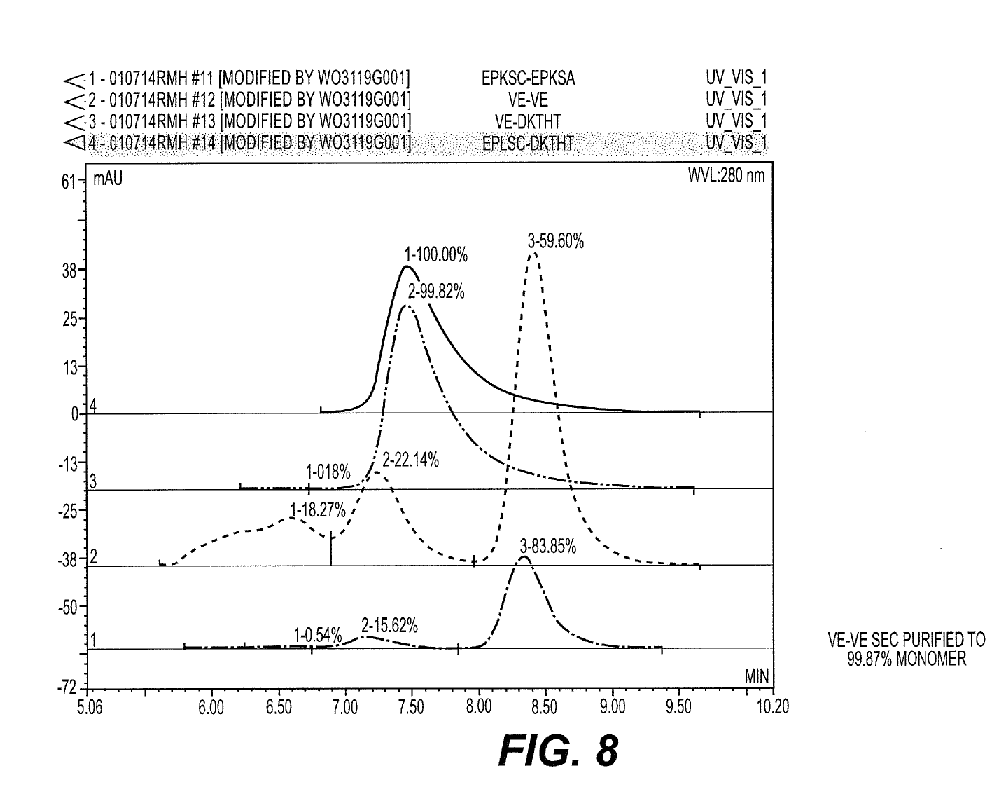

As shown in FIG. 8 and Table 10 below, the experimentally determined molecular mass of EPKSC-EPKSA, EPKSC-DKTHT, VE-DKTHT and VE-VE D2E7-GS10-AB420 bispecific Ambromab molecules, including the light chain, heavy chain, and the full-length protein, is in good agreement with the predicted value.

TABLE 10
Actual and predicted sizes of various monvalent-
bispecific D2E7-GS10-AB420 Ambromab hinge variants.

| | | VH | “VLM |
|---|---|---|---|
| | | size (1445 | size (1445 |
| | | added for sugar, | added for sugar, |
| | Predicted | C term lysine | C term lysine |
| Molecule | size KDa | removed) | removed) |
| D2E7-GS10-AB420 | 127.791 | 64776.5 | 63013 |
| EPKSC-EPKSA | | | |
| D2E7-GS10-AB420 | 127.278 | 64776.5 | 63012.9 |
| EPKSC-DKTHT | | | |
| D2E7-GS10-AB420 | 126.924 | 64422.2 | 63012.9 |
| VE-DKTHT | | | |
| D2E7-GS10-AB420 | 126.57 | 64422.2 | 62146.06 |
| VE-VE | | | |

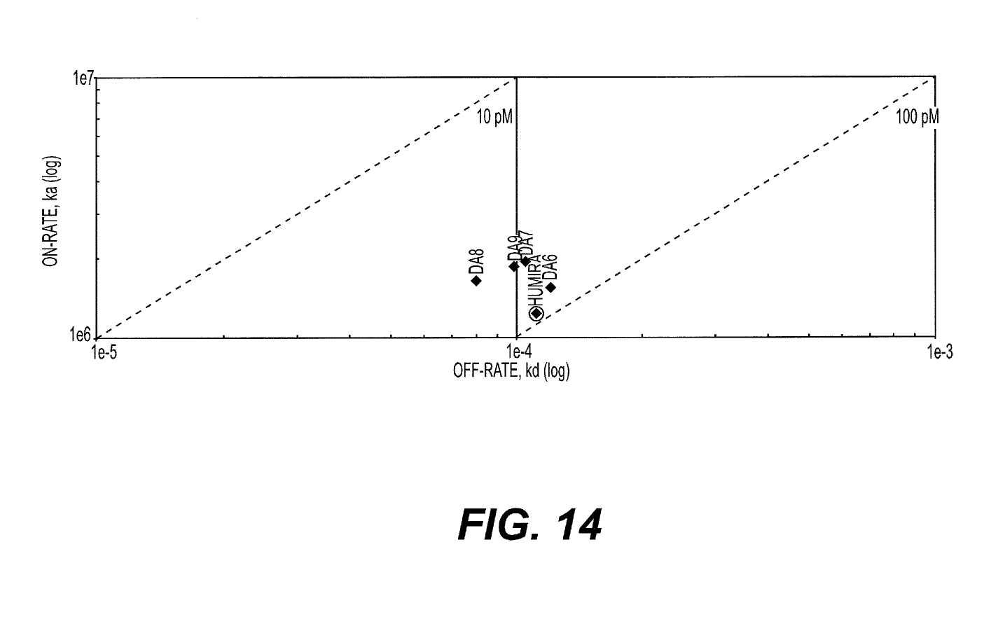

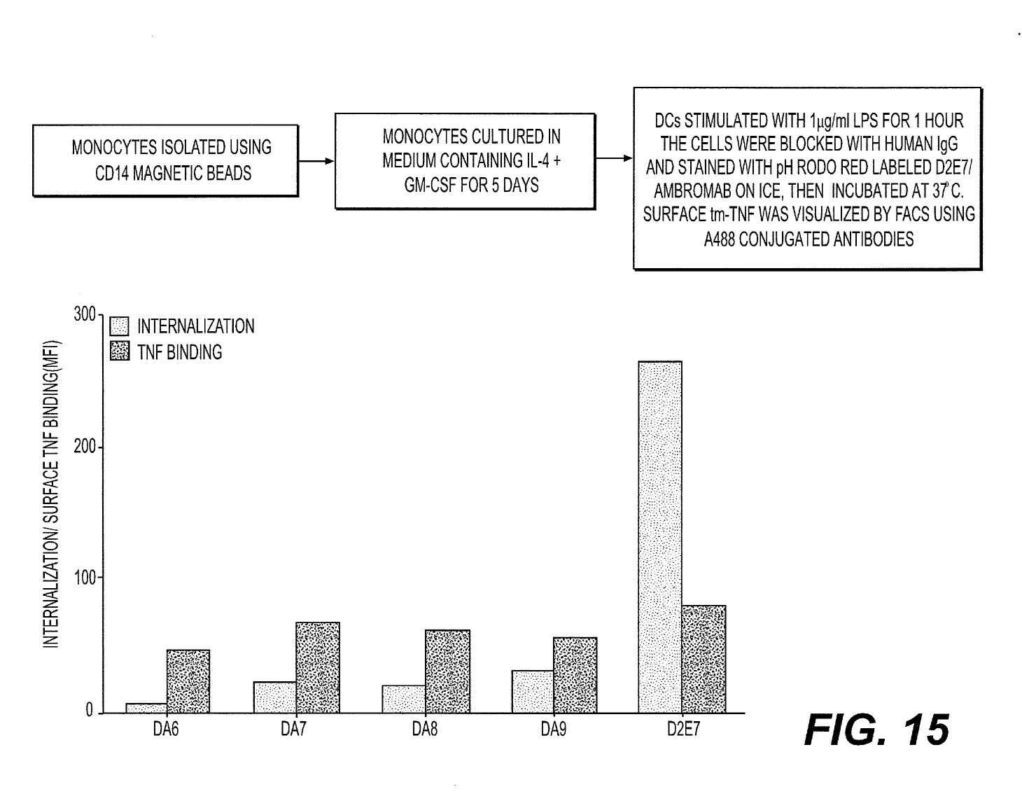

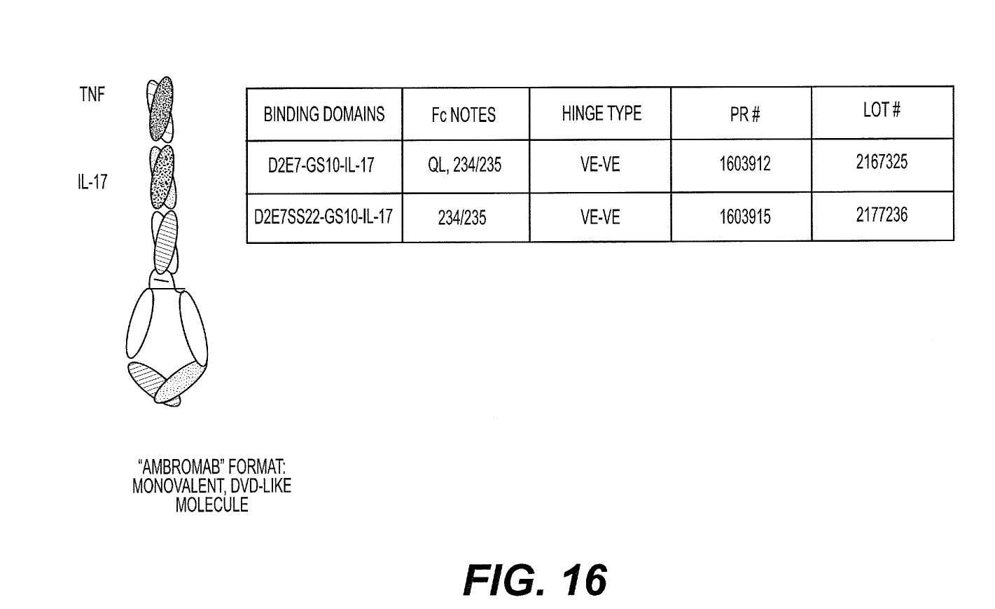

Table 11 shows the experimentally determined molecular mass of EPKSC-EPKSA, EPKSC-DKTHT, VE-DKTHT and VE-VE D2E7SS22-GS10-AB420 bispecific Ambromab molecules, including the light chain and heavy chain, and is in good agreement with the predicted value. The Non-reduced mass spec profiles are shown in FIG. 14.

TABLE 11
D2E7SS22-GS10-AB420 Ambromab hinge variants with
predicted/observed molecular weights and percent
purification after size exclusion chromatography.

| | | | | SEC |
|---|---|---|---|---|
| | | | | after 2^{nd} |
| | | Predicted | | round |
| DA# | Molecule | VH/VL | observed | purification |
| 6 | D2E7SS22-GS10- | 64789/62192 | ?/62188 | 99.8% |
| | AB420 | | | |
| | EPKSC-VE | | | |
| 7 | D2E7SS22-GS10- | 63890/62289 | 63839/62851 | 99.8% |
| | AB420 | | | |
| | VE-EPKSA | | | |
| 8 | D2E7SS22-GS10- | 63890/62546 | 63841/62542 | 99.4% |
| | AB420 | | | |
| | VE-DKTHT | | | |
| 9 | D2E7SS22-GS10- | 63890/62192 | 63839/62188 | 99.8% |
| | AB420 | | | |
| | VE-VE | | | |

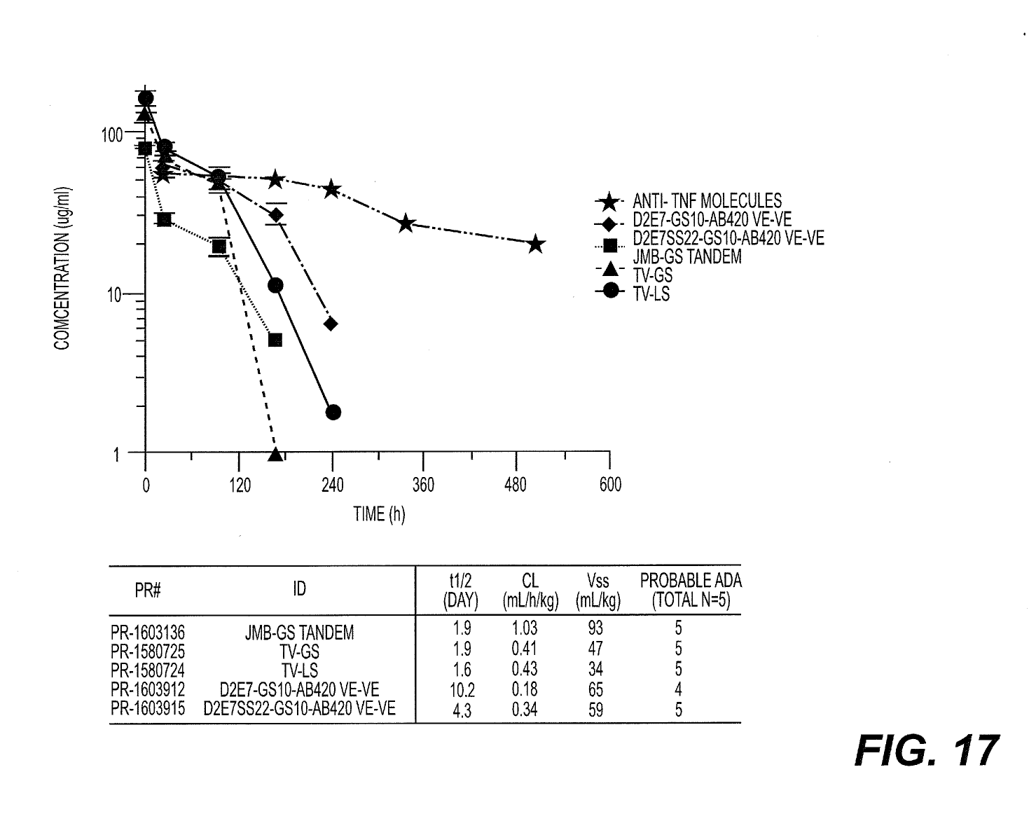

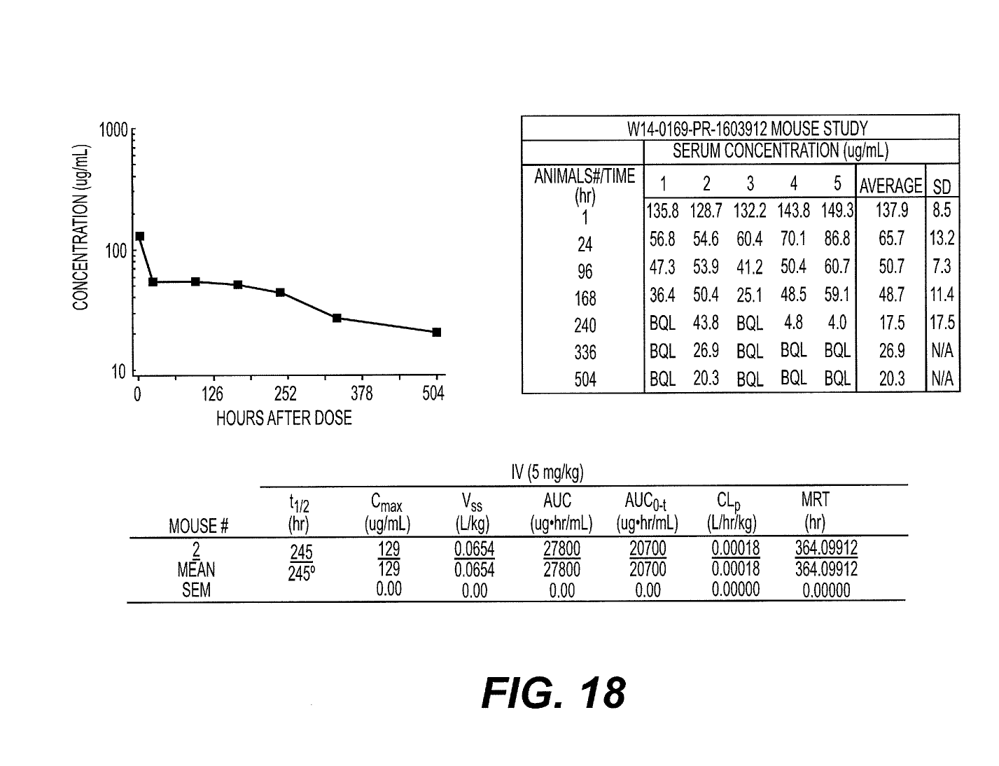

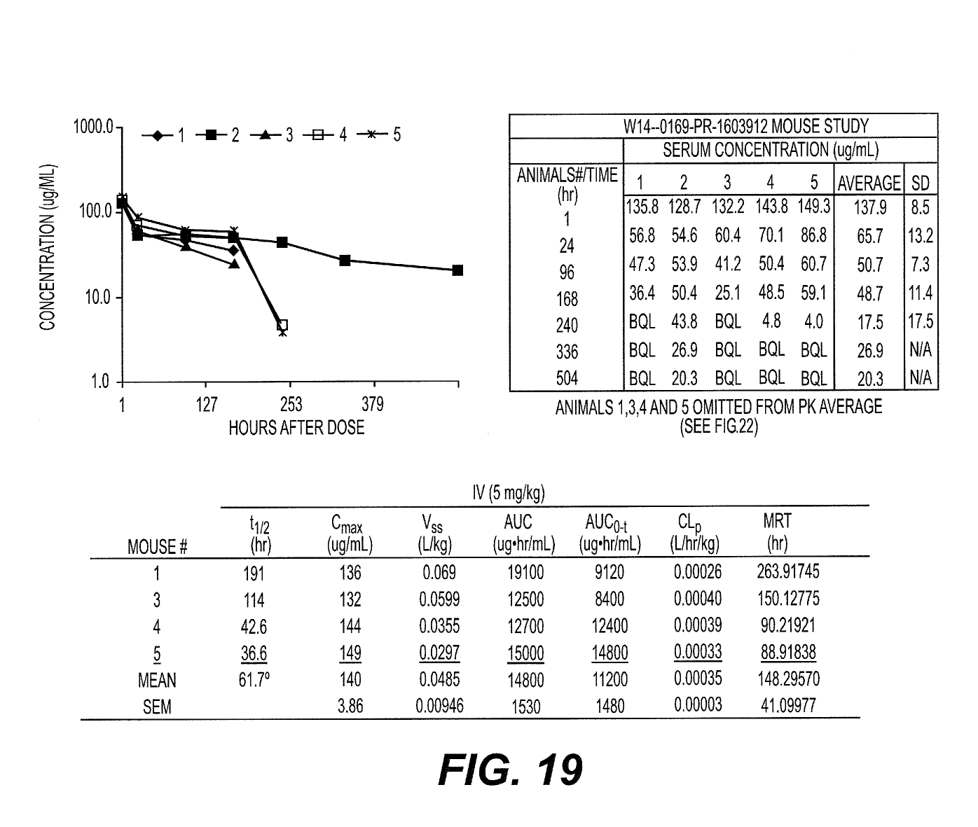

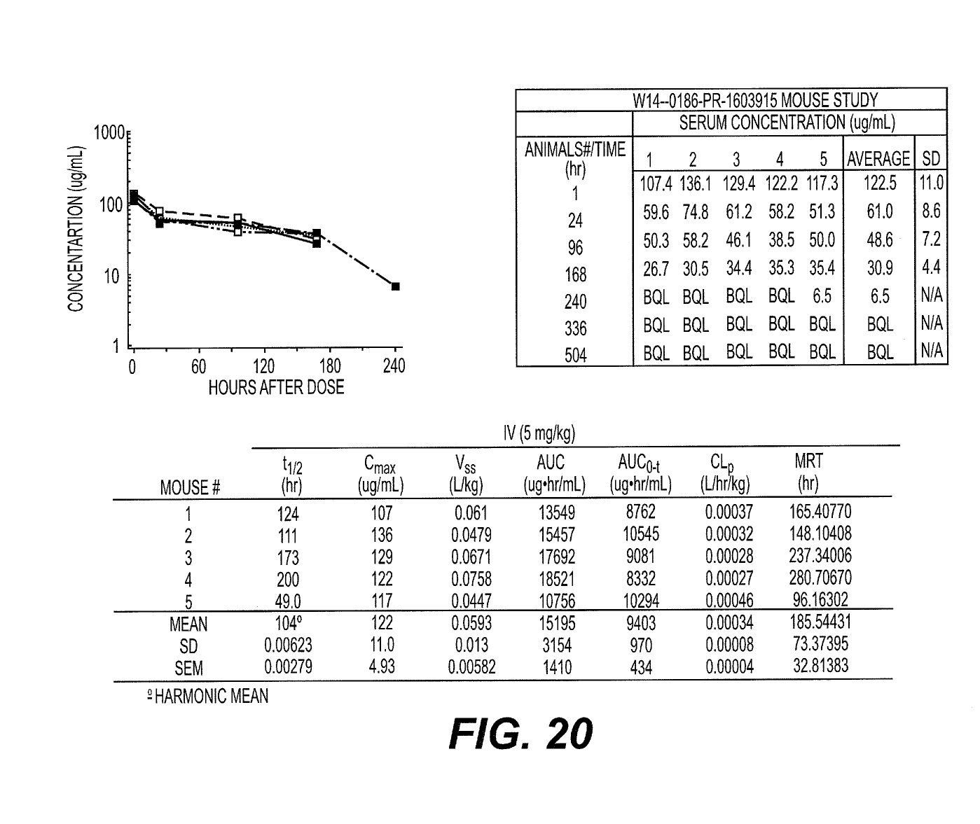

A summary of the kinetic rate parameters for EPKSC-EPKSA, EPKSC-DKTHT VE-DKTHT and VE-VE D2E7-GS10-AB420 bispecific Ambromab molecules as compared with D2E7 is shown in FIG. 17. The on-off rate map of the Ambromab molecules as compared to D2E7 is shown in FIG. 18.

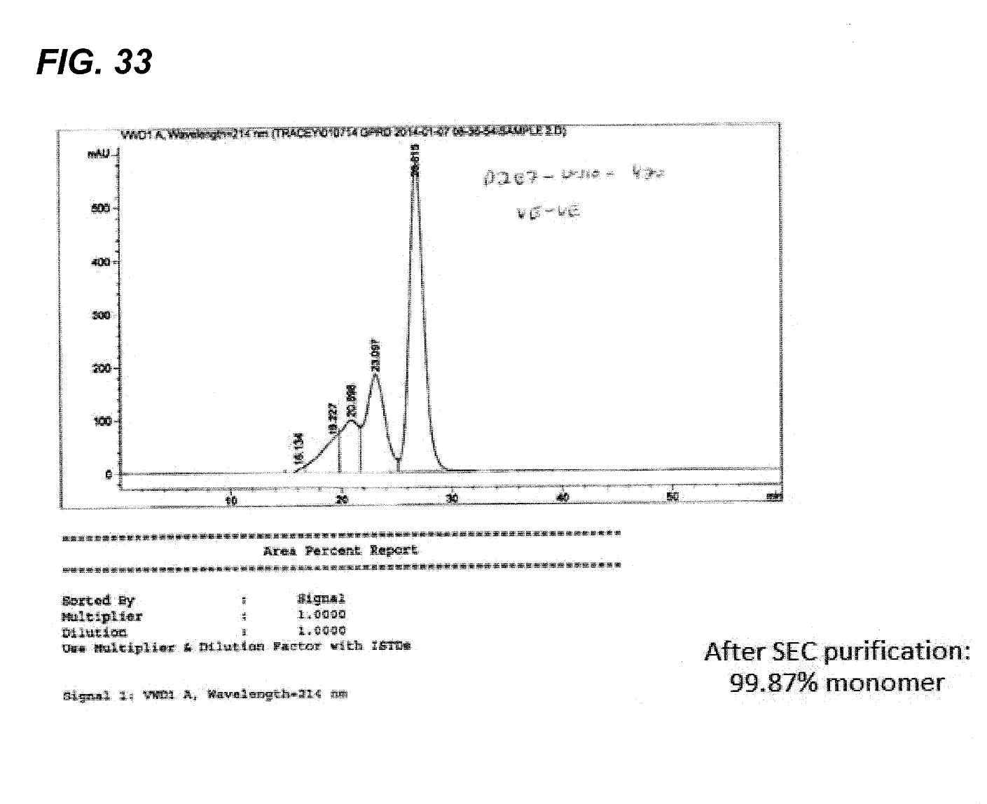

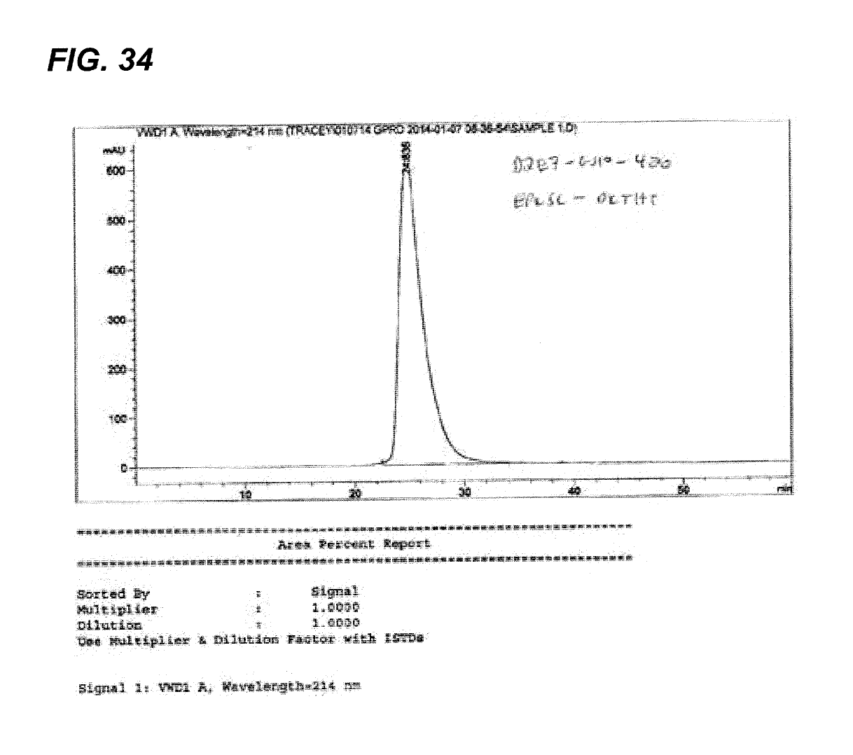

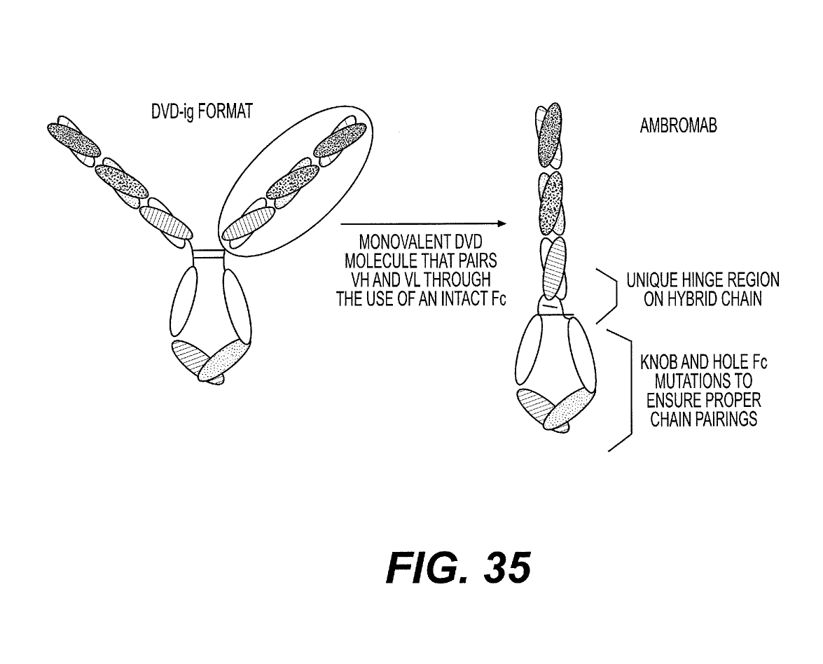

The mass spectrometry profile under reducing conditions of purified D2E7-GS10-420 EPKSC-EPKSA 234 235 QL is shown in FIG. 33. The mass spectrometry profile under reducing conditions of purified D2E7-GS10-420 VE-VE 234 235 QL is shown in FIG. 34. The mass spectrometry profile under reducing conditions of purified D2E7-GS10-420 VE-DKTHT 234 235 QL is shown in FIG. 35. The mass spectrometry profile under reducing conditions of purified D2E7-GS10-420 EPKSC-DKTHT 234 235 QL is shown in FIG. 36.

### Example 7

### TNFα Binding and Internalization Ambromab Variants

### Isolation of Monocytes, Culture and Stimulation

Peripheral blood mononuclear cells (PBMC) were isolated from leukopack of healthy donors by density gradient centrifugation over Ficoll-Paque (GE Health Sciences). Monocytes were isolated by magnetic sorting using CD14 microbeads (Mitenyi Biotec). The purity of the resulting monocytes, as assessed by flow cytometric analysis, was typically greater than 98%. Monocytes were cultured in RPMI1640 medium (Cellgro) supplemented 2 mM L-glutamine, 100 ng/ml of recombinant human GM-CSF (AbbVie) and 5 ng/ml of human IL-4 (Peprotech), 100 μg/ml penicillin, and streptomycin, and 10% fetal bovine serum at a density of 1×10⁶ cells/ml at 37° C. with 5% CO₂ for 5 days.

To test the surface TNFα expression, PBMCs or monocytes were stimulated with ultra-low (0.025 ng/ml), low (0.25 ng/ml) or high (250 ng/ml) of LPS (from Salmonella typhimurium, Sigma-Aldrich) for indicated period.

### Dendritic Cell Differentiation and Stimulation

Dendritic cells were generated by culturing monocytes in RPMI1640 medium supplemented with 100 ng/ml of recombinant human GM-CSF (AbbVie) and 5 ng/ml of human IL-4 (Peprotech) for 4 days. To investigate the TNFα production DCs were stimulated with 1 mg/ml LPS (from Salmonella typhimurium, Sigma-Aldrich) for 1 hour.

### Staining Cells and Flow Cytometric Analysis

LPS stimulated PBCs, monocyte or DCs were blocked with human IgG and stained with pHrodo red labeled D2E7/Ambromab on ice, then incubated at 37° C. As a negative control an isotype matched control antibody (AB446) was used. All the antibodies were conjugated with A488 using antibody labeling kit (Invitrogen) according to the manufacturer's protocol. Monocytes and T cells were gated based on the expression of CD14 (Biolegend) and CD3 (eBioscience) respectively. Samples were analyzed on a Becton Dickinson Fortessa flow cytometer, and analysis was performed using Flowjo software (TreeStar Inc., Ashland, Oreg., USA).

### Internalization Assay

To investigate the internalization of surface TNF bound Ambromab antibodies, monocytes were stimulated with LPS for 4, 7, 9 or 24 hours in the presence of Alexa 488 conjugated AB436 antibodies. Cells were permeabilized and nucleus was stained with DAPI. The images were acquired using confocal microscope (Zeiss). To study the internalization of anti-TNF Ambromab antibodies by dendritic cells, the monocyte derived DCs were stimulated with LPS for 4 hours in the presence of anti-TNF Ambromab or matched isotype control antibodies. The Anti-TNFα specific Ambromab antibodies and control antibodies were conjugated with pH sensitive dye pHRodo Red (Invitrogen) according to manufacturer's protocol. The cells were analyzed by fluorescent microscope and FACS. Where indicated, the surface of the cells was stained with A488-conjugated anti-HLA-A.B.C. (W6/32, Biolegend) antibodies and the nucleus was stained with Nuce blue (Invitrogen). To study the internalization kinetics of anti-TNFα Ambromab antibodies by membrane TNF on DCs, cells were either left in un-stimulated or stimulated with LPS for 1 hour or 24 hours. The surface TNFα was stained with pHRodo Red conjugated anti-TNFα antibody (AB441). The stained cells were cultured in RPMI medium for indicated time and the internalization was assessed as increase in fluorescence using BD Fortessa flow cytometer.

### Cell Surface Biotinylation

Cells (2-3×10⁶) were washed twice with ice-cold PBS-CM (PBS containing 1 mM CaCl₂ and 1 mM MgCl₂) and the cell surface proteins derivatized twice by using 1 mg/ml cell-impermeable EZ-Link-Sulfo-NHS-SS-Biotin in PBS-CM on ice for 30 minutes protected from light with gentle agitation. Excess biotin was quenched by incubating the cells for 10 minutes on ice in 50 mM NH4Cl. Cells were washed twice with PBS-CM and total proteins extracted in 150 μl lysis buffer (50 mM Tris-HCl, pH 7.4, 150 mM NaCl, 50 mM n-Octyl-β-D-glucoside, 0.5% sodium deoxycholate, 1 tablet EDTA-free protease inhibitor cocktail in 7 ml lysis buffer, 1 mM PMSF) on ice for 45 minutes and centrifuged at 12,000× g for 10 minutes at 4° C. Clarified supernatant was transferred to a fresh microcentrifuge tube on ice and total proteins estimated by the BCA (Bicinchoninic acid) protein assay reagent. To enrich the cell surface biotinylated proteins, 25-75 μg total proteins were transferred to fresh tube and the volume made to 500 μl using the lysis buffer and mixed with 50 μl streptavidin-conjugated agarose beads. The tube was mixed end-over-end at 4° C. overnight. The agarose beads were collected by centrifugation at 2,500×g for 3 minutes and sequentially washed twice by suspending in 1 ml fresh ice-cold lysis buffer, once in 1 ml ice-cold 500 mM NaCl, and once in 1 ml 50 mM Tris-HCl, pH 8. The streptavidin-agarose bound, cell surface biotinylated proteins, along with 6-15 μg total proteins in a separate tube, were suspended in 40 μl SDS-PAGE sample buffer containing 4M urea and 5% β-mercaptoethanol, separated on 4-20% Novex Tris-Glycine SDS-PAGE, and transferred onto a 0.2 μm nitrocellulose membrane for 1 hour. The nitrocellulose membrane was incubated in 5% non-fat dry milk in TBS-T (25 mM Tris-HCl, 150 mM NaCl, pH 7.5, containing 0.2% Tween-20) for 30 minutes at room temperature with gentle agitation, washed once in TBS-T for 5 min at room temperature and incubated overnight with gentle agitation at 4° C. in the following primary antibodies: (1) Rabbit-Pan Cadherin IgG (1:1000 in 5% bovine serum albumin, BSA, in TBS-T); (2) FITC Mouse anti-Human CD14 IgG (1:500 in 5% non-fat dry milk, in TBS-T); (3) Human anti-Human TNF-alpha D2E7-GS10-AB420 VE-VE Ambromab (1:1000 in 5% non-lat dry milk, in TBS-T); and (4 Rabbit anti-GAPDH IgG (1:5000 in 5% non-fat dry milk in TBS T).

The next day, the membrane was washed twice for 15 minutes each with TBS-T with vigorous agitation at room temperature. The membrane was incubated in the appropriate horseradish peroxidase (HRP)-conjugated secondary IgG in 5% non-fat dry milk in TBS-T for 45 at room temperature with gentle agitation and washed twice for 15 min each in TBS-T with vigorous agitation at room temperature. The membrane was incubated either in ECL or ECL Prime western blotting analysis systems and exposed to X-ray films for various periods of time.

### Example 8

### Pharmacokinetic Studies

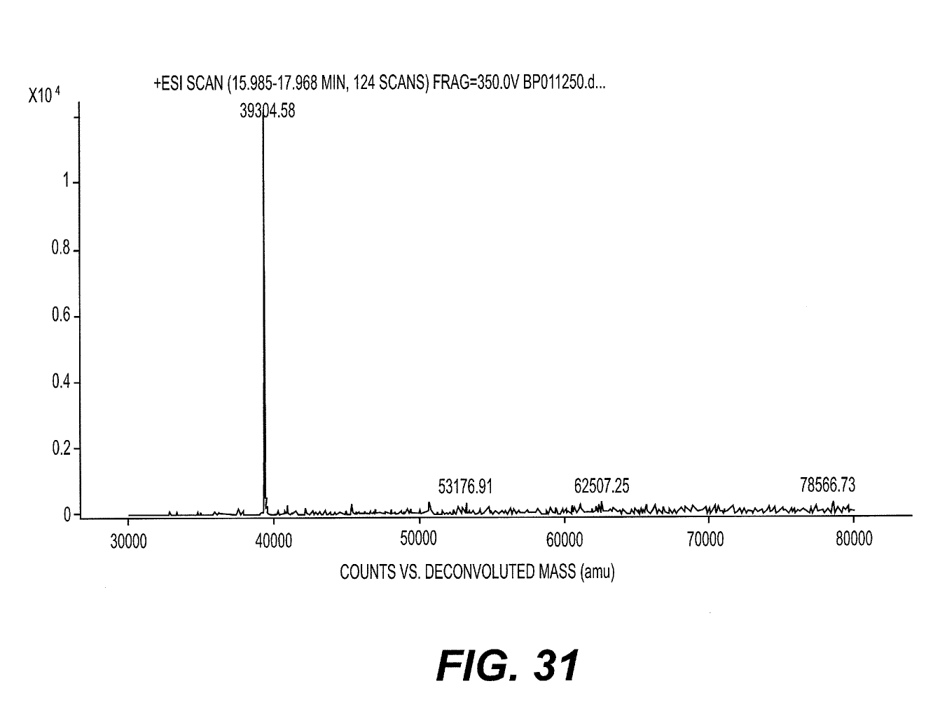

D2E7-GS10-AB420 VE-VE and D2E7SS22-GS10-AB420 VE-VE Ambromab molecules were administered to CD-1 mice by slow intravenous bolus dose injection at a 5 mg/kg dose. Blood samples were collected from each mouse at 1, 24 and 96 hours and 7, 10, 14 and 21 days post dose. Blood samples were collected from each rat at 0.25, 4, and 24 hours and 2, 3, 7, 10, 14, 21 and 28 days post dose. All samples were stored at −80° C. until analysis. Serum samples were analyzed an anti-TNF capture assay depicted in FIG. 31 in which a biotinylated human TNFα is used for capture and a labeled anti-human Sulfo-Tag h for detection. The assay was carried out in 1% final serum concentration. The lower limit of quantitation (LLOQ) was 0.004 μg/mL. The linear range: 15-0.004 μg/mL. The low control was 0.1 μg/mL.

Standard curve fitting and data evaluation was performed using XLfit4 software with a four-parameter logistic fit. Plates passed when at least 2/3 of the QC's were within 30% of the expected values. Pharmacokinetic parameters for each animal were calculated using WinNonlin software Version 5.0.1 (Pharsight Corporation, Mountain View, Calif.) by non-compartmental analysis using linear trapezoidal fit (NCA Models #201 for IV dosing). For calculations in WinNonlin, the time of dosing was defined as Day 0 Time 0 hour.

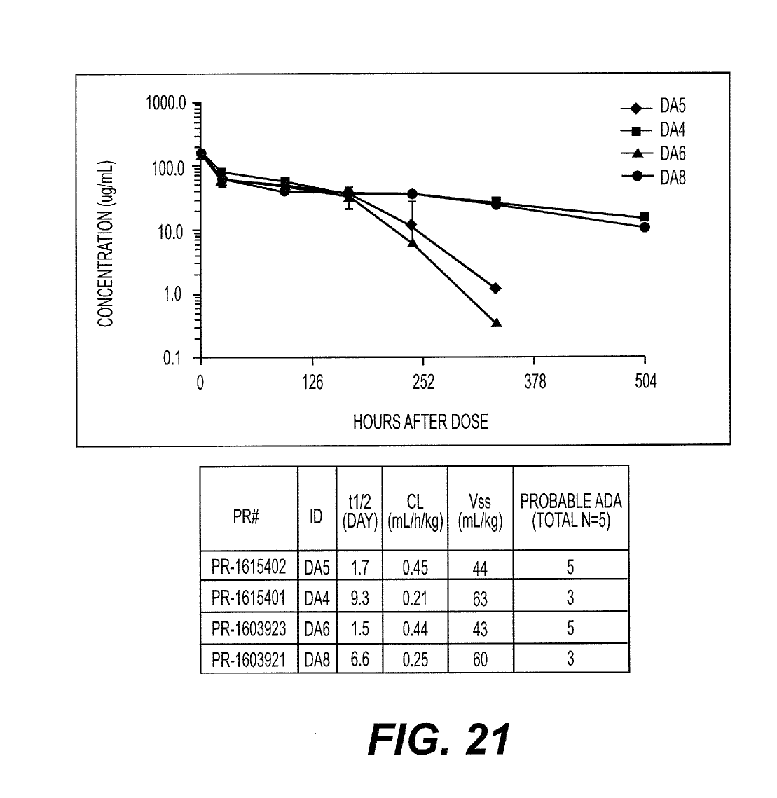

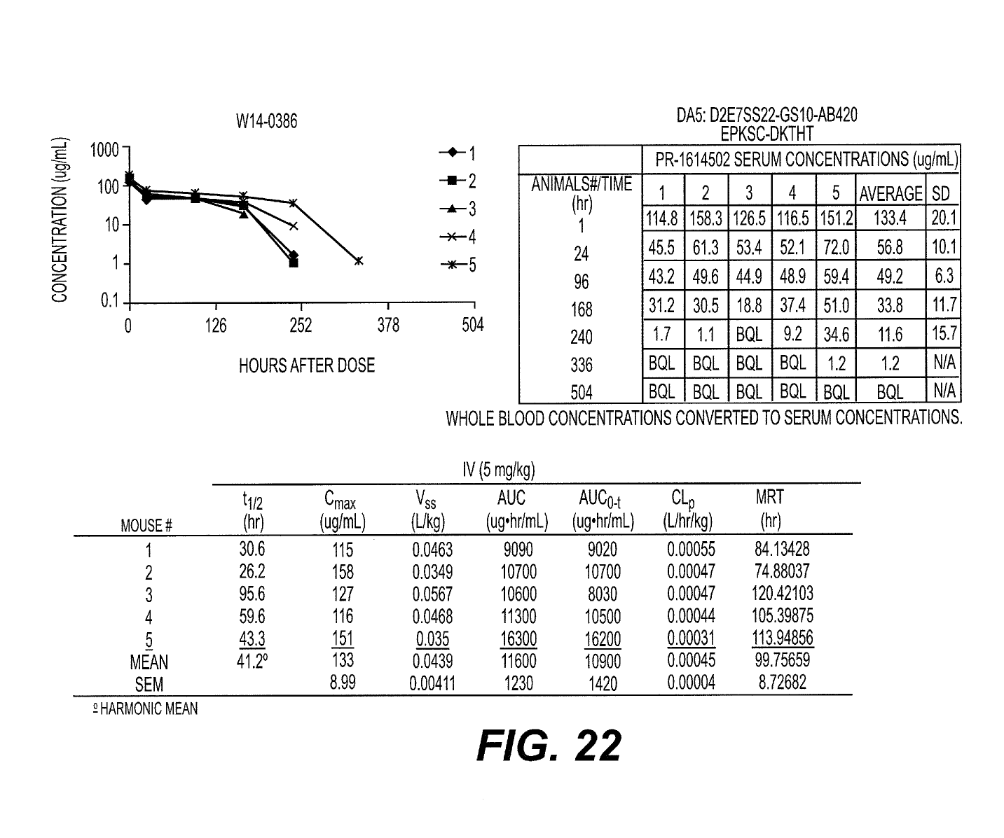

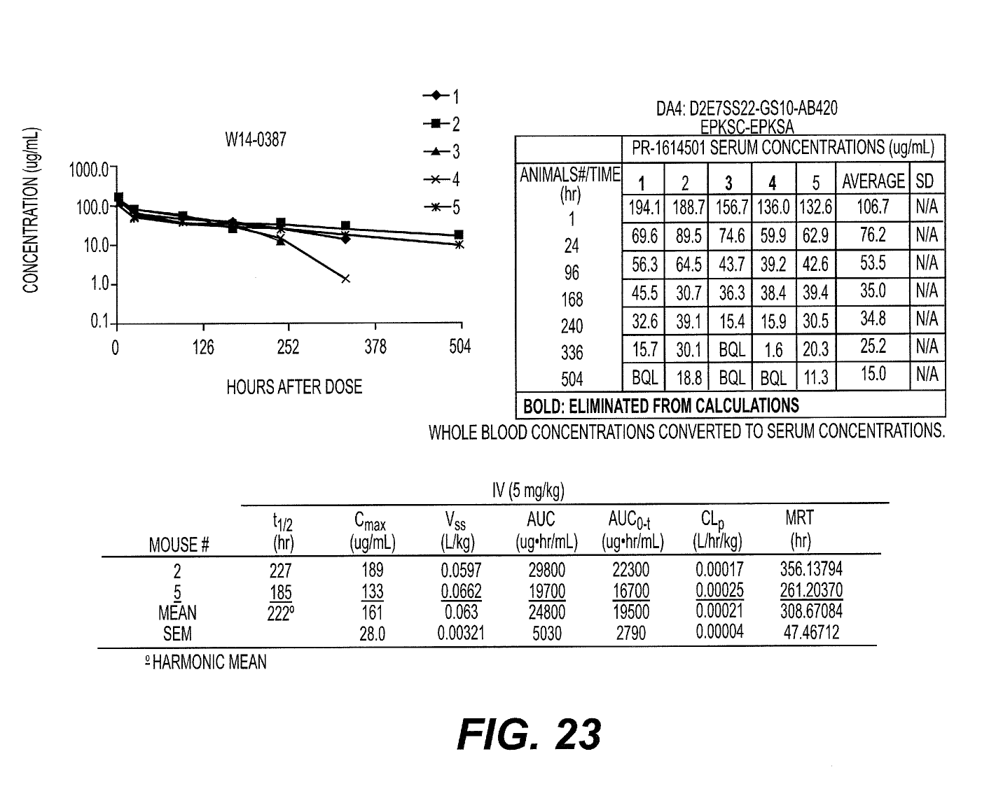

The results are shown in FIG. 21, 1 of 5 animals administered anti-TNFα monovalent molecule D2E7-GS10-AB420 VE-VE (PR-1603912) had measurable antibody levels out to 21 days. This animal displayed a long half-life and low CL (10.2 days and 0.18 mL/h/kg; see FIG. 22). All other animals within this group and all other dose groups displayed probable anti-drug antibodies (ADA) (see FIG. 23). The short half-lives for PR-1603136, PR-1580725, PR-1580724, and PR-1603915 prior to the onset of ADA could be due to a number or mechanisms including renal clearance and in vivo stability.

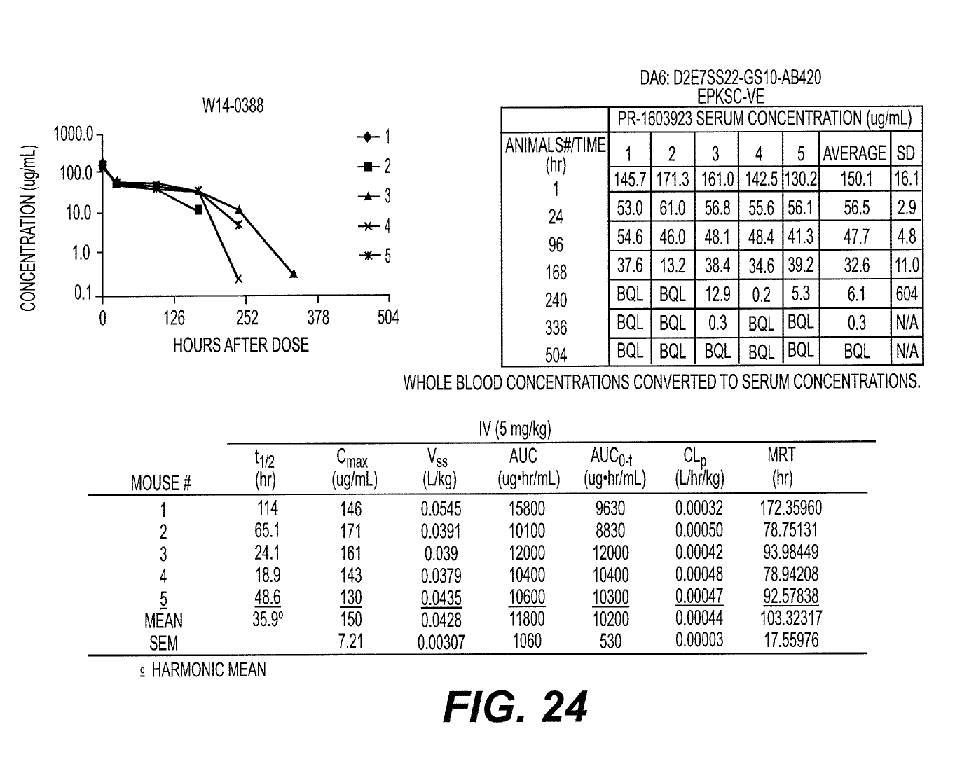

The serum concentrations of anti-TNFα D2E7SS22-GS10-AB420 VE-VE Molecule (PR-1603915) in 5 different animals is shown in FIG. 24.

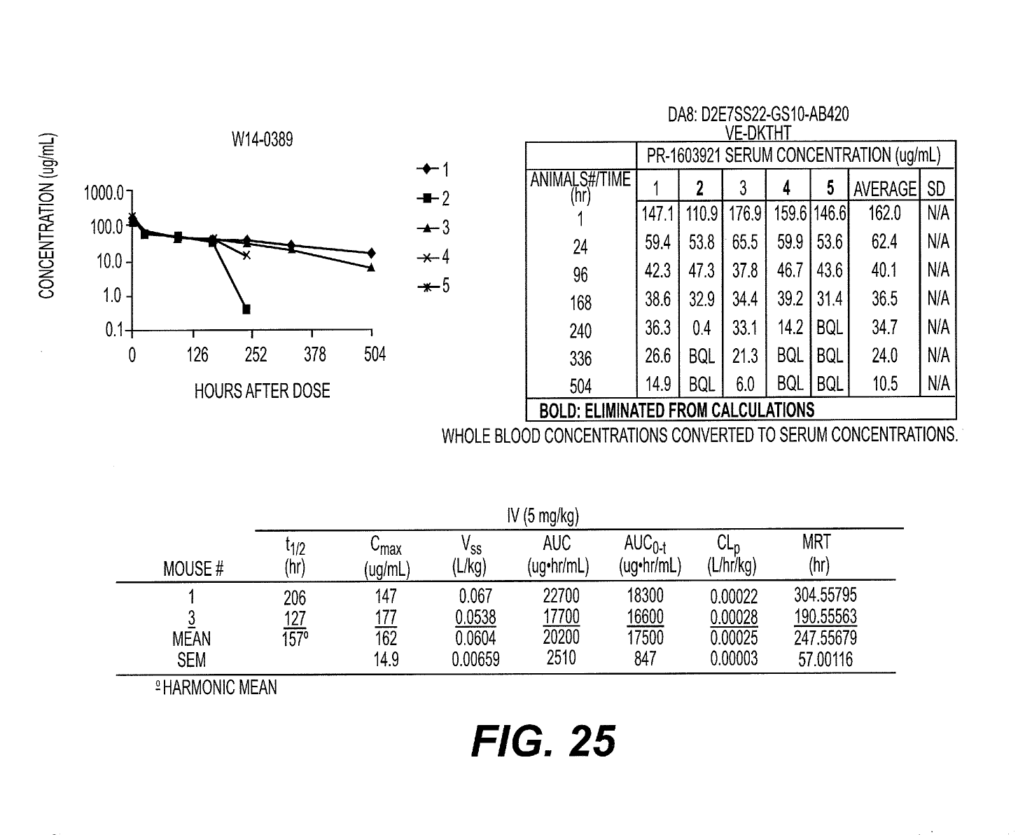

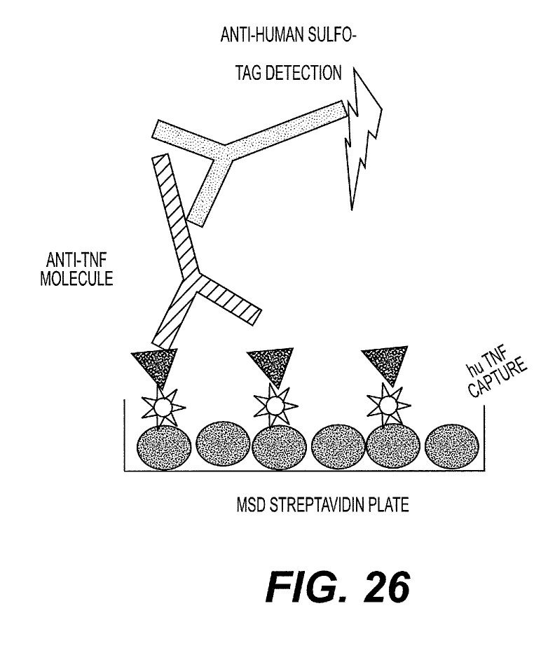

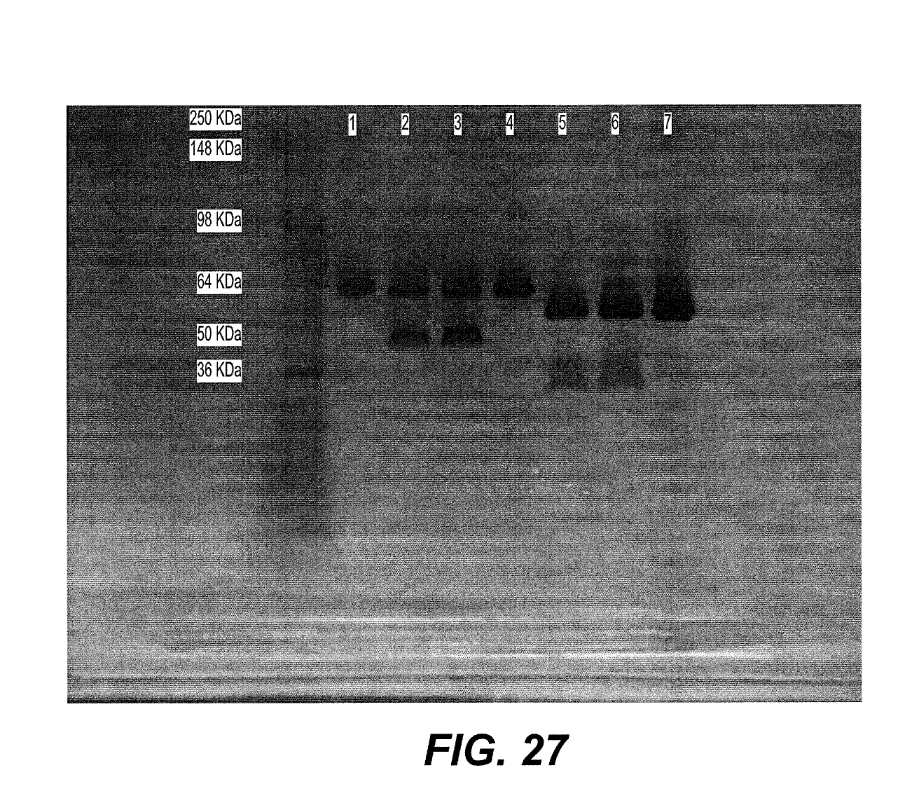

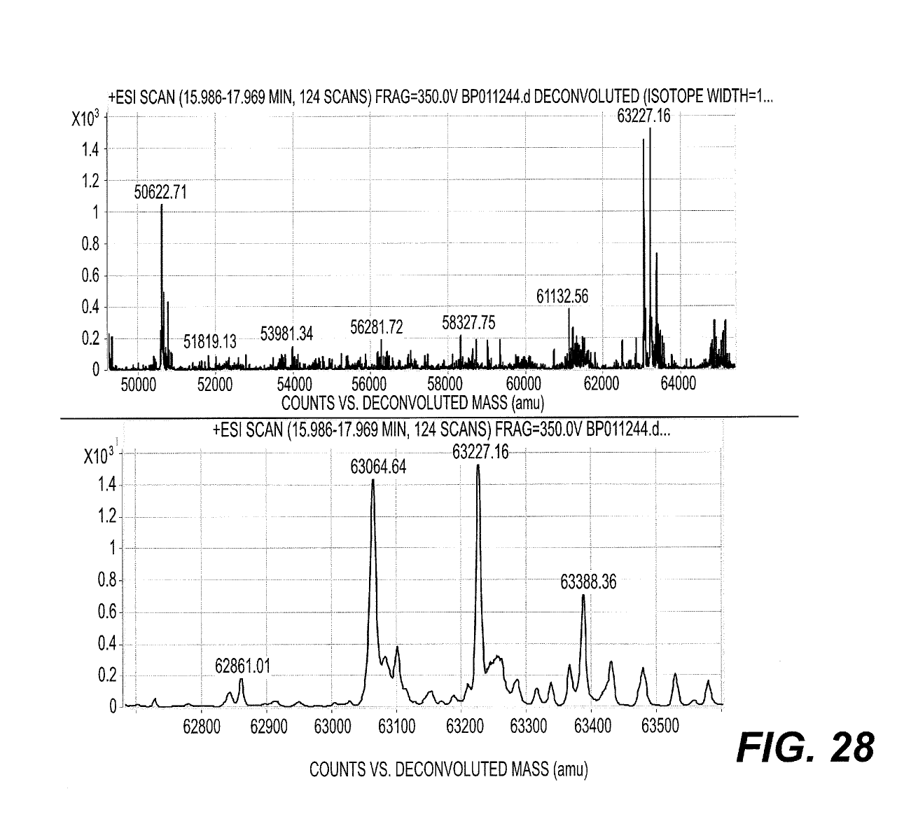

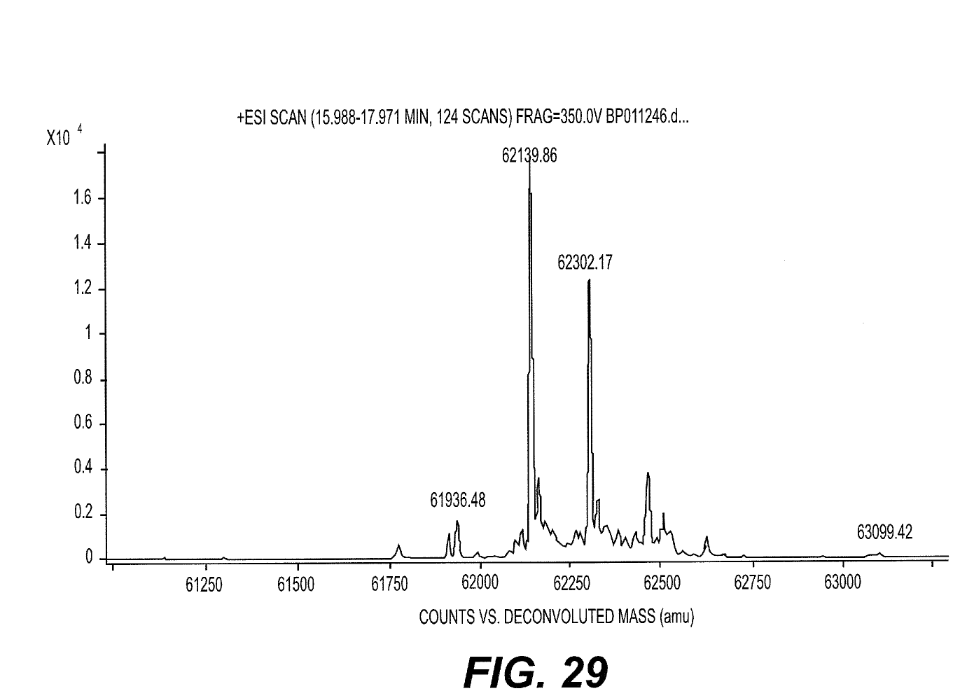

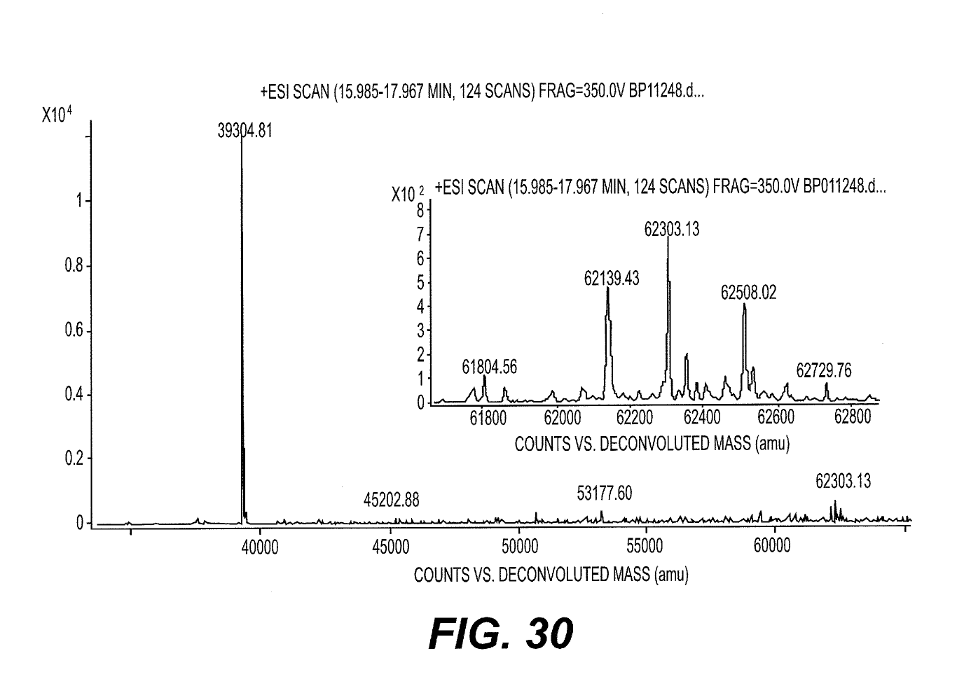

A summary of the pharmacokinetics of anti-TNFα DVD-like Ambromab molecules DA4, DA5, DA6 and DA8 after 5 mg/kg IV dosing in CD-1 mice is shown in FIG. 25. Ambromab molecules DA4 and DA8 displayed similar PK parameters, with moderate half-lives (˜9 and 7 days), low CL (0.21 and 0.25 mL/h/kg), and small Vss (63 and 60 mL/kg) for DA4 and DA8 respectively. Probable ADA was seen in 3 animals in each dose group. DA5 and DA6 showed measurable concentrations out to 14 days due to probable ADA. Both had short t_{1/2}(˜2 days), moderate CL (˜0.45 mL/h/kg), and small Vss (44 mL/kg). The pharmacokinetics of anti-TNFα Ambromab molecule DA5 (PR-1614502) serum concentrations in 5 CD-1 mice after 5 mg/kg IV dosing (W14-0386) is shown in FIG. 26. The pharmacokinetics of anti-TNFα Ambromab molecule DA4 (PR-1614502) serum concentrations in 5 CD-1 mice after 5 mg/kg IV dosing (W14-0386) is shown in FIG. 27. The pharmacokinetics of anti-TNFα Ambromab molecule DA6 (PR-1614502) serum concentrations in 5 CD-1 mice after 5 mg/kg IV dosing (W14-0386) is shown in FIG. 28. The pharmacokinetics of anti-TNFα Ambromab molecule DA8 (PR-1614502) serum concentrations in 5 CD-1 mice after 5 mg/kg IV dosing (W14-0386) is shown in FIG. 29. Table 12 shows a characterization of pH sensitive DVD-like Ambromab (D2E7SS22-GS10-IL-17) molecules having different hinge sequences.

TABLE 12
Characterization of pH sensitive DVD-like Ambromab (D2E7SS22-
GS10-IL-17) molecules having different hinge sequences

| | Hinge type | | | | | |
|---|---|---|---|---|---|---|
| | EPKSC- | EPKSC- | EPKSC- | VE- | VE- | |
| | EPKSA | DKTHT | VE | EPKSA | DKTHT | VE-VE |
| Total mgs from | Pending | Pending | 10.2 mgs | 9.5 mgs | 11.1 mgs | 9 mgs |
| 500 ml 293 | | | | | | |
| % monomer after | | | 99; 2.6 mgs | 99; 2.5 mgs | 99; 2.3 mgs | 99; 2.5 mgs |
| SEC; Yield impact | | | | | | |
| Yield impact | | | 75% loss | 73% loss | 79% loss | 72% |
| | | | | | | loss |
| Unexpected | Yes, faint | Yes, faint | No | No | No | No |
| smaller bands in | | | | | | |
| non-reducing SDS? | | | | | | |
| Unexpected | Yes | Yes | Yes, faint | Yes, faint | Yes, faint | Yes, |
| smaller bands in | | | | | | faint |
| reducing SDS? | | | | | | |
| L929 results | | | Yes | Yes | Yes | Yes |
| Non-red. MS | | | Correct | Correct | Correct | Correct |
| | | | size | size | size | size |
| Internationlization | | | Good | Good | Good | Good |

We claim:

1. A binding protein comprising a first and a second polypeptide chain,
wherein the first polypeptide chain comprises VH1-L1-VH2-CH1-X1-CH2-CH3, wherein
VH1 is a first heavy chain variable domain,
L1 is a linker,
VH2 is a second heavy chain variable domain,
CH1, CH2 and CH3 are heavy chain constant domains 1, 2 and 3, respectively
X1 comprises a first immunoglobulin hinge region; and
wherein the second polypeptide chain comprises VL1-L2-VL2-CL-X2-CH2-CH3, wherein
VL1 is a first light chain variable region,
L2 is a linker,
VL2 is a second light chain variable region,
CL is a light chain constant domain,
CH2 and CH3 are heavy chain constant domains 2 and 3, respectively; and
X2 comprises a second immunoglobulin hinge region;
wherein
the first and second polypeptide chains comprise a hetero-dimerization motif that facilitates the dimerization of the first and second polypeptide chains,
wherein
VH1 and VL1 form one functional binding site for antigen A, and
VH2 and VL2 form one functional binding site for antigen B.

2. The binding protein of claim 1, wherein the hetero-dimerization motif is located in the CH3 domain of the first and second polypeptide chains.

3. The binding protein of claim 1 or 2, wherein the hetero-dimerization motif comprises knobs-into-holes mutations in the CH3 domains of the first and second polypeptide chains.

4. The binding protein of any one of claims 1-3, wherein the second immunoglobulin hinge region comprises an amino acid deletion, insertion, or substitution.

5. The binding protein of any one of claims 1-4, wherein the second immunoglobulin hinge region comprises an altered cysteine residue.

6. The binding protein of claim 5, wherein the altered cysteine residue enhances the hetero-dimerization of the first and second polypeptide chains as compared to the hetero-dimerization of first and second polypeptide chains comprising an unmodified second immunoglobulin hinge region.

7. The binding protein of claim 5, wherein the altered cysteine residue is the N terminal cysteine of the second immunoglobulin hinge region.

8. The binding protein of claim 1, wherein at least one of the first or the second immunoglobulin hinge regions comprises at least 4 continuous amino acids from the amino acid sequence EPKSCDKTHTCPPC.

9. The binding protein of claim 1, wherein the first immunoglobulin hinge region comprises the amino acid sequence EPKSCDKTHT.

10. The binding protein of claim 1, wherein the second immunoglobulin hinge region comprises the amino acid sequence EPKSXDKTHT, wherein X denotes an altered cysteine.

11. The binding protein of claim 1, wherein the second immunoglobulin hinge region comprises the amino acid sequence EPKSXDKTHT, wherein X is any amino acid except cysteine.

12. The binding protein of claim 1, wherein the second immunoglobulin hinge region comprises the amino acid sequence EPKSXDKTHT, wherein X is alanine.

13. The binding protein of claim 12, wherein the DKTHT sequence of the EPKSXDKTHT amino acid sequence within the first immunoglobulin hinge region is replaced with the amino acid sequence VE.

14. The binding protein of claim 12, wherein the EPKSXDKTHT amino acid sequence within the second immunoglobulin hinge region is replaced with the amino acid sequence VE.

15. The binding protein of claim 13, wherein the EPKSXDKTHT amino acid sequence within the second immunoglobulin hinge region is replaced with the amino acid sequence VE.

16. The binding protein of claim 12, wherein the EPKSX sequence of the EPKSXDKTHT amino acid sequence within the second immunoglobulin hinge region is deleted.

17. The binding protein of claim 13, wherein the EPKSX sequence of the EPKSXDKTHT amino acid sequence within the second immunoglobulin hinge region is deleted.

18. The binding protein of any one of claims 1-17, wherein the first and second immunoglobulin hinge regions are IgG1 hinge regions.

19. The binding protein of any one of claims 1-18, wherein the light chain constant domain is a C_{κ} (kappa) constant domain.

20. The binding protein of any one of claims 1-19, wherein the first and second polypeptide chains are covalently linked.

21. The binding protein of any one of claims 1-20, wherein antigens A and B are the same antigen.

22. A binding protein comprising a first and a second polypeptide chain,
wherein the first polypeptide chain comprises VH1-CH1-X1-CH2-CH3,
wherein
VH1 is a first heavy chain variable domain,
CH1, CH2 and CH3 are heavy chain constant domains 1, 2 and 3, respectively, and
X1 comprises a first immunoglobulin hinge region; and
wherein the second polypeptide chain comprises VL1-CL-X2-CH2-CH3,
wherein
VL1 is a first light chain variable region,
CL is a light chain constant domain,
X2 comprises a second immunoglobulin hinge region, and
CH2 and CH3 are heavy chain constant domains 2 and 3, respectively;
wherein
the first and second polypeptide chains comprise a hetero-dimerization motif that facilitates the dimerization of the first and second polypeptide chains, and
wherein
VH1 and VL1 form a functional binding site for an antigen.

23. The binding protein of claim 22, wherein the hetero-dimerization motif is located in the CH3 domain of the first and second polypeptide chains.

24. The binding protein of claim 22 or 23, wherein the hetero-dimerization motif comprises knobs-into-holes mutations in the CH3 domains of the first and second polypeptide chains.

25. The binding protein of any one of claims 22-24, wherein the second immunoglobulin hinge region comprises an amino acid deletion, insertion or substitution.

26. The binding protein of any one of claims 22-25, wherein the second immunoglobulin hinge region comprises an altered cysteine residue.

27. The binding protein of claim 26, wherein the altered cysteine residue enhances the hetero-dimerization of the first and second polypeptide chains as compared to the hetero-dimerization of first and second polypeptide chains comprising an unmodified second immunoglobulin hinge region.

28. The binding protein of claim 26, wherein the altered cysteine is the N terminal cysteine of the second immunoglobulin hinge region.

29. The binding protein of claim 22, wherein at least one of the first or the second immunoglobulin hinge regions comprises at least 4 continuous amino acids from the amino acid sequence EPKSCDKTHTCPPC.

30. The binding protein of claim 22, wherein the first immunoglobulin hinge region comprises the amino acid sequence EPKSCDKTHT.

31. The binding protein of claim 22, wherein the second immunoglobulin hinge region comprises the amino acid sequence EPKSXDKTHT, wherein X denotes an altered cysteine.

32. The binding protein of claim 22, wherein the second immunoglobulin hinge region comprises the amino acid sequence EPKSXDKTHT, wherein X is any amino acid except cysteine.

33. The binding protein of claim 22, wherein the second immunoglobulin hinge region comprises the amino acid sequence EPKSXDKTHT, wherein X is alanine.

34. The binding protein of claim 22, wherein the DKTHT sequence of the EPKSXDKTHT amino acid sequence within the first immunoglobulin hinge region is replaced with the amino acid sequence VE.

35. The binding protein of claim 33, wherein the EPKSXDKTHT amino acid sequence within the second immunoglobulin hinge region is replaced with the amino acid sequence VE.

36. The binding protein of claim 34, wherein the EPKSXDKTHT amino acid sequence within the second immunoglobulin hinge region is replaced with the amino acid sequence VE.

37. The binding protein of claim 34, wherein the EPKSX sequence of the EPKSXDKTHT amino acid sequence within the second immunoglobulin hinge region is deleted.

38. The binding protein of claim 33, wherein the EPKSX sequence of the EPKSXDKTHT amino acid sequence within the second immunoglobulin hinge region is deleted.

39. The binding protein of any one of claims 22-38, wherein the first and second immunoglobulin hinge regions are IgG1 hinge regions.

40. The binding protein of any one of claims 22-39, wherein the light chain constant domain is a C_{κ} (kappa) constant domain.

41. The binding protein of any one of claims 22-40, wherein the first and second polypeptide chains are covalently linked.

42. The binding protein of any one of claims 22-41, wherein antigens A and B are the same antigen.

43. A binding protein comprising a first and a second polypeptide chain,
wherein the first polypeptide chain comprises VH1-L1-VH2-CH1-X1-CH2-CH3,
wherein
VH1 is a first heavy chain variable domain,
L1 is a linker,
VH2 is a second heavy chain variable domain,
CH1, CH2 and CH3 are heavy chain constant domains 1, 2 and 3, respectively,
X1 comprises a first IgG1 hinge region comprising the amino acid sequence EPKSCDKTHT, and
wherein the second polypeptide chain comprises VL1-L2-VL2-CK-X2-CH2-CH3,
wherein
VL1 is a first light chain variable region,
L2 is a linker,
VL2 is a second light chain variable region,
C_{κ} is a kappa light chain constant domain,
X2 comprises a modified second IgG1 hinge region comprising the amino acid sequence EPKSXDKTHT, wherein X denotes a substitution of a cysteine residue with alanine,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

44. The binding protein of claim 43, wherein the hetero-dimerization motif is located in the CH3 domain of the first and second polypeptide chains.

45. The binding protein of claim 43, wherein the hetero-dimerization motif comprises knobs-into-holes mutations in the CH3 domains of the first and second polypeptide chains.

46. A binding protein comprising a first and a second polypeptide chain,
wherein the first polypeptide chain comprises VH1-L1-VH2-CH1-X1-CH2-CH 3, wherein
VH1 is a first heavy chain variable domain,
L1 is a linker,
VH2 is a second heavy chain variable domain,
CH1, CH2 and CH3 are heavy chain constant domains 1, 2 and 3, respectively,
X1 comprises a first IgG1 hinge region comprising the amino acid sequence EPKSCDKTHT, and
wherein the second polypeptide chain comprises VL1-L2-VL2-Cκ-X2-CH2-CH3,
wherein
VL1 is a first light chain variable region,
L2 is a linker,
VL2 is a second light chain variable region,
Cκ is a kappa light chain constant domain,
X2 comprises a modified second hinge region, wherein EPKSC of the EPKSCDKTHT amino acid sequence is deleted,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

47. The binding protein of claim 46, wherein the hetero-dimerization motif is located in the CH3 domain of the first and second polypeptide chains.

48. The binding protein of claim 46, wherein the hetero-dimerization motif comprises knobs-into-holes mutations in the CH3 domains of the first and second polypeptide chains.

49. A binding protein comprising a first and a second polypeptide chain,
wherein the first polypeptide chain comprises VH1-L1-VH2-CH1-X1-CH2-CH3,
wherein
VH1 is a first heavy chain variable domain,
L1 is a linker,
VH2 is a second heavy chain variable domain,
CH1, CH2 and CH3 are heavy chain constant domains 1,2 and 3, respectively,
X1 comprises a first IgG1 hinge region comprising the amino acid sequence EPKSCDKTHT, and
wherein the second polypeptide chain comprises VL1-L2-VL2-CK-X2-CH2-CH3,
wherein
VL1 is a first light chain variable region,
L2 is a linker,
VL2 is a second light chain variable region,
C_{κ} is a kappa light chain constant domain,
X2 comprises a second modified IgG1 hinge region, wherein the EPKSXDKTHT amino acid sequence is replaced with the amino acid sequence VE,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

50. The binding protein of claim 49, wherein the hetero-dimerization motif is located in the CH3 domain of the first and second polypeptide chains.

51. The binding protein of claim 49, wherein the hetero-dimerization motif comprises knobs-into-holes mutations in the CH3 domains of the first and second polypeptide chains.

52. A binding protein comprising a first and a second polypeptide chain,
wherein the first polypeptide chain comprises VH1-L1-VH2-CH1-X1-CH2-CH3,
wherein
VH1 is a first heavy chain variable domain,
L1 is a linker,
VH2 is a second heavy chain variable domain,
CH1, CH2 and CH3 are heavy chain constant domains 1, 2 and 3, respectively,
X1 comprises a first IgG1 hinge region, wherein the DKTHT sequence of the EPKSXDKTHT amino acid sequence is replaced with the amino acid sequence VE and
wherein the second polypeptide chain comprises VL1-L2-VL2-CK-X2-CH2-CH3,
wherein
VL1 is a first light chain variable region,
L2 is a linker,
VL2 is a second light chain variable region,
C_{κ} is a kappa light chain constant domain,
X2 comprises a modified second IgG1 hinge region comprising the amino acid sequence EPKSXDKTHT, wherein X denotes a substitution of a cysteine residue with alanine,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

53. The binding protein of claim 52, wherein the hetero-dimerization motif is located in the CH3 domain of the first and second polypeptide chains.

54. The binding protein of claim 52, wherein the hetero-dimerization motif comprises knobs-into-holes mutations in the CH3 domains of the first and second polypeptide chains.

55. A binding protein comprising a first and a second polypeptide chain,
wherein the first polypeptide chain comprises VH1-L1-VH2-CH1-X1-CH2-CH3,
wherein
VH1 is a first heavy chain variable domain,
L1 is a linker.
VH2 is a second heavy chain variable domain,
CH1, CH2 and CH3 are heavy chain constant domains 1, 2 and 3, respectively,
X1 comprises a first IgG1 hinge region, wherein the DKTHT sequence of the EPKSXDKTHT amino acid sequence is replaced with the amino acid sequence VE and
wherein the second polypeptide chain comprises VL1-L2-VL2-CK-X2-CH2-CH3,
wherein
VL1 is a first light chain variable region,
L2 is a linker,
VL2 is a second light chain variable region,
C_{κ} is a kappa light chain constant domain,
X2 comprises a modified second IgG1 hinge region, wherein EPKSC of the EPKSCDKTHT amino acid sequence is deleted,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

56. The binding protein of claim 55, wherein the hetero-dimerization motif is located in the CH3 domain of the first and second polypeptide chains.

57. The binding protein of claim 55, wherein the hetero-dimerization motif comprises knobs-into-holes mutations in the CH3 domains of the first and second polypeptide chains.

58. A binding protein comprising a first and a second polypeptide chain,
wherein the first polypeptide chain comprises VH1-L1-VH2-CH1-X1-CH2-CH3,
wherein
VH1 is a first heavy chain variable domain,
L1 is a linker,
VH2 is a second heavy chain variable domain,
CH1, CH2 and CH3 are heavy chain constant domains 1, 2 and 3, respectively,
X1 comprises a first IgG1 hinge region, wherein DKTHT of the EPKSXDKTHT amino acid sequence is replaced with the amino acid sequence VE and
wherein the second polypeptide chain comprises VL1-L2-VL2-CK-X2-CH2-CH3,
wherein
VL1 is a first light chain variable region,
L2 is a linker,
VL2 is a second light chain variable region,
C_{κ} is a kappa light chain constant domain,
X2 comprises a second modified IgG1 hinge region wherein the EPKSXDKTHT amino acid sequence is replaced with the amino acid sequence VE,
CH2 and CH3 are heavy chain constant domains 2 and 3, respectively; and
wherein
the first and second polypeptide chains comprise a hetero-dimerization motif that facilitates the dimerization of the first and second polypeptide chains, and
wherein
VH1 and VL1 form one functional binding site for antigen A, and
VH2 and VL2 form one functional binding site for antigen B.

59. The binding protein of claim 58, wherein the hetero-dimerization motif is located in the CH3 domain of the first and second polypeptide chains.

60. The binding protein of claim 58, wherein the hetero-dimerization motif comprises knobs-into-holes mutations in the CH3 domains of the first and second polypeptide chains.

61. The binding protein of any one of claims 1-60, wherein the binding protein is capable of binding one or more of ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C-Met; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DLL4, DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4;HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL 1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1 MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGE, PGE2, PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRRIB (Spr1); ST6GAL1; STAB1; STAT6; STEAP; STEAP2; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; INFRSF8; TNFRSF9; TNFSF10 (TRAIL); TNFSF11 (TRANCE); TNFSF12 (APO3L); TNFSF13 (April); TNTSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; or ZFPM.

62. A binding protein conjugate comprising the binding protein of any one of claims 1-61, the binding protein conjugate further comprising an immunoadhesion molecule, an imaging agent, a therapeutic agent, or a cytotoxic agent.

63. The binding protein conjugate of claim 62, wherein the imaging agent is a radiolabel, an enzyme, a fluorescent label, a luminescent label, a bioluminescent label, a magnetic label, or biotin.

64. The binding protein conjugate of claim 63, wherein the radiolabel is ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

65. The binding protein conjugate of claim 62, wherein the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent.

66. An isolated nucleic acid encoding the binding protein amino acid sequence according to any one of claims 1-61.

67. A vector comprising the isolated nucleic acid according to claim 66.

68. The vector of claim 67, wherein the vector comprises pcDNA, pTT, pTT3, pEFBOS, pBV, pJV, pcDNA3.1 TOPO, pEF6, pHybE, TOPO, or pBJ.

69. A host cell comprising the vector of claim 67 or 68.

70. The host cell of claim 69, wherein the host cell is a prokaryotic cell, Escherichia coli, a eukaryotic cell, a protist cell, an animal cell, a plant cell, a fungal cell, a yeast cell, an Sf9 cell, a mammalian cell, an avian cell, an insect cell, a CHO cell or a COS cell.

71. A method of producing a binding protein, comprising culturing the host cell of claim 69 or 70 in culture medium under conditions sufficient to produce the binding protein.

72. A pharmaceutical composition comprising the binding protein according to any one of claims 1-61, and a pharmaceutically acceptable carrier.

73. The pharmaceutical composition of claim 72, further comprising at least one additional therapeutic agent.

74. The pharmaceutical composition according to claim 73, wherein the additional therapeutic agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunoglobulin, air immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

75. A method of treating a subject for a disease or a disorder by administering the binding protein of any one of claims 1-61 to the subject.

76. The method of claim 75, wherein the disorder is arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, Yersinia and salmonella associated arthropathy, spondyloarthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, cholestasis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma) abetalipoproteinemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneurysms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrom/thrombolytic thrombocytopenic purpura, hemorrhage, hepatitis A, His bundle arryhthmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignant Lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shy-Drager and Machado-Joseph), myasthenia gravis, mycobacterium avium intracellulare, mycobacterium tuberculosis, myelodyplastic syndrome, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherloselerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, anaphylaxis, anti-phospholipid antibody syndrome, aplastic anemia, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, childhood onset psychiatric disorder, dacryocystitis, dermatomyositis, diabetic retinopathy, disk herniation, disk prolaps, drug induced immune hemolytic anemia, endometriosis, endophthalmitis, episcleritis, erythema multiforme, erythema multiforme major, gestational pemphigoid, Guillain-Barré syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratoconjunctivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myelodysplastic syndrome, myocarditis nerve root disorders, neuropathy, non-A non-B hepatitis, optic neuritis, osteolysis, ovarian cancer, pauciarticular JRA, peripheral artery occlusive disease (PAOD), peripheral vascular disease (PVD), peripheral artery, disease (PAD), phlebitis, polyarteritis nodosa (or periarteritis nodosa), polychondritis, polymyalgia rheumatica, poliosis, polyarticular JRA, polyendocrine deficiency syndrome, polymyositis, post-pump syndrome, primary Parkinsonism, prostate and rectal cancer and hematopoietic malignancies (leukemia and lymphoma), prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor necrosis factor receptor, type 1 allergic reaction, type II diabetes, usual interstitial pneumonia (UIP), vernal conjunctivitis, viral retinitis, Vogt-Koyanagi-Harada syndrome (VKH syndrome), wet macular degeneration, or wound healing.

77. The method of claim 75 or 76, wherein the disorder is an autoimmune disorder, asthma, arthritis, rheumatoid arthritis, osteoarthritis, systemic lupus erythematosus (SLE), multiple sclerosis, sepsis, a neurodegenerative disease, or an oncological disorder.

78. The method of any one of claims 75-77, wherein the binding protein is formulated for parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal administration.

79. A method of detecting the presence, amount, or concentration of at least one target or fragment thereof in a test sample by an immunoassay,
wherein the immunoassay comprises contacting the test sample with at least one binding protein of any one of claims 1-61 and at least one detectable label.

80. The method of claim 79, further comprising:
(i) contacting the test sample with the at least one binding protein, wherein the binding protein binds to an epitope on the target or fragment thereof so as to form a first complex;
(ii) contacting the complex with the at least one detectable label, wherein the detectable label binds to the binding protein or an epitope on the target or fragment thereof that is not bound by the binding protein to form a second complex; and
(iii) detecting the presence, amount, or concentration of the target or fragment thereof in the test sample based on the signal generated by the detectable label in the second complex, wherein the presence, amount, or concentration of the target or fragment thereof is directly correlated with the signal generated by the detectable label.

81. The method of claim 79, further comprising:
(i) contacting the test sample with the at least one binding protein, wherein the binding protein binds to an epitope on the target or fragment thereof so as to form a first complex;
(ii) contacting the complex with the at least one detectable label, wherein the detectable label competes with the target or fragment thereof for binding to the binding protein so as to form a second complex; and
(iii) detecting the presence, amount, or concentration of the target or fragment thereof in the test sample based on the signal generated by the detectable label in the second complex, wherein the presence, amount, or concentration of the target or fragment thereof is indirectly correlated with the signal generated by the detectable label.

82. A kit for assaying a test sample for the presence, amount, or concentration of a target or fragment thereof in the sample, the kit comprising (a) instructions for assaying the test sample for the target or fragment thereof and (b) at least one binding protein comprising the binding protein of any one of claims 1-61.

83. Use of the binding protein of any one of claims 1-61 in the manufacture of a medicament for treating a disease or disorder.


SEQUENCE LISTING (72 sequences)

SEQ ID NO: 1 - protein, 118 aa, Artificial Sequence
  source: /note=“Description of Artificial Sequence Synthetic polypeptide”
EVQLVESGGGLVQPGGSLRLSCAASGFTFSNYGVTWVRQAPGKGLEWVSMIWADGSTHYASSVKGRFTISRDNSKNTLYLQMNSLRAEDTAVYYCAREWQHGPVAYWGQGTLVTYVSS

SEQ ID NO: 2 - protein, 121 aa, Artificial Sequence
  source: /note=“Description of Artificial Sequence Synthetic polypeptide”
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTIVTVSS

SEQ ID NO: 3 - protein, 123 aa, Artificial Sequence
  source: /note=“Description of Artificial Sequence Synthetic polypeptide”
EVQLVESGGGLVQPGRSLRLSCAASGFTFDHYAIVIKWVRQAPGKGLEWVSAITWNSGHIDYADSVEGRFTISRDNAKNSLYLQMNSLRAEDTAVYYCAKVSYLSTASSLDYWGQGTIVTVSS

SEQ ID NO: 4 - protein, 10 aa, Artificial Sequence
  source: /note=“Description of Artificial Sequence Synthetic peptide”
GGGGSGGGGS

SEQ ID NO: 5 - protein, 126 aa, Artificial Sequence
  source: /note=“Description of Artificial Sequence Synthetic polypeptide”
EVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWGGYYSTHDFDSWGQGTTVTVSS

SEQ ID NO: 6 - protein, 108 aa, Artificial Sequence
  source: /note=“Description of Artificial Sequence Synthetic polypeptide”
DIQMTQSPSSLSASVGDRVTITCRASQLVSSAVAWVQQKPGKAPKLLIYWASARHTGVPSRFSGSGSGTDFTLTISSLQPEDFATYYCQQHYKTPFTFGQGTKLEIKR

SEQ ID NO: 7 - protein, 108 aa, Artificial Sequence
  source: /note=“Description of Artificial Sequence Synthetic polypeptide”
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 8 - protein, 107 aa, Artificial Sequence
  source: /note=“Description of Artificial Sequence Synthetic polypeptide”
DIQMTQSPSSLSASVGDRVTITCRASHSIRNYLSWYQQKPGKAPKLLIYAASTLQSGVPSRFSGSGSGTDFTTISSLQPEDVATYYCQRYNRAPYTFGQGTKVEIKR

SEQ ID NO: 9 - protein, 9 aa, Artificial Sequence
  source: /note=“Description of Artificial Sequence Synthetic peptide”
GGSGGGGSG

SEQ ID NO: 10 - protein, 108 aa, Artificial Sequence
  source: /note=“Description of Artificial Sequence Synthetic polypeptide”
EIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 11 - protein, 14 aa, Artificial Sequence
  source: /note=“Description of Artificial Sequence Synthetic peptide”
EPKSCDKTHTCPPC

SEQ ID NO: 12 - protein, 6 aa, Artificial Sequence
  source: /note=“Description of Artificial Sequence Synthetic peptide”
VECPPC

SEQ ID NO: 13 - protein, 14 aa, Artificial Sequence
  source: /note=“Description of Artificial Sequence Synthetic peptide”
EPKSADKTHTCPPC

SEQ ID NO: 14 - protein, 9 aa, Artificial Sequence
  source: /note=“Description of Artificial Sequence Synthetic peptide”
DKTHTCPPC

SEQ ID NO: 15 - protein, 6 aa, Artificial Sequence
  source: /note=“Description of Artificial Sequence Synthetic peptide”
VECPPC

SEQ ID NO: 16 - protein, 19 aa, Artificial Sequence
  source: /note=“Description of Artificial Sequence Synthetic peptide”
VDKKVEPKSCDKTHTCPPC

SEQ ID NO: 17 - protein, 19 aa, Artificial Sequence
  source: /note=“Description of Artificial Sequence Synthetic peptide”
NRGECEPKSADKTHTCPPC

SEQ ID NO: 18 - protein, 19 aa, Artificial Sequence
  source: /note=“Description of Artificial Sequence Synthetic peptide”
VDKKVEPKSCDKTHTCPPC

SEQ ID NO: 19 - protein, 19 aa, Artificial Sequence
  source: /note=“Description of Artificial Sequence Synthetic peptide”
NRGECDKTHTCPPCPAPEL

SEQ ID NO: 20 - protein, 19 aa, Artificial Sequence
  source: /note=“Description of Artificial Sequence Synthetic peptide”
VDKKVEPKSCDKTHTCPPC

SEQ ID NO: 21 - protein, 19 aa, Artificial Sequence
  source: /note=“Description of Artificial Sequence Synthetic peptide”
NRGECVECPPCPAPELLGG

SEQ ID NO: 22 - protein, 19 aa, Artificial Sequence
  source: /note=“Description of Artificial Sequence Synthetic peptide”
VDKKVEPKSCVECPPCPAP

SEQ ID NO: 23 - protein, 19 aa, Artificial Sequence
  source: /note=“Description of Artificial Sequence Synthetic peptide”
NRGECEPKSADKTHTCPPC

SEQ ID NO: 24 - protein, 19 aa, Artificial Sequence
  source: /note=“Description of Artificial Sequence Synthetic peptide”
VDKKVEPKSCVECPPCPAP

SEQ ID NO: 25 - protein, 19 aa, Artificial Sequence
  source: /note=“Description of Artificial Sequence Synthetic peptide”
NRGECDKTHTCPPCPAPEL

SEQ ID NO: 26 - protein, 19 aa, Artificial Sequence
  source: /note=“Description of Artificial Sequence Synthetic peptide”
VDKKVEPKSCVECPPCPAP

SEQ ID NO: 27 - protein, 19 aa, Artificial Sequence
  source: /note=“Description of Artificial Sequence Synthetic peptide”
NRGECVECPPCPAPELLGG

SEQ ID NO: 28 - protein, 26 aa, Artificial Sequence
  source: /note=“Description of Artificial Sequence Synthetic peptide”
ASTKGPSVFPLAPASTKGPSVFPLAP

SEQ ID NO: 29 - protein, 10 aa, Artificial Sequence
  source: /note=“Description of Artificial Sequence Synthetic peptide”
GGGGSGGGGS

SEQ ID NO: 30 - protein, 9 aa, Artificial Sequence
  source: /note=“Description of Artificial Sequence Synthetic peptide”
GGSGGGGSG

SEQ ID NO: 31 - protein, 5 aa, Artificial Sequence
  source: /note=“Description of Artificial Sequence Synthetic peptide”
GGGGS

SEQ ID NO: 32 - protein, 10 aa, Artificial Sequence
  source: /note=“Description of Artificial Sequence Synthetic peptide”
EPKSCDKTHT

SEQ ID NO: 33 - protein, 10 aa, Artificial Sequence
  source: /note=“Description of Artificial Sequence Synthetic peptide”
EPKSCDKTHT

SEQ ID NO: 34 - protein, 10 aa, Artificial Sequence
  source: /note=“Description of Artificial Sequence Synthetic peptide”
EPKSXDKTHT

SEQ ID NO: 35 - protein, 10 aa, Artificial Sequence
  source: /note=“Description of Artificial Sequence Synthetic peptide”
EPKSADKTHT

SEQ ID NO: 36 - protein, 5 aa, Artificial Sequence
  source: /note=“Description of Artificial Sequence Synthetic peptide”
DKTHT

SEQ ID NO: 37 - protein, 10 aa, Artificial Sequence
  source: /note=“Description of Artificial Sequence Synthetic peptide”
EPKSXDKTHT

SEQ ID NO: 38 - protein, 5 aa, Artificial Sequence
  source: /note=“Description of Artificial Sequence Synthetic peptide”
EPKSX

SEQ ID NO: 39 - protein, 5 aa, Artificial Sequence
  source: /note=“Description of Artificial Sequence Synthetic peptide”
EPKSC

SEQ ID NO: 40 - protein, 16 aa, Artificial Sequence
  source: /note=“Description of Artificial Sequence Synthetic peptide”
AKTTPKLEEGEFSEAR

SEQ ID NO: 41 - protein, 17 aa, Artificial Sequence
  source: /note=“Description of Artificial Sequence Synthetic peptide”
AKTTPKLEEGEFSEARV

SEQ ID NO: 42 - protein, 9 aa, Artificial Sequence
  source: /note=“Description of Artificial Sequence Synthetic peptide”
AKTTPKLGG

SEQ ID NO: 43 - protein, 10 aa, Artificial Sequence
  source: /note=“Description of Artificial Sequence Synthetic peptide”
SAKTTPKLGG

SEQ ID NO: 44 - protein, 6 aa, Artificial Sequence
  source: /note=“Description of Artificial Sequence Synthetic peptide”
SAKTTP

SEQ ID NO: 45 - protein, 6 aa, Artificial Sequence
  source: /note=“Description of Artificial Sequence Synthetic peptide”
RADAAP

SEQ ID NO: 46 - protein, 9 aa, Artificial Sequence
  source: /note=“Description of Artificial Sequence Synthetic peptide”
RADAAPTVS

SEQ ID NO: 47 - protein, 12 aa, Artificial Sequence
  source: /note=“Description of Artificial Sequence Synthetic peptide”
RADAAAAGGPGS

SEQ ID NO: 48 - protein, 27 aa, Artificial Sequence
  source: /note=“Description of Artificial Sequence Synthetic peptide”
RADAAAAGGGGSGGGGSGGGGSGGGGS

SEQ ID NO: 49 - protein, 18 aa, Artificial Sequence
  source: /note=“Description of Artificial Sequence Synthetic peptide”
SAKTTPKLEEGEFSEARV

SEQ ID NO: 50 - protein, 5 aa, Artificial Sequence
  source: /note=“Description of Artificial Sequence Synthetic peptide”
ADAAP

SEQ ID NO: 51 - protein, 12 aa, Artificial Sequence
  source: /note=“Description of Artificial Sequence Synthetic peptide”
ADAAPTVSIFPP

SEQ ID NO: 52 - protein, 5 aa, Artificial Sequence
  source: /note=“Description of Artificial Sequence Synthetic peptide”
TVAAP

SEQ ID NO: 53 - protein, 12 aa, Artificial Sequence
  source: /note=“Description of Artificial Sequence Synthetic peptide”
TVAAPSVFIFPP

SEQ ID NO: 54 - protein, 6 aa, Artificial Sequence
  source: /note=“Description of Artificial Sequence Synthetic peptide”
QPKAAP

SEQ ID NO: 55 - protein, 13 aa, Artificial Sequence
  source: /note=“Description of Artificial Sequence Synthetic peptide”
QPKAAPSVTLFPP

SEQ ID NO: 56 - protein, 6 aa, Artificial Sequence
  source: /note=“Description of Artificial Sequence Synthetic peptide”
AKTTPP

SEQ ID NO: 57 - protein, 13 aa, Artificial Sequence
  source: /note=“Description of Artificial Sequence Synthetic peptide”
AKTTPPSVTPLAP

SEQ ID NO: 58 - protein, 6 aa, Artificial Sequence
  source: /note=“Description of Artificial Sequence Synthetic peptide”
AKTTAP

SEQ ID NO: 59 - protein, 13 aa, Artificial Sequence
  source: /note=“Description of Artificial Sequence Synthetic peptide”
AKTTAPSVYPLAP

SEQ ID NO: 60 - protein, 6 aa, Artificial Sequence
  source: /note=“Description of Artificial Sequence Synthetic peptide”
ASTKGP

SEQ ID NO: 61 - protein, 13 aa, Artificial Sequence
  source: /note=“Description of Artificial Sequence Synthetic peptide”
ASTKGPSVFPLAP

SEQ ID NO: 62 - protein, 15 aa, Artificial Sequence
  source: /note=“Description of Artificial Sequence Synthetic peptide”
GGGGSGGGGSGGGGS

SEQ ID NO: 63 - protein, 15 aa, Artificial Sequence
  source: /note=“Description of Artificial Sequence Synthetic peptide”
GENKVEYAPALMALS

SEQ ID NO: 64 - protein, 15 aa, Artificial Sequence
  source: /note=“Description of Artificial Sequence Synthetic peptide”
GPAKELTPLKEAKVS

SEQ ID NO: 65 - protein, 15 aa, Artificial Sequence
  source: /note=“Description of Artificial Sequence Synthetic peptide”
GHEAAAVMQVQYPAS

SEQ ID NO: 66 - protein, 24 aa, Artificial Sequence
  source: /note=“Description of Artificial Sequence Synthetic peptide”
TVAAPSVFIFPPTVAAPSVFIFPP

SEQ ID NO: 67 - protein, 5 aa, Artificial Sequence
  source: /note=“Description of Artificial Sequence Synthetic peptide”
EPKSA

SEQ ID NO: 68 - protein, 5 aa, Artificial Sequence
  source: /note=“Description of Artificial Sequence Synthetic peptide”
NRGEC

SEQ ID NO: 69 - protein, 10 aa, Artificial Sequence
  source: /note=“Description of Artificial Sequence Synthetic peptide”
VDKKVEPKSC

SEQ ID NO: 70 - protein, 10 aa, Artificial Sequence
  source: /note=“Description of Artificial Sequence Synthetic peptide”
NRGECEPKSC

SEQ ID NO: 71 - protein, 11 aa, Artificial Sequence
  source: /note=“Description of Artificial Sequence Synthetic peptide”
EPKSCVECPPC

SEQ ID NO: 72 - protein, 7 aa, Artificial Sequence
  source: /note=“Description of Artificial Sequence Synthetic peptide”
CVECPPC